(19) United States
(12) Patent Application Publication
(10) Pub. No.: US 2023/0181272 A1
(43) Pub. Date: Jun. 15, 2023

(54) Robotic Arm With Hybrid Actuation Assemblies And Related Devices, Systems, And Methods

(71) Applicant: Virtual Incision Corporation, Lincoln, NE (US)

(72) Inventors: Shane Farritor, Lincoln, NE (US); Parker Durham, Lincoln, NE (US); Nathan Wood, Lincoln, NE (US); David G. Matsuura, Solana Beach, CA (US); Philip J. Simpson, Escondido, CA (US); Belinko K. Matsuura, Encinitas, CA (US); Dalton W. Wilmers, San Diego, CA (US)

(21) Appl. No.: 18/064,784

(22) Filed: Dec. 12, 2022

Related U.S. Application Data
(60) Provisional application No. 63/288,486, filed on Dec. 10, 2021.

(51) Int. Cl. A61B 34/32; B25J 9/10; A61B 34/00; A61B 17/29

(52) U.S. Cl. CPC A61B 34/32; B25J 9/104; B25J 9/102; A61B 34/71; A61B 17/29; A61B 2034/301

(57) ABSTRACT

Robotic arms, and devices with such arms, having any combination of gear-driven actuator assemblies and cable-driven actuator assemblies, with some arm or device embodiments having solely gear-driven assemblies, some having solely cable-driven assemblies, and others having a combination of at least one of each. Further embodiments relate to arms or devices having one or more actuation assemblies with an actuator is disposed remotely (in a different component of the device—or even external to the device) in relation to the actuable component to which it is coupled.

### CROSS-REFERENCE TO RELATED APPLICATION(S)

This application claims the benefit under 35 U.S.C. § 119(e) to U.S. Provisional Application 63/288,486, filed Dec. 10, 2021 and entitled “Robotic Arm with Hybrid Actuation Assemblies and Related Devices, Systems, and Methods,” which is hereby incorporated herein by reference in its entirety.

### FIELD

The embodiments disclosed herein relate to various medical devices and related components that can make up a surgical system, including robotic and/or in vivo medical devices and related components. Certain embodiments include various robotic medical devices, including robotic devices that are disposed within a body cavity and positioned using a body or support component disposed through an orifice or opening in the body cavity. Other embodiments relate to various systems that have a robotic surgical device and a controller.

### BACKGROUND

Invasive surgical procedures are essential for addressing various medical conditions. When possible, minimally invasive procedures such as laparoscopy are preferred.

However, known minimally invasive technologies such as laparoscopy are limited in scope and complexity due in part to 1) mobility restrictions resulting from using rigid tools inserted through access ports, and 2) limited visual feedback. Known robotic systems such as the da Vinci® Surgical System (available from Intuitive Surgical, Inc., located in Sunnyvale, Calif.) are also restricted by the access ports, as well as having the additional disadvantages of being very large, very expensive, unavailable in most hospitals, and having limited sensory and mobility capabilities.

There is a need in the art for improved surgical methods, systems, and devices.

### BRIEF SUMMARY

Discussed herein are various robotic arms that can be actuated by any combination of gear-driven actuator assemblies and cable-driven actuator assemblies, with some embodiments having solely gear-driven assemblies, some having solely cable-driven assemblies, and others having a combination of at least one of each. Further implementations herein relate to various such actuation assemblies in which the actuator is disposed remotely (in a different component of the device—or event external to the device—in relation to the actuable component to which it is coupled), and to devices having at least one such remotely positioned actuation assembly. Other embodiments relate to robotic devices and/or robotic surgical systems having any of the various actuation assembly implementations described herein.

In Example 1, a robotic device comprises an elongate body and a robotic arm operably coupled to the elongate body. The robotic arm comprises an upper arm segment, a forearm segment operably coupled to the upper arm segment, and at least two actuable components associated with the robotic arm. The device further comprises a first cable-driven actuation assembly comprising a first actuator disposed within one of the elongate body, the upper arm segment, and the forearm segment or external to the device, and a motive force transfer cable operably coupled to the first actuator and a first of the at least two actuable components. In addition, the device comprises a first gear-driven actuation assembly comprising a second actuator disposed within one of the elongate body, the upper arm segment, and the forearm segment or external to the device, and at least one gear operably coupled to the second actuator and a second of the at least two actuable components.

Example 2 relates to the robotic device according to Example 1, wherein the motive force transfer cable is a rotary force transfer cable.

Example 3 relates to the robotic device according to Example 1, wherein the motive force transfer cable is a lateral force transfer cable.

Example 4 relates to the robotic device according to Example 3, wherein the lateral force transfer cable comprises a single lateral push/pull cable or two lateral pull cables.

Example 5 relates to the robotic device according to Example 1, wherein the first of the at least two actuable components comprises actuable end effector grasper arms, and wherein the second of the at least two actuable components comprises a rotatable end effector grasper body.

Example 6 relates to the robotic device according to Example 1, wherein the first and second actuators are disposed within the forearm segment.

Example 7 relates to the robotic device according to Example 1, wherein the first actuator is disposed within the upper arm segment and the second actuator is disposed within the forearm segment.

Example 8 relates to the robotic device according to Example 1, wherein the first actuator is disposed within the elongate body and the second actuator is disposed within the forearm segment.

Example 9 relates to the robotic device according to Example 1, wherein the first actuator is disposed external to the device and the second actuator is disposed within the forearm segment.

In Example 10, a robotic device comprises an elongate body and a robotic arm operably coupled to the elongate body. The robotic arm comprises an upper arm segment, a forearm segment operably coupled to the upper arm segment, and at least two actuable components associated with the robotic arm. The device also comprises a first cable-driven actuation assembly comprising a first actuator disposed within one of the elongate body, the upper arm segment, and the forearm segment or external to the device, and a first motive force transfer cable operably coupled to the first actuator and a first of the at least two actuable components. Further, the device also comprises a second cable-driven actuation assembly comprising a second actuator disposed within one of the elongate body, the upper arm segment, and the forearm segment or external to the device, and a second motive force transfer cable operably coupled to the second actuator and a second of the at least two actuable components.

Example 11 relates to the robotic device according to Example 10, further comprising a first gear-driven actuation assembly comprising a third actuator disposed within one of the elongate body, the upper arm segment, and the forearm segment or external to the device, and at least one gear operably coupled to the third actuator and a third of the at least two actuable components.

Example 12 relates to the robotic device according to Example 10, wherein at least one of the first and second motive force transfer cables is a rotary force transfer cable.

Example 13 relates to the robotic device according to Example 10, wherein at least one of the first and second motive force transfer cables is a lateral force transfer cable.

Example 14 relates to the robotic device according to Example 10, wherein the first of the at least two actuable components comprises actuable end effector grasper arms, and wherein the second of the at least two actuable components comprises a rotatable end effector grasper body.

Example 15 relates to the robotic device according to Example 10, wherein the first and second actuators are disposed within the forearm segment.

Example 16 relates to the robotic device according to Example 10, wherein the first and second actuators are disposed external to the device.

In Example 17, a robotic device comprises an elongate body, an actuation unit coupled to the elongate body, the actuation unit comprising at least two actuators, and a robotic arm operably coupled to the elongate body. The robotic arm comprises an upper arm segment, a forearm segment operably coupled to the upper arm segment, and at least two actuable components associated with the robotic arm. The device also comprises a first cable-driven actuation assembly comprising a first actuator disposed within the actuation unit, and a first rotary force transfer cable operably coupled to the first actuator and a first of the at least two actuable components. Further, the device also comprises a second cable-driven actuation assembly comprising a second actuator disposed within the actuation unit, and a second rotary force transfer cable operably coupled to the second actuator and a second of the at least two actuable components.

Example 18 relates to the robotic device according to Example 17, wherein the first and second rotary force transfer cables are disposed through the elongate body.

Example 19 relates to the robotic device according to Example 18, further comprising a cable positioning block movably disposed within the elongate body, wherein the cable positioning block is operably coupled to the first rotary force transfer cable, and wherein the second rotary force transfer cable is attached to the cable positioning block.

Example 20 relates to the robotic device according to Example 17, wherein the first rotary force transfer cable is disposed through an opening in the cable positioning block such that the first rotary force transfer cable is rotatably coupled to the cable positioning block such that rotation of the first rotary force transfer cable results in axial movement of the cable positioning block with in the elongate body.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a perspective view of a robotic surgical system in an operating room, according to one embodiment.

FIG. 2 is perspective view of a robotic device, according to one embodiment.

FIG. 3A is another perspective view of the robotic device of FIG. 2, according to one embodiment.

FIG. 3B is a perspective view of a camera that is insertable into the robotic device of FIG. 3A, according to one embodiment.

FIG. 4A is an expanded perspective view of the distal end and robotic arms of the robotic device of FIG. 2, according to one embodiment.

FIG. 4B is the expanded perspective view of the distal end and robotic arms of the robotic device of FIG. 2 with the various axes of rotation shown, according to another embodiment.

FIG. 5A is another expanded perspective view of the distal end and robotic arms of the robotic device of FIG. 4A, according to one embodiment.

FIG. 5B is an expanded side view of the distal end and robotic arms of the robotic device of FIG. 4A, according to one embodiment.

FIG. 6 is a schematic perspective view of a robotic device and a camera component that is not positioned in the device and is instead being operated via a separate port in a patient, according to one embodiment.

FIG. 7 is a perspective view of a surgical robotic device showing the internal components, according to one implementation.

FIG. 8 is a front view showing the internal components of the body and shoulders, according to one embodiment.

FIG. 9 is a perspective view showing the internal components of the body, according to one embodiment

FIG. 10 is a perspective view showing the internal components of the shoulders, according to one embodiment.

FIG. 11 is a side view showing the internal components of the shoulders, according to one embodiment.

FIG. 12 is a reverse perspective view showing the internal components of the body and shoulders, according to one embodiment.

FIG. 13 is a perspective view showing the internal components of the upper arm, according to one embodiment.

FIG. 14 is a perspective view showing further internal components of the upper arm, according to one embodiment.

FIG. 15 is a front view showing further internal components of the upper arm, according to one embodiment.

FIG. 16 is a perspective view showing further internal components of the upper arm, according to one embodiment.

FIG. 17 is a perspective view showing internal components of the lower arm, according to one embodiment.

FIG. 18 is a perspective view showing further internal components of the upper arm, according to one embodiment.

FIG. 19 is a perspective view showing further internal components of the upper arm, according to one embodiment.

FIG. 20 is a perspective view showing yet further internal components of the upper arm, according to one embodiment.

FIG. 21A is an expanded schematic view of a robotic right arm of a robotic device with two actuation assemblies therein, according to one embodiment.

FIG. 21B is a schematic view of the robotic device of which the right arm is shown in FIG. 21A, according to one embodiment.

FIG. 22A is a perspective view of some of the internal components of a forearm in which a distal portion is detached from a proximal portion of the forearm, according to one embodiment.

FIG. 22B is another perspective view of the forearm of FIG. 22A in which the two portions are attached, according to one embodiment.

FIG. 23 is a perspective view of the distal portion of the forearm, according to one embodiment.

FIG. 24A is a perspective view of the distal portion of the forearm as shown in FIG. 23 in which the graspers are depicted in one position around the open/close axis, according to one embodiment.

FIG. 24B is a perspective view of the distal portion of the forearm as shown in FIG. 23 in which the graspers are depicted in another position around the open/close axis, according to one embodiment.

FIG. 24C is a perspective view of the distal portion of the forearm as shown in FIG. 23 in which the graspers are depicted in yet another position around the open/close axis, according to one embodiment.

FIG. 25A is a perspective view of the distal portion of the forearm as shown in FIG. 23 in which the graspers are depicted in one position around the wrist yaw axis, according to one embodiment.

FIG. 25B is a perspective view of the distal portion of the forearm as shown in FIG. 23 in which the graspers are depicted in another position around the wrist yaw axis, according to one embodiment.

FIG. 25C is a perspective view of the distal portion of the forearm as shown in FIG. 23 in which the graspers are depicted in yet another position around the wrist yaw axis, according to one embodiment.

FIG. 26A is a perspective view of the distal portion of the forearm depicting the inner mechanisms of the actuation assembly for actuating rotation of one of the paddles around axis A1, according to one embodiment.

FIG. 26B is a different perspective view of the distal portion of the forearm of FIG. 26A depicting the inner mechanisms of the same actuation assembly, according to one embodiment.

FIG. 27A is a perspective view of the distal portion of the forearm depicting the inner mechanisms of the actuation assembly for actuating rotation of the other of the paddles around axis A1, according to one embodiment.

FIG. 27B is a different perspective view of the distal portion of the forearm of FIG. 27A depicting the inner mechanisms of the same actuation assembly, according to one embodiment.

FIG. 28A is a perspective view of the distal portion of the forearm depicting the inner mechanisms of the actuation assembly for actuating rotation of the grasper assembly around axis B1, according to one embodiment.

FIG. 28B is a different perspective view of the distal portion of the forearm of FIG. 27A depicting the inner mechanisms of the same actuation assembly, according to one embodiment.

FIG. 29A is a schematic view of a robotic device with two cable-driven actuation assemblies disposed within the right forearm, according to one embodiment.

FIG. 29B is a schematic view of a robotic device with a cable-driven actuation assembly having an actuator disposed within the upper arm, according to one embodiment.

FIG. 29C is a schematic view of a robotic device with a cable-driven actuation assembly having an actuator disposed within the elongate body, according to one embodiment.

FIG. 29D is a schematic view of a robotic device with a cable-driven actuation assembly having an actuator disposed external to the device, according to one embodiment.

FIG. 30A is an expanded schematic view of a robotic right arm of a robotic device with one gear-driven actuation assembly and one cable-driven actuation assembly therein, according to one embodiment.

FIG. 30B is a schematic view of the robotic device of which the right arm is shown in FIG. 21A, according to one embodiment.

FIG. 31A is a schematic view of a robotic device with a gear-driven actuation assembly disposed within the right forearm, according to one embodiment.

FIG. 31B is a schematic view of a robotic device with a cable-driven actuation assembly disposed within the right forearm, according to one embodiment.

FIG. 31C is a schematic view of a robotic device with a cable-driven actuation assembly having an actuator disposed within the upper arm, according to one embodiment.

FIG. 31D is a schematic view of a robotic device with a cable-driven actuation assembly having an actuator disposed within the elongate body, according to one embodiment.

FIG. 32 is front view of a robotic device (such as the device depicted in FIG. 2) being held by a medical professional, according to one embodiment.

FIG. 33A is a top schematic view of a robotic device positioned such that the arms can access the rectum in a patient, according to one embodiment.

FIG. 33B is a top schematic view of the robotic device of FIG. 33A positioned such that the arms can access the colon in the patient, according to one embodiment.

FIG. 33C is a top schematic view of the robotic device of FIG. 33A positioned such that the arms can access the transverse colon in the patient, according to one embodiment.

FIG. 34A is a schematic view of a prior art system with a drive unit attached to a floor cart.

FIG. 34B is a schematic view of a prior art system with a drive unit on the floor.

FIG. 35A is a perspective view of a robotic device with an external actuation unit, according to one embodiment.

FIG. 35B is a front view of the robotic device of FIG. 35A, according to one embodiment.

FIG. 35C is another front view of the robotic device of FIG. 35A in which the arms are in their extended (straight) position, according to one embodiment.

FIG. 35D is a side view of the robotic device of FIG. 35A in which the arms are in their deployed or bent position, according to one embodiment.

FIG. 35E is a side view of the robotic device of FIG. 35A in which the arms are in their extended (straight) position, according to one embodiment.

FIG. 36A is an expanded perspective view of a proximal portion of the device of FIG. 35A with the actuation unit attached thereto, according to one embodiment.

FIG. 36B is another expanded perspective view of the proximal portion and actuation unit of FIG. 36A, according to one embodiment.

FIG. 36C is another expanded perspective, cutaway view of the proximal portion and actuation unit of FIG. 36A, according to one embodiment.

FIG. 36D is another expanded perspective, cutaway view of the proximal portion and actuation unit of FIG. 36A, according to one embodiment.

FIG. 37A is an expanded perspective view of an actuation unit, according to one embodiment.

FIG. 37B is another expanded perspective view of the actuation unit of FIG. 37A, according to one embodiment.

FIG. 37C is an expanded top view of the actuation unit of FIG. 37A, according to one embodiment.

FIG. 38A is a perspective view of the internal components of an elongate body, according to one embodiment.

FIG. 38B is another perspective view of the internal component of the elongate body of FIG. 38A in which the cable actuation block has moved, according to one embodiment.

FIG. 38C is an expanded perspective view of the internal components of the elongate body of FIG. 38A, according to one embodiment.

FIG. 38D is another expanded perspective view of the internal component of the elongate body of FIG. 38A in which the cable actuation block has moved, according to one embodiment.

FIG. 39 is an expanded perspective view of a distal portion and robotic arms of the robotic device of FIG. 35A, according to one embodiment.

FIG. 40A is an expanded perspective view of a distal portion of the robotic device of FIG. 35A with a right arm in its extended or straight configuration, according to one embodiment.

FIG. 40B is an expanded perspective view of the right arm of FIG. 40A in which the forearm is disposed at an angle in relation to the upper arm, according to one embodiment.

FIG. 40C is an expanded perspective view of the right arm of FIG. 40A in which the upper arm is disposed at an angle in relation to the elongate body and the forearm is disposed at an angle in relation to the upper arm, according to one embodiment.

FIG. 40D is an expanded perspective view of the right arm of FIG. 40A in which the upper arm is disposed at an angle in relation to the elongate body, according to one embodiment.

FIG. 41A is a perspective view depicting an actuation assembly disposed within the elongate body and attached to the shoulder housing, according to one embodiment.

FIG. 41B is another perspective view of the actuation assembly of FIG. 41A, according to one embodiment.

FIG. 410 is another perspective view of the actuation assembly of FIG. 41A, according to one embodiment.

FIG. 42A is a perspective view depicting another actuation assembly disposed within the elongate body and the shoulder housing and attached to the upper arm, according to one embodiment.

FIG. 42B is another perspective view of the actuation assembly of FIG. 42A, according to one embodiment.

FIG. 43A is an expanded side view of the right arm of FIG. 42A in which the upper arm is substantially straight in relation to the elongate body, according to one embodiment.

FIG. 43B is an expanded side view of the right arm of FIG. 42A in which the upper arm is disposed at an angle in relation to the elongate body, according to one embodiment.

FIG. 43C is an expanded side view of the right arm of FIG. 42A in which the upper arm is disposed at a different angle in relation to the elongate body, according to one embodiment.

FIG. 44A is a perspective view depicting another actuation assembly disposed within the upper arm, according to one embodiment.

FIG. 44B is another perspective view of the actuation assembly of FIG. 44A, according to one embodiment.

FIG. 44C is another perspective view of the actuation assembly of FIG. 44A, according to one embodiment.

FIG. 44D is another perspective view of the actuation assembly of FIG. 44A, according to one embodiment.

FIG. 44E is another perspective view of the actuation assembly of FIG. 44A, according to one embodiment.

FIG. 45A is a perspective view depicting another actuation assembly disposed within the upper arm and attached to the forearm, according to one embodiment.

FIG. 45B is another perspective cutaway view of the actuation assembly of FIG. 45A, according to one embodiment.

FIG. 45C is another perspective cutaway view of the actuation assembly of FIG. 45A, according to one embodiment.

FIG. 45D is another perspective cutaway view of the actuation assembly of FIG. 45A, according to one embodiment.

FIG. 45E is another perspective cutaway view of the actuation assembly of FIG. 45A, according to one embodiment.

FIG. 46A is a perspective view depicting another actuation assembly disposed within the forearm and attached to the end effector, according to one embodiment.

FIG. 46B is another perspective view of the actuation assembly of FIG. 46A, according to one embodiment.

FIG. 46C is another perspective view of the actuation assembly of FIG. 46A, according to one embodiment.

FIG. 47 is a perspective view of a grasper end effector, according to one embodiment.

FIG. 48A is a perspective view depicting another actuation assembly disposed within the forearm and attached to the end effector, according to one embodiment.

FIG. 48B is another perspective view of the actuation assembly of FIG. 48A, according to one embodiment.

FIG. 48C is another perspective view of the actuation assembly of FIG. 48A, according to one embodiment.

FIG. 49A is a side view depicting the distal portion of the actuation assembly of FIG. 48A coupled to the graspers, according to one embodiment.

FIG. 49B is another side view depicting the distal portion of the actuation assembly of FIG. 48A coupled to the graspers, according to one embodiment.

FIG. 49C is another side view depicting the distal portion of the actuation assembly of FIG. 48A coupled to the graspers, according to one embodiment.

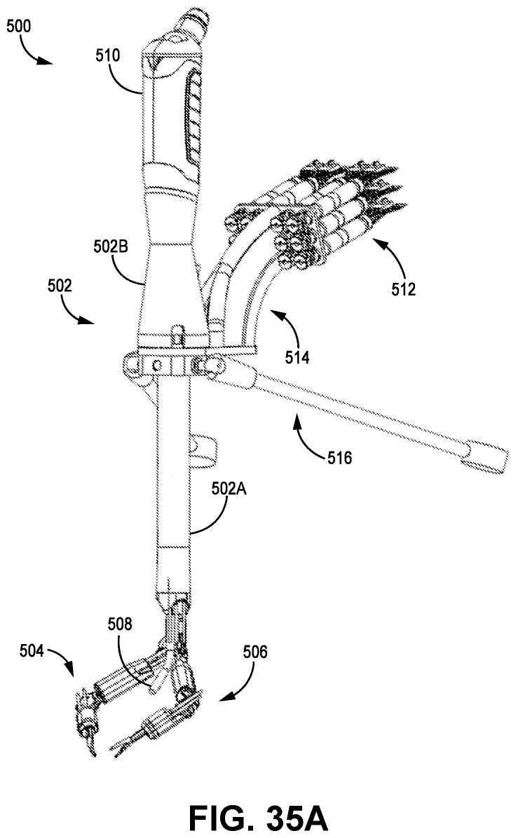

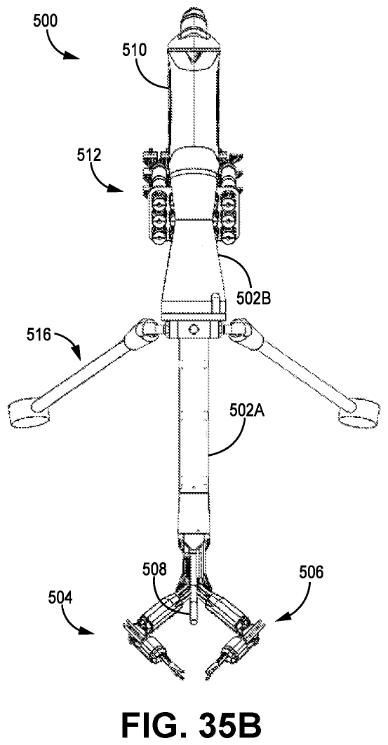

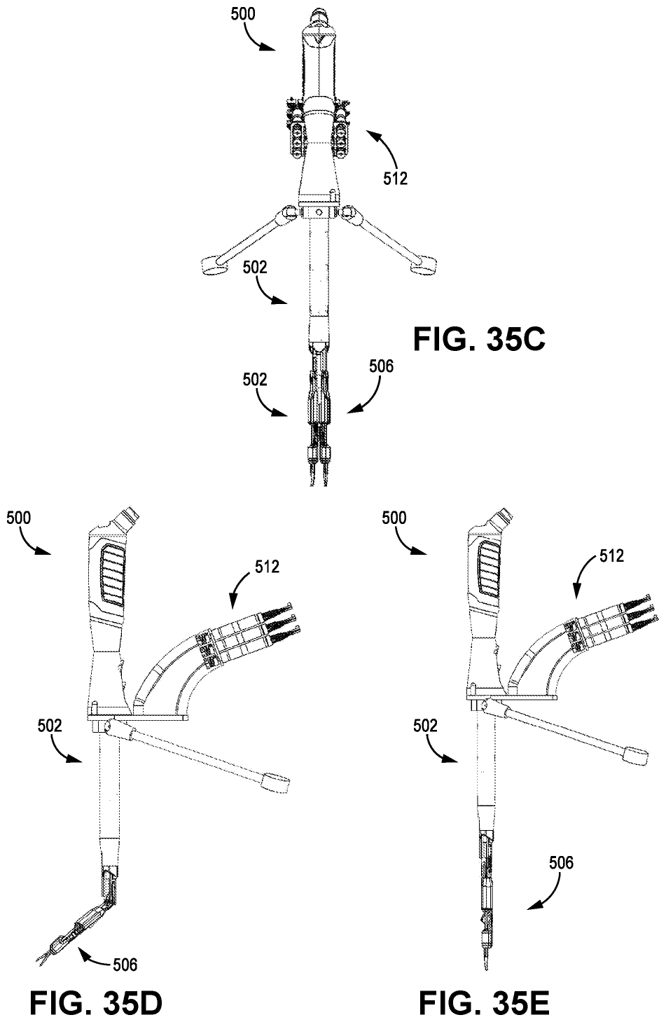

FIG. 50A is a perspective view of the robotic device of FIG. 35A with the camera positioned for insertion into or removed from the device body, according to one embodiment.

FIG. 50B is a perspective view of the device and camera of FIG. 50A with the camera being inserted into or removed from the device body, according to one embodiment.

### DETAILED DESCRIPTION

The various systems and devices disclosed herein relate to devices for use in medical procedures and systems. More specifically, various embodiments relate to various medical devices, including robotic devices and related methods and systems, having at least one robotic arm that can be actuated by one or more motors and related gears disposed within the arm, one or motors and related cables extending from the motor such that the motor can be disposed within any portion of the arm, the device body, or external to the device, or a combination of motors/gears and motors/cables. While the various embodiments herein are generally described in the context of a robotic device having two arms, it is understood that the various actuation assembly embodiments can be incorporated into any arm of any robotic device or system, including devices having solely one arm, three arms, four arms, or more. Further, any of the implementations herein can be incorporated into any type of robotic device or system, including non-surgical devices and systems.

It is understood that the various embodiments of robotic devices and related methods and systems disclosed herein can be incorporated into or used with any other known medical devices, systems, and methods.

It is understood that the various embodiments of robotic devices and related methods and systems disclosed herein can be incorporated into or used with any other known medical devices, systems, and methods. For example, the various embodiments disclosed herein may be incorporated into or used with any of the medical devices and systems disclosed in U.S. Pat. No. 8,968,332 (issued on Mar. 3, 2015 and entitled “Magnetically Coupleable Robotic Devices and Related Methods”), U.S. Pat. No. 8,834,488 (issued on Sep. 16, 2014 and entitled “Magnetically Coupleable Surgical Robotic Devices and Related Methods”), U.S. Pat. No. 10,307,199 (issued on Jun. 4, 2019 and entitled “Robotic Surgical Devices and Related Methods”), U.S. Pat. No. 9,579,088 (issued on Feb. 28, 2017 and entitled “Methods, Systems, and Devices for Surgical Visualization and Device Manipulation”), U.S. Patent Application 61/030,588 (filed on Feb. 22, 2008), U.S. Pat. No. 8,343,171 (issued on Jan. 1, 2013 and entitled “Methods and Systems of Actuation in Robotic Devices”), U.S. Pat. No. 8,828,024 (issued on Sep. 9, 2014 and entitled “Methods and Systems of Actuation in Robotic Devices”), U.S. Pat. No. 9,956,043 (issued on May 1, 2018 and entitled “Methods and Systems of Actuation in Robotic Devices”), U.S. Pat. No. 10,695,137 (issued Jun. 30, 2020 and entitled “Methods and Systems of Actuation in Robotic Devices”), U.S. patent application Ser. No. 16/916,539 (filed on Jun. 30, 2020 and entitled “Methods and Systems of Actuation in Robotic Devices”), U.S. Pat. No. 10,335,024 (issued Jul. 2, 2019 and entitled “Medical Inflation, Attachment, and Delivery Devices and Related Methods”), U.S. Pat. No. 8,974,440 (issued on Mar. 10, 2015 and entitled “Modular and Cooperative Medical Devices and Related Systems and Methods”), U.S. Pat. No. 8,679,096 (issued on Mar. 25, 2014 and entitled “Multifunctional Operational Component for Robotic Devices”), U.S. Pat. No. 9,179,981 (issued on Nov. 10, 2015 and entitled “Multifunctional Operational Component for Robotic Devices”), U.S. Pat. No. 9,883,911 (issued on Feb. 6, 2018 and entitled “Multifunctional Operational Component for Robotic Devices”), U.S. Pat. No. 10,376,323 (issued on Aug. 13, 2019 and entitled “Multifunctional Operational Component for Robotic Devices”), U.S. Pat. No. 10,959,790 (issued on Mar. 30, 2021 and entitled “Multifunctional Operational Component for Robotic Devices”), U.S. patent application Ser. No. 17/217,057 (filed on Mar. 30, 2021 and entitled “Multifunctional Operational Component for Robotic Devices”), U.S. Pat. No. 8,894,633 (issued on Nov. 25, 2014 and entitled “Modular and Cooperative Medical Devices and Related Systems and Methods”), U.S. Pat. No. 8,968,267 (issued on Mar. 3, 2015 and entitled “Methods and Systems for Handling or Delivering Materials for Natural Orifice Surgery”), U.S. Pat. No. 9,060,781 (issued on Jun. 23, 2015 and entitled “Methods, Systems, and Devices Relating to Surgical End Effectors”), U.S. Pat. No. 9,757,187 (issued on Sep. 12, 2017 and entitled “Methods, Systems, and Devices Relating to Surgical End Effectors”), U.S. Pat. No. 10,350,000 (issued on Jul. 16, 2019 and entitled “Methods, systems, and devices relating to surgical end effectors”), U.S. Pat. No. 11,065,050 (issued on Jul. 20, 2021 and entitled “Methods, Systems, and Devices Relating to Surgical End Effectors”), U.S. patent application Ser. No. 17/352,817 (filed on Jun. 21, 2021 and entitled “Methods, Systems, and Devices Relating to Surgical End Effectors”), U.S. Pat. No. 9,089,353 (issued on Jul. 28, 2015 and entitled “Robotic Surgical Devices, Systems, and Related Methods”), U.S. Pat. No. 10,111,711 (issued on Oct. 30, 2018 and entitled “Robotic Surgical Devices, Systems, and Related Methods”), U.S. Pat. No. 11,032,125 (issued on Jun. 8, 2021 and entitled “Robotic Surgical Devices, Systems, and Related Methods”), U.S. patent application Ser. No. 17/340,669 (filed on Jun. 7, 2021 and entitled “Robotic Surgical Devices, Systems and Related Methods”), U.S. Pat. No. 9,770,305 (issued on Sep. 26, 2017 and entitled “Robotic Surgical Devices, Systems, and Related Methods”), U.S. Pat. No. 10,624,704 (issued on Apr. 21, 2020 and entitled “Robotic Surgical Devices, Systems, and Related Methods”), U.S. Pat. No. 11,051,895 (issued on Jul. 6, 2021 and entitled “Robotic Surgical Devices, Systems, and Related Methods”), U.S. patent application Ser. No. 17/368,023 (filed on Jul. 6, 2021 and entitled “Robotic Surgical Devices, Systems, and Related Methods”), U.S. Pat. No. 10,582,973 (issued on Mar. 10, 2020 and entitled “Robotic Surgical Devices, Systems, and Related Methods”), U.S. patent application Ser. No. 16/814,223 (filed on Mar. 10, 2020 and entitled “Robotic Surgical Devices, Systems, and Related Methods”), U.S. patent application Ser. No. 13/738,706 (filed on Jan. 10, 2013 and entitled “Methods, Systems, and Devices for Surgical Access and Insertion”), U.S. patent application Ser. No. 14/661,465 (filed on Mar. 18, 2015 and entitled “Methods, Systems, and Devices for Surgical Access and Insertion”), U.S. patent application Ser. No. 15/890,860 (filed on Feb. 7, 2018 and entitled “Methods, Systems, and Devices for Surgical Access and Insertion”), U.S. patent application Ser. No. 16/999,407 (filed on Aug. 21, 2020 and entitled “Methods, Systems, and Devices for Surgical Access and Insertion”), U.S. Pat. No. 9,498,292 (issued on Nov. 22, 2016 and entitled “Single Site Robotic Devices and Related Systems and Methods”), U.S. Pat. No. 10,219,870 (issued on Mar. 5, 2019 and entitled “Single site robotic device and related systems and methods”), U.S. patent application Ser. No. 16/293,135 (filed Mar. 3, 2019 and entitled “Single Site Robotic Device and Related Systems and Methods”), U.S. Pat. No. 9,010,214 (issued on Apr. 21, 2015 and entitled “Local Control Robotic Surgical Devices and Related Methods”), U.S. Pat. No. 10,470,828 (issued on Nov. 12, 2019 and entitled “Local Control Robotic Surgical Devices and Related Methods”), U.S. patent application Ser. No. 16/596,034 (filed on Oct. 8, 2019 and entitled “Local Control Robotic Surgical Devices and Related Methods”), U.S. Pat. No. 9,743,987 (issued on Aug. 29, 2017 and entitled “Methods, Systems, and Devices Relating to Robotic Surgical Devices, End Effectors, and Controllers”), U.S. Pat. No. 10,603,121 (issued on Mar. 31, 2020 and entitled “Methods, Systems, and Devices Relating to Robotic Surgical Devices, End Effectors, and Controllers”), U.S. patent application Ser. No. 16/834,131 (filed on Mar. 30, 2020 and entitled “Methods, Systems, and Devices Relating to Robotic Surgical Devices, End Effectors, and Controllers”), U.S. Pat. No. 9,888,966 (issued on Feb. 13, 2018 and entitled “Methods, Systems, and Devices Relating to Force Control Surgical Systems”), U.S. Pat. No. 10,743,949 (issued on Aug. 18, 2020 and entitled “Methods, Systems, and Devices Relating to Force Control Surgical Systems”), U.S. patent application Ser. No. 16/922,560 (filed on Jul. 7, 2020 and entitled “Methods, Systems, and Devices Relating to Force Control Surgical Systems”), U.S. Pat. No. 10,667,883 (issued on Jun. 2, 2020 and entitled “Robotic Surgical Devices, Systems, and Related Methods”), U.S. patent application Ser. No. 16/890,424 (filed on Jun. 2, 2020 and entitled “Robotic Surgical Devices, Systems, and Related Methods”), U.S. Pat. No. 10,966,700 (issued on Apr. 6, 20201 and entitled “Robotic Surgical Devices, Systems, and Related Methods”), U.S. patent application Ser. No. 17/147,172 (filed on Jan. 12, 2021 and entitled “Robotic Surgical Devices, Systems, and Related Methods”), U.S. Pat. No. 10,342,561 (issued on Jul. 9, 2019 and entitled “Quick-Release End Effectors and Related Systems and Methods”), U.S. patent application Ser. No. 16/504,793 (filed on Jul. 8, 2019 and entitled “Quick-Release End Effectors and Related Systems and Methods”), U.S. Pat. No. 10,376,322 (issued on Aug. 13, 2019 and entitled “Robotic Device with Compact Joint Design and Related Systems and Methods”), U.S. patent application Ser. No. 16/538,902 (filed on Aug. 13, 2019 and entitled “Robotic Device with Compact Joint Design and Related Systems and Methods”), U.S. Pat. No. 10,806,538 (issued on Oct. 20, 2020 and entitled Robotic Surgical Devices, System and Related Methods”), U.S. patent application Ser. No. 17/075,122 (filed on Oct. 20, 2020 and entitled Robotic Surgical Devices, System and Related Methods”), U.S. Pat. No. 10,751,136 (issued on Aug. 25, 2020 and entitled “Robotic Surgical Devices, Systems, and Related Methods”), U.S. patent application Ser. No. 16/926,025 (filed on Jul. 10, 2020 and entitled “Robotic Surgical Devices, Systems, and Related Methods”), U.S. patent application Ser. No. 15/687,113 (filed on Aug. 25, 2017 and entitled “Quick-Release End Effector Tool Interface”), U.S. Pat. No. 10,702,347 (issued on Jul. 7, 2020 and entitled “Robotic Device with Compact Joint Design and an Additional Degree of Freedom and Related Systems and Methods”), U.S. patent application Ser. No. 16/921,073 (filed on Jul. 6, 2020 and entitled “Robotic Device with Compact Joint Design and an Additional Degree of Freedom and Related Systems and Methods”), U.S. patent application Ser. No. 15/821,169 (filed on Nov. 22, 2017 and entitled “Gross Positioning Device and Related Systems and Methods”), U.S. Pat. No. 10,675,110 (issued on Jun. 9, 2020 and entitled “User Controller with User Presence Detection and Related Systems and Methods”), U.S. patent application Ser. No. 16/896,678 (filed on Jun. 9, 2020 and entitled “User Controller with User Presence Detection and Related Systems and Methods”), U.S. Pat. No. 10,722,319 (issued on Jul. 28, 2020 and entitled “Releasable Attachment Device for Coupling to Medical Devices and Related Systems and Methods”), U.S. patent application Ser. No. 16/904,101 (filed on Jun. 17, 2020 and entitled “Releasable Attachment Device for Coupling to Medical Devices and Related Systems and Methods”), U.S. Pat. No. 11,051,894 (issued on Jul. 6, 2021 and entitled “Robotic Surgical Devices with Tracking Camera Technology and Related Systems and Methods”), U.S. patent application Ser. No. 17/367,915 (filed on Jul. 6, 2021 and entitled “Robotic Surgical Devices with Tracking Camera Technology and Related Systems and Methods”), U.S. Pat. No. 11,013,564 (issued on May 25, 2021 and entitled “Single-Manipulator Robotic Device With Compact Joint Design and Related Systems and Methods”), U.S. patent application Ser. No. 17/236,489 (filed on Apr. 21, 2021 and entitled “Single-Manipulator Robotic Device With Compact Joint Design and Related Systems and Methods”), U.S. patent application Ser. No. 16/736,329 (filed on Jan. 7, 2020 and entitled “Robotically Assisted Surgical System and Related Devices and Methods”), U.S. patent application Ser. No. 17/368,255 (filed on Jul. 6, 2021 and entitled “Surgical Robot Positioning System and Related Devices and Methods”), U.S. Pat. No. 7,492,116 (filed on Oct. 31, 2007 and entitled “Robot for Surgical Applications”), U.S. Pat. No. 7,772,796 (filed on Apr. 3, 2007 and entitled “Robot for Surgical Applications”), and U.S. Pat. No. 8,179,073 (issued on May 15, 2011, and entitled “Robotic Devices with Agent Delivery Components and Related Methods”), all of which are hereby incorporated herein by reference in their entireties.

Certain device and system implementations disclosed herein and in the applications listed above can be positioned within a body cavity of a patient, or a portion of the device can be placed within the body cavity, in combination with an external support component. An “in vivo device” as used herein means any device that can be positioned, operated, or controlled at least in part by a user while being positioned within a body cavity of a patient, including through an incision and/or port disposed within an incision, and further including any device that is coupled to an external support component that is disposed outside the patient's body. It also includes any device positioned substantially against or adjacent to a wall of a body cavity of a patient, any device that is internally actuated (having no external source of motive force), and any device that may be used laparoscopically or endoscopically during a surgical procedure. As used herein, the terms “robot,” and “robotic device” shall refer to any device that can perform a task either automatically or in response to a command.

Certain embodiments provide for insertion of the a device implementation as disclosed herein into the cavity while maintaining sufficient insufflation of the cavity. Further embodiments minimize the physical contact of the surgeon or surgical users with the device during the insertion process. Other implementations enhance the safety of the insertion process for the patient and the device. For example, some embodiments provide visualization of the device as it is being inserted into the patient's cavity to ensure that no damaging contact occurs between the system/device and the patient. In addition, certain embodiments allow for minimization of the incision size/length. Other implementations include devices that can be inserted into the body via an incision or a natural orifice, including devices that can positioned through the incision during use. Further implementations reduce the complexity of the access/insertion procedure and/or the steps required for the procedure. Other embodiments relate to devices that have minimal profiles, minimal size, or are generally minimal in function and appearance to enhance ease of handling and use.

As in manual laparoscopic procedures, a known insufflation system can be used to pump sterile carbon dioxide (or other gas) into the patient's abdominal cavity. This lifts the abdominal wall from the organs and creates space for the robot. In certain implementations, the system has no direct interface with the insufflation system. Alternatively, the various system embodiments herein can have a direct interface to the insufflation system.

In certain implementations in which the device is inserted through an insertion port, the insertion port is a known, commercially-available flexible membrane placed transabdominally to seal and protect the abdominal incision. This off-the-shelf component is the same device or substantially the same device that is used in substantially the same way for Hand-Assisted Laparoscopic Surgery (HALS). The only difference is that the arms of the robotic device according to the various embodiments herein are inserted into the abdominal cavity through the insertion port rather than the surgeon's hand. The robotic device body is disposed within and seals against the insertion port when it is positioned therethrough, thereby maintaining insufflation pressure. The port is single-use and disposable. Alternatively, any known port can be used. In further alternatives, the device can be inserted through an incision without a port or through a natural orifice.

Certain implementations disclosed herein relate to “combination” or “modular” medical devices that can be assembled in a variety of configurations. For purposes of this application, both “combination device” and “modular device” shall mean any medical device having modular or interchangeable components that can be arranged in a variety of different configurations.

Certain embodiments disclosed or contemplated herein can be used for colon resection, a surgical procedure performed to treat patients with lower gastrointestinal diseases such as diverticulitis, Crohn's disease, inflammatory bowel disease and colon cancer. Approximately two-thirds of known colon resection procedures are performed via a completely open surgical procedure involving an 8- to 12-inch incision and up to six weeks of recovery time. Because of the complicated nature of the procedure, existing robot-assisted surgical devices are rarely used for colon resection surgeries, and manual laparoscopic approaches are only used in one-third of cases. In contrast, the various implementations disclosed herein can be used in a minimally invasive approach to a variety of procedures that are typically performed ‘open’ by known technologies, with the potential to improve clinical outcomes and health care costs. Further, the various implementations disclosed herein can be used for any laparoscopic surgical procedure in place of the known mainframe-like laparoscopic surgical robots that reach into the body from outside the patient. That is, the less-invasive robotic systems, methods, and devices disclosed herein feature small, self-contained surgical devices that are inserted in their entireties through a single incision in the patient's abdomen. Designed to utilize existing tools and techniques familiar to surgeons, the devices disclosed herein will not require a dedicated operating room or specialized infrastructure, and, because of their much smaller size, are expected to be significantly less expensive than existing robotic alternatives for laparoscopic surgery. Due to these technological advances, the various embodiments herein could enable a minimally invasive approach to procedures performed in open surgery today.

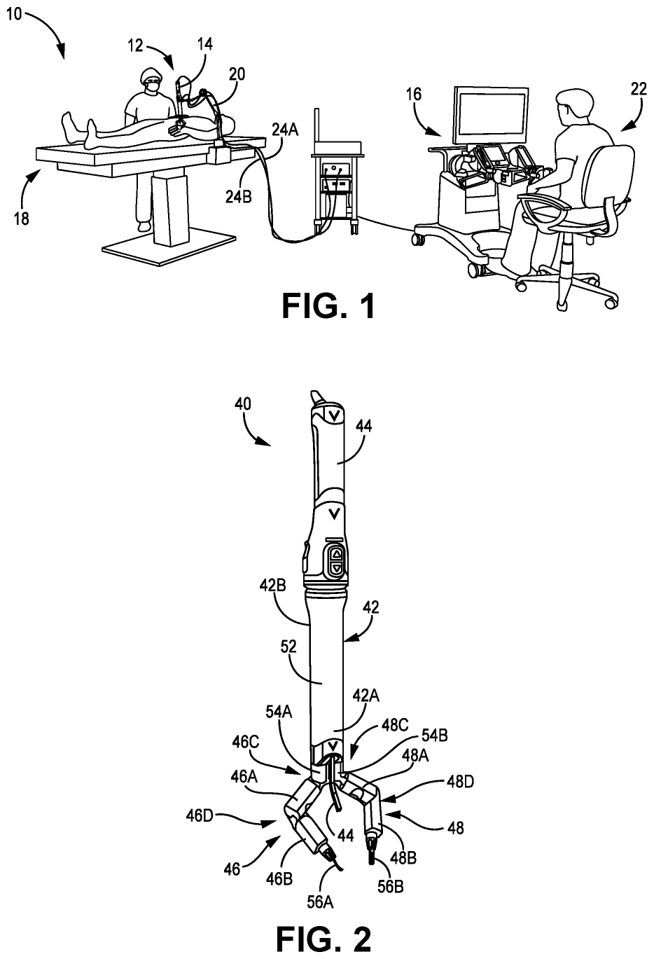

FIG. 1 depicts one embodiment of a robotic surgical system 10 having several components that will be described in additional detail below. The components of the various system implementations disclosed or contemplated herein can include an external control console 16 and a robotic device 12 having a removable camera 14 as will also be described in additional detail below. In accordance with the implementation of FIG. 1, the robotic device 12 is shown mounted to the operating table 18 via a known, commercially available support arm 20. The system 10 can be, in certain implementations, operated by the surgeon 22 at the console 16 and one surgical assistant 24 positioned at the operating table 18. Alternatively, one surgeon 22 can operate the entire system 10. In a further alternative, three or more people can be involved in the operation of the system 10. It is further understood that the surgeon (or user) 22 can be located at a remote location in relation to the operating table 18 such that the surgeon 22 can be in a different city or country or on a different continent from the patient on the operating table 18.

In this specific implementation, the robotic device 12 with the camera 14 are both connected to the surgeon console 16 via cables: a device cable 24A and a camera cable 24B. Alternatively, any connection configuration can be used. In certain implementations, the system can also interact with other devices during use such as a electrosurgical generator, an insertion port, and auxiliary monitors.

FIG. 2 depicts one exemplary implementation of a robotic device 40 that can be incorporated into the exemplary system 10 discussed above or any other system disclosed or contemplated herein. The device 40 has a body (or “torso”) 42 having a distal end 42A and proximal end 42B, with the imaging device (or “camera”) 44 disposed therethrough, as mentioned above and as will be described in additional detail below. Briefly, the robotic device 40 has two anthropometric robotic arms 46, 48 operably coupled to a distal end of the body 42, and the camera 44 is removably positionable through the body 42 and disposed between the two arms 46, 48. That is, device 40 has a first (or “right”) arm 46 and a second (or “left) arm 48, both of which are operably coupled to the body 42 as discussed in additional detail below. In this embodiment, the body 42 of the device 40 as shown has an enclosure (also referred to as a “cover” or “casing”) 52 such that the internal components and lumens of the body 42 are disposed within the enclosure 52. The device body 42 has two rotatable cylindrical bodies (also referred to as “shoulders” or “turrets”) 54A, 54B: a first (or “right”) shoulder 54A and a second (or “left”) shoulder 54B. Each arm 46, 48 in this implementation also has an upper arm (also referred to herein as an “inner arm,” “inner arm assembly,” “inner link,” “inner link assembly,” “upper arm assembly,” “first link,” or “first link assembly”) 46A, 48A, and a forearm (also referred to herein as an “outer arm,” “outer arm assembly,” “outer link,” “outer link assembly,” “forearm assembly,” “second link,” or “second link assembly”) 46B, 48B. The right upper arm 46A is operably coupled to the right shoulder 54A of the body 42 at the right shoulder joint 46C (such that the right shoulder 54A is considered a component of the right shoulder joint 46C) and the left upper arm 48A is operably coupled to the left shoulder 54B of the body 42 at the left shoulder joint 48C (such that the left shoulder 54B is considered a component of the left shoulder joint 48C). Further, for each arm 46, 48, the forearm 46B, 48B is rotatably coupled to the upper arm 46A, 48A at the elbow joint 46D, 48D. In various embodiments, the forearms 46B, 48B are configured to receive various removeable, interchangeable end effectors 56A, 56B.

The end effectors 56A, 56B on the distal end of the arms 46, 48 can be various tools 56A, 56B (scissors, graspers, needle drivers and the like), as will be described in additional detail below. In certain implementations, the tools 56A, 56B are designed to be removable, including in some instances by a small twist of the tool knob that couples the end effector 56A, 56B to the arm 46, 48. In certain implementations, at least two single-use, interchangeable, disposable surgical end effectors can be used with any of the robotic device embodiments herein (including device 40). Such end effectors can include, but are not limited to, a fenestrated grasper capable of bi-polar cautery, scissors that deliver mono-polar cautery, a hook that delivers mono-polar cautery, and a left/right needle driver set. The tools can be selected for the specific surgical task. Certain forearm and end effector configurations that allow for the removability and interchangeability of the end effectors are disclosed in detail in U.S. application Ser. Nos. 16/504,793 and 15/687,113, both of which are incorporated herein (and above) by reference. Further, it is understood that any known forearm and end effector combinations can be used in any of the robotic device embodiments disclosed or contemplated herein.

In various implementations, the body 40 and each of the links of the arms 46, 48 can contain a variety of actuators and/or motors, as will be described in additional detail below. In certain implementations, the body 40 has no motors disposed therein, while there is at least one motor in each of the arms 46, 48. Alternatively, the body 40 can have at least one motor disposed therein, while the each of the arms 46, 48 has zero, one, two, three, four, or more motors disposed therein. In a further alternative, the various components of any device implementation herein can have any configuration of motors or no motors therein. In one embodiment, any of the motors discussed and depicted herein can be brush or brushless motors. Further, the motors can be, for example, 6 mm, 8 mm, or 10 mm diameter motors. Alternatively, any known size that can be integrated into a medical device can be used. In a further alternative, the actuators can be any known actuators used in medical devices to actuate movement or action of a component. Examples of motors that could be used for the motors described herein include the EC 10 BLDC+GP10A Planetary Gearhead, EC 8 BLDC+GP8A Planetary Gearhead, or EC 6 BLDC+GP6A Planetary Gearhead, all of which are commercially available from Maxon Motors, located in Fall River, Mass. There are many ways to actuate these motions, such as with DC motors, AC motors, permanent magnet DC motors, brushless motors, pneumatics, cables coupled to motors, hydraulics, and the like. As such, the actuation source can be at least one motor, hydraulic pressure source, pneumatic pressure source, or any other actuation source disposed remotely from or proximally to the device 40 such that an appropriate coupling or transmission mechanism (such as at least one cable, at least one hydraulic transmission hose, at least one pneumatic transmission hose, or any other transmission mechanism) is disposed through the body 42.

In one embodiment, the various joints discussed above in accordance with any of the embodiments disclosed or contemplated herein can be driven by electrical motors disposed within the device and, in some implementations, near each joint. Other embodiments include the incorporation of pneumatic or hydraulic actuators in any of the device implementations herein. In additional alternative embodiments, the driving actuators are disposed within the device body or outside the device and/or body cavity and power transmission mechanisms are provided to transmit the energy from the external source to the various joints of any device herein. Such a transmission mechanism could, for example, take the form of gears, drive shafts, cables, pulleys, or other known mechanisms, or any combination thereof.

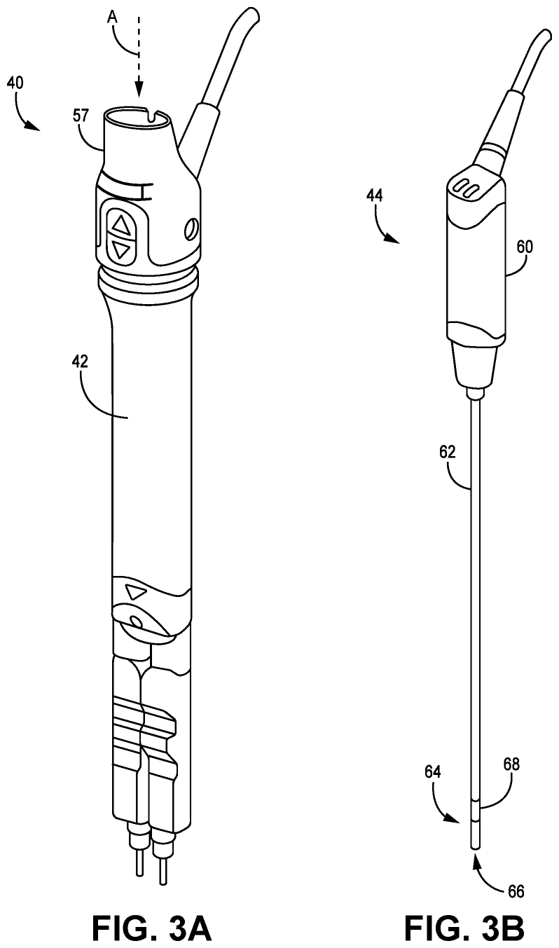

FIGS. 3A and 3B depict one embodiment of the robotic device 40 with the camera assembly 44 removed, according to one implementation. That is, FIG. 3A depicts the device 40 without the camera positioned through the body 42, and FIG. 3B depicts one embodiment of the camera 44. In certain implementations, and as best shown in FIG. 3B, the camera 44 has a handle (or “camera body”) 60 with an elongate shaft 62 coupled thereto such that the shaft 62 extends distally from the distal end of the handle 60. In addition, the camera 44 has a steerable tip 64 coupled to the distal end of the shaft 62 via a flexible section 68 such that the steerability allows the user to adjust the viewing direction, as will be discussed in further detail below. Further, the tip 64 also includes a camera imager 66 at the distal end of the tip 64 that is configured to capture the desired images. Further, the tip 64 in certain implementations has an illumination light (not shown) disposed thereon, such that the light can illuminate the objects in the field of view. In one specific implementation, the camera 44 provides 1080p 60 Hz. digital video. Alternatively, the camera 44 can provide any known video quality.

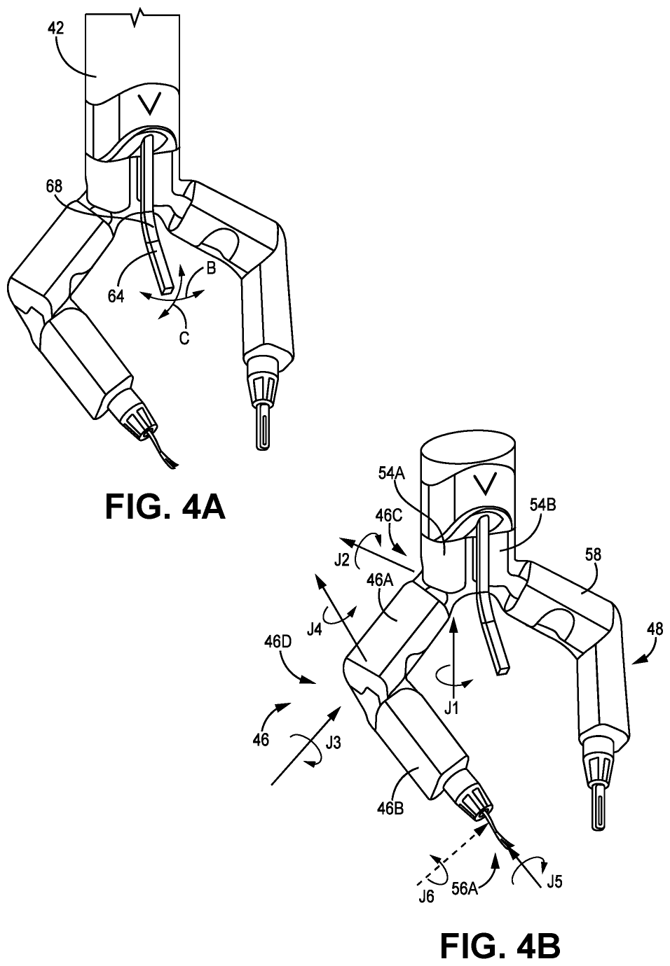

As best shown in FIGS. 3A and 4A, the camera assembly 44 can be inserted into the body 42 of the robotic device 40 by positioning the distal end of the shaft 62 through a lumen (not shown) defined through the body 42 of the robotic device 40 as shown by the arrow A in FIG. 3A. As will be described in further detail below, certain implementations of the device 40 include a removable nest (or “dock”) 57 disposed near the proximal end of the body 42 that includes a seal (not shown) that operates to ensure that the patient's cavity remains insufflated. That is, the seal (not shown) makes it possible to remove the camera 44 from the body 42 while maintaining insufflation (similar to a manual laparoscopic port). The nest 57 can also contain a connection or locking mechanism (not shown) that locks the camera 44 into the device body 42 until the camera latch button 59 is pressed to release the locking mechanism and thereby allow for removal of the camera 44.

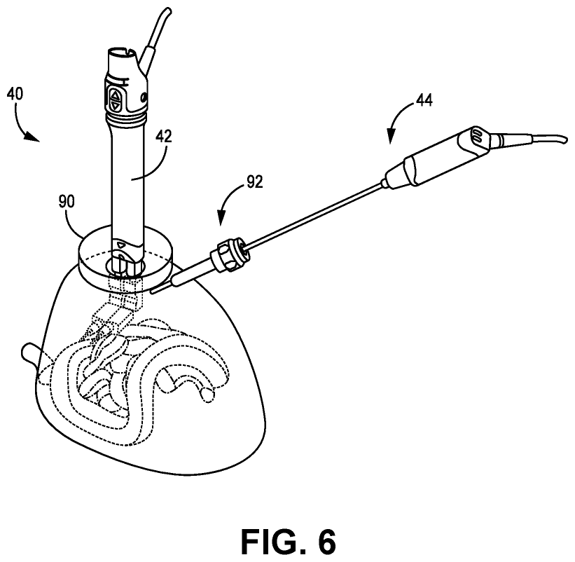

In accordance to certain embodiments, at least one or both of the nest 57 and device body 42 contain sensors (not shown) configured to indicate when the camera assembly 44 is properly disposed within the nest 57 and device body 42 and locked in place. In such implementations, the device 40 is inoperable unless the camera 44 properly locked in place. In such implementations and in some alternative embodiments, the device 40 is also inoperable for purposes of insertion or extraction unless the camera 44 has been removed (so it can be placed in an auxiliary port such as the port 92 as shown in FIG. 6 and discussed in additional detail below). Easy camera 44 removal also facilitates cleaning of the lens and other parts of the camera 44 to remove any debris that may be generated during surgery.

According to some implementations, the nest 57, and specifically the locking mechanism, allow for the device 40 and the camera 44 locked therein to move together, thereby resulting in the camera 44 being positioned to provide constant visualization of the arms 46, 48 and end effectors 56A, 56B while always maintaining proper triangulation between the device 40, camera 44, and arms 46, 48/end effectors 56A, 56B. That is, it ensures that the camera 44 is always well positioned with respect to the arms 46, 48 and end effectors 56A, 56B and the configuration does not change during operation of the device 40.

When the shaft 62 is inserted through the lumen of the body 42 as desired, according to certain embodiments as best shown in FIGS. 2 and 4A, the distal end of the shaft 62, including the flexible section 68 and the steerable tip 64 (containing the imager 66) extends out of an opening at the distal end of the body 42 such that the tip 64 is positioned between the two arms 46, 48 in the surgical environment as shown. Thus, the imager 66 is positioned to capture the view between the two arms 46, 48 and the steerable tip 64 can be actuated to provide views of the surgical tools and surgical target. That is, the tip 64 can be moved such that the surgical tools and/or surgical target are captured within the field of view of the imager 66. It is understood that this camera 44 embodiment and any other such camera embodiment disclosed or contemplated herein can be used with any similar robotic device having a camera lumen defined therethrough.

In various implementations, as best shown in FIG. 4A, the steerable tip 64 and therefore also the camera imager 66 can be steered or otherwise moved in two independent directions in relation to the shaft 62 at a flexible section 68 disposed between the shaft 62 and the steerable tip 64 to change the direction of view. That is, FIG. 4A shows that the steerable tip 64 can be robotically articulated in the yaw direction (left and right in relation to the device 40) as represented by arrow B or pitch direction (up and down in relation to the device 40) as represented by arrow C. In various implementations, the camera 44 can be controlled via a console (such as console 16 discussed above, for example) or via control buttons (not shown) as will be discussed in additional detail below. In one embodiment, the features and operation (including articulation) of the steerable tip are substantially similar to the steerable tip as described in U.S. application Ser. Nos. 14,334,383 and 15/227,813, both of which are incorporated by reference above, and any other applications incorporated by reference above disclosing such steerable tips. Alternatively, any known robotic articulation mechanism for cameras or similar apparatuses can be incorporated into any camera embodiment utilized in any device or system disclosed or contemplated herein.

In various implementations, the camera 44 can be re-sterilized for multiple uses. In one specific embodiment, the camera 44 can be reused up to one hundred times or more. Alternatively, it is understood that any known endoscopic camera that can fit through a device body according to any implementation herein can be utilized.

Focusing now on the robotic arms 46, 48 of the robotic device 40 according to one embodiment as shown in FIGS. 4A-4B, each robot arm 46, 48 in this implementation has six degrees of freedom, including the open/close function of the tool, as best shown in FIG. 4B. For purposes of this discussion, the various degrees of freedom will be discussed in the context of the right arm 46 as shown in FIG. 4B, but it is understood that both arms have the same degrees of freedom. The right shoulder joint 46C is approximately a spherical joint similar to a human shoulder. The upper arm 46A can yaw (J1), pitch (J2), and roll about the shoulder joint 46C (J3). These first three axes of rotation roughly intersect at the shoulder joint 46C. The robot elbow 46D (J4) allows rotation of the forearm 46B with respect to the upper arm 46A. Finally, the end effector 56A can roll (J5) about the long axis of the end effector 56A and some tools that can be replaceably attached to the forearm 46B have an open/close actuation function. On the other hand, it is understood that a hook cautery tool, for example, does not open/close.

As can be seen in FIG. 4B, the arms 46, 48 in this exemplary implementation have a molded silicon protective sleeve 58 that is disposed over the arms 46, 48 and shoulder turrets 54A, 54B. In one embodiment, the sleeve 58 is fluidically sealed such that it protects the arms 46, 48 and the robotic device 40 from fluid ingress and also helps to simplify post-surgery cleaning and sterilization. The fluidically sealed sleeve 58 is substantially similar to any of the sleeve embodiments disclosed or contemplated in U.S. application Ser. Nos. 14/334,383, 15/227,813, and 16/144,807, all of which are incorporated by reference above, and any other applications incorporated by reference above having other sleeve implementations.

The sleeve 58 and the other fluidically sealed components of the device 40 allow for the device 40 to be submersible in 1 meter of water, according to one embodiment.

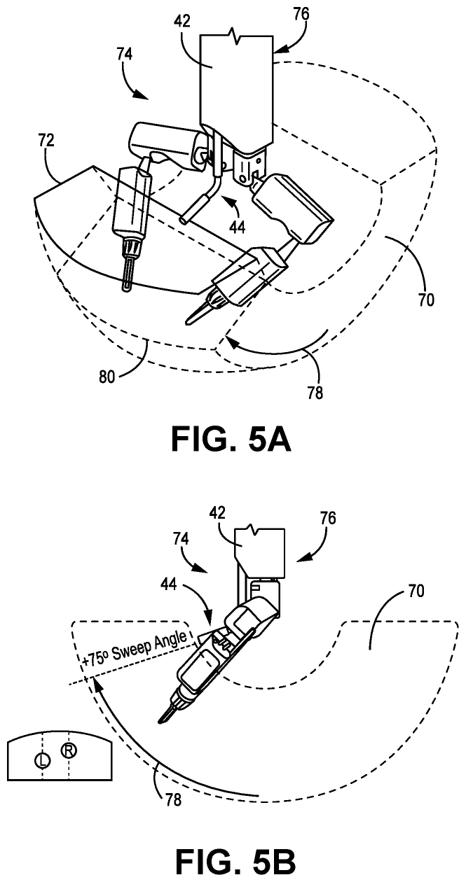

The robotic arms 46, 48 in this implementation have significant dexterity that enables the arms 46, 48 to reach into confined spaces within the target cavity of the patient, such as the abdominal cavity. As shown in FIGS. 5A and 5B, the six degrees of freedom described above allow the arms 46, 48 to reach into the confined spaces of the abdominal cavity. More specifically, FIGS. 5A and 5B schematically depict the entire workspace 70 of the arms 46, 48 of the robotic device 40, according to certain implementations. In these implementations, “workspace” 70 means the space 70 around the robotic device 40 in which either arm 46, 48 (and/or end effector thereof) can move, access, and perform its function within that space 70. In other words, the workspace 110 is the volume that can be reached by at least one of the right and left arms 46, 48. The bi-manual workspace 110 is approximated by an ellipse that is rotated 180 degrees about the shoulder pitch joint (J2 in FIG. 4B). According to one embodiment, the arms 46, 48 herein are substantially the same as or similar to the arms, the degrees of freedom, and the overall workspace and the individual workspaces of each arm as disclosed in U.S. application Ser. No. 17/367,915 and/or U.S. application Ser. No. 16,736,329, both of which are incorporated by reference above.

FIG. 5A depicts a perspective view of the device 40 and further schematically shows the collective workspace 70 of the first and second arms 46, 48 and the cross-section 72 thereof, while FIG. 5B depicts a side view of the device 40 and workspace 70. Note that the each arm 46, 48 has a range of motion and corresponding workspace that extends from the front 74 of the device 40 to the back 76 of the device 40. Thus, the first arm 46 moves equally to the front 74 and the back 76, through about 180° of space relative to the axis of the device body 42 for each arm 46, 48. This overall workspace 70, which constitutes an intersecting or collective workspace 70 based on the separate workspaces of the two arms 46, 48, allows the robotic device 40 to work to the front 74 and back 76 equally well without having to reposition the body 42. That is, the bi-manual workspace 70 extends from in front 74 of the robotic device 40 to below the device 40 and is also behind the back 76 of the device 40 as shown. Thus, the workspace 70 represents a region that is reachable by both the left and right arms 46, 48 and is defined as the bi-manual robot workspace 70. The arms 46, 48 function equally over any sweep angle from +90° to −90° as shown in FIG. 5B, where the workspace 70 represents the full range of sweep angles. In other words, the surgeon will have full robot dexterity when working in this bi-manual region 70.

The workspace cross section 72 as best shown in FIG. 5A is a rectangle with an arched top. As can be seen in the figures, this cross section 72 extends 180 degrees around the shoulder pitch (J2 in FIG. 4B). In accordance with one specific, non-limiting embodiment, the workspace cross section 72 can be about 5 inches (13 cm) wide and about 2.5 inches (6.75 cm) deep. Alternatively, the specific dimensions can vary accordingly to the size of the device (and its components) and the size of the target space.

In contrast, the camera sweep angle range 78 (the range that the camera 44 can move along the same path as the workspace sweep angle as shown in FIG. 5B) is more limited in comparison to the sweep angle range of the robotic arms 46, 48 (as represented by the workspace 70). More specifically, the camera 44 can sweep between +75° to −75°, which is a sufficient range to ensure that the camera can capture the arms 46, 48 and their end effectors 56A, 56B at any position throughout the full workspace 70, thereby ensuring that the user(s) can visualize the instruments. The camera sweep defines a working camera plane 80 (the horizontal midline of the surgical view), as shown in FIG. 5A.

During the procedure, the device 40 can be easily repositioned by moving the device body 42, thus allowing access to different portions of the target cavity (such as the abdominal cavity). In certain implementations, the device body 42 can be moved quickly (in less than 10 seconds in some examples) and easily by adjusting the external support arm (such as arm 20 discussed above). The ability to change the overall position of the device 40 combined with the reach and dexterity of the arms 46, 48 enable a surgeon to work anywhere in the target cavity.

Additional features and components of the robotic device include those disclosed in U.S. application Ser. Nos. 17/147,172, 17/075,122, 17,367,915, 17/236,489, and 16/736,329, all of which are incorporated by reference above, along with all of the other patents and applications incorporated by reference above. It is understood that any robotic device embodiment disclosed or contemplated herein (including, for example, the robotic devices 12, 40, 80 discussed above), can be incorporated into not only the system embodiments disclosed herein, but any other known robotic surgical system. It is further understood that, according to certain implementations, any robotic device disclosed or contemplated herein can be configured such that it can be cleaned and sterilized for multiple uses. In some embodiments, the device can be reused up to ten times or more.

In certain alternative implementations as shown in FIG. 6, the camera 44 can be removed from the robotic device 40 and positioned through another, known laparoscopic port 92 typically used with a standard manual laparoscope. As such, in this embodiment, the device 40 is disposed through a main port (also known as an “insertion port”) 90 and the camera 44 is positioned through the known laparoscopic port 92 as shown. It is understood that this arrangement may be useful to visualize the robotic device 40 to ensure safe insertion and extraction via the main port 90. According to various embodiments, the camera 44 can also be removed from the robotic device 40 so the optics can be cleaned, the camera 44 can be repaired, or for any other reason in which it is beneficial to remove the camera 44. It is understood that while the device 40 and camera 44 are depicted and discussed herein, any device or camera according to any implementation disclosed or contemplated herein can also be used in a similar arrangement and any such camera can also be removed from the device for any reason as discussed herein.

It is understood that the insertion port 90 also can represent the port 90 through which any robotic device embodiment disclosed or contemplated herein is positioned for any procedure as contemplated herein (including those procedures in which the camera 44 is disposed through the device 40). In one embodiment, the insertion port 90 can be a single use commercially available flexible membrane disposed transabdominally to seal and protect the abdominal incision and allow for positioning the body 42 of the device 40 therethrough. In specific implementations, the insertion port 90 is the same device used in Hand-Assisted Laparoscopic Surgery (HALS), including the exemplary port 90 depicted in FIG. 6, which, according to one embodiment, is a GelPort™ 90. The device body 42 seals against the insertion port 90, thereby establishing a fluidic seal and thus maintaining insufflation pressure. Alternatively, any known insertion port (or incision) that is configured to receive a device similar to that disclosed herein can be used.

The various implementations herein are devices having one or more robotic arms with specific actuation configurations. More specifically, while some of the embodiments relate to one or more robotic arms actuated by motors and gears that translate the motive force from the motors to the moveable arm components, other implementations have one or more arms that are actuated by motors and cables that translate the motive force from the motors to the moveable arm components. Further, additional embodiments relate to devices having one or more arms that are actuated by a combination of (1) motors and gears and (2) motors and cables. In the various implementations having at least one or more motor and cable configuration, the motor can be disposed anywhere in relation to the moveable component. That is, the motor can be disposed within the same arm component as the moveable component (such as the forearm), in the adjacent component (such as the upper arm), in the device body, or at an external location in relation to the device. Further details about these various embodiments are described in further detail below.

In any of the embodiments disclosed in additional detail below in which one or more cables is used, the cable(s) can be a standard pull cable using an opposing cable for restoration force (or a spring or other method for restoration force), a push/pull cable, Bowden cables, rotary torque transmission cables, or any other known cables for use in medical or robotic devices.

Further, there are many ways to actuate these motions, such as with pneumatics, hydraulics, and the like.

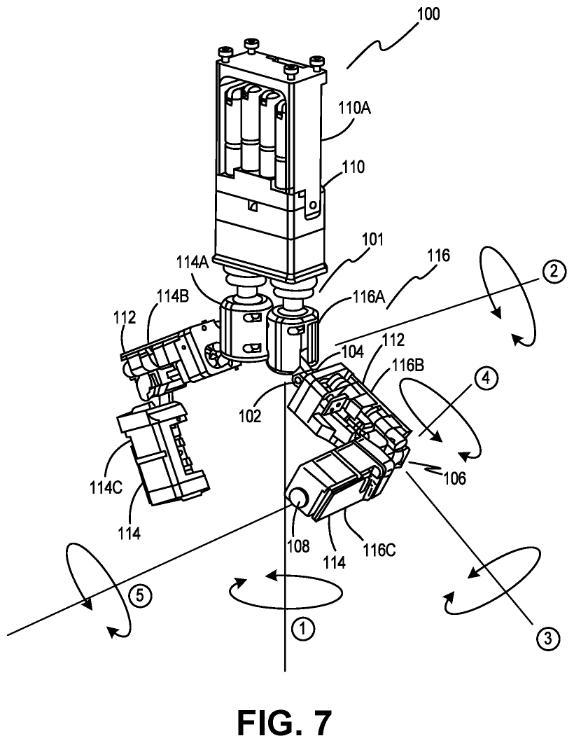

As mentioned above, certain device embodiments have at least one robotic arm actuated by motors that use gear drives to create motion at each joint. One such implementation is set forth in FIGS. 7-20 herein, which depict the internal components of the body 110A and arms 114, 116 of the device 100, which are shown in these figures without their casings or housings. It is understood that in use, these implementations are covered by a housing, in a similar fashion as the embodiment depicted in FIGS. 2-6. FIGS. 7-20 include the internal structural/support components and the actuation (motor and gear) components of the device 100. It is further understood that any of the motor and gear configurations as set forth in U.S. application Ser. Nos. 16/890,424, 17/147,172, 17/075,122, 16/926,025, 17/367,915, 17/236,489, and 16/736,329, all of which are incorporated by reference above, can also be incorporated into any of the devices herein. In use, the various motors used to actuate the robot 100 and its associated components can include, but are not limited to, DC motors, AC motors, Permanent magnet DC motors, brushless motors, and the like. In one embodiment, any of the motors discussed and depicted herein can be brush or brushless motors. Further, the motors can be, for example, 6 mm, 8 mm, or 10 mm diameter motors. Alternatively, any known size that can be integrated into a medical device can be used. In a further alternative, the actuators can be any known actuators used in medical devices to actuate movement or action of a component. Examples of motors that could be used for the motors described herein include the EC 10 BLDC+GP10A Planetary Gearhead, EC 8 BLDC+GP8A Planetary Gearhead, or EC 6 BLDC+GP6A Planetary Gearhead, all of which are commercially available from Maxon Motors, located in Fall River, Mass.

FIG. 7, according to one embodiment, shows an implementation of the robot 100 and each joint of one arm—here, the left arm 116. It is understood that the right arm 114 of this implementation is a mirror image of the left 116 and that the internal components in the left arm 116 that operate/control/actuate the left arm 116 are substantially the same as those depicted and described herein and that the descriptions provided below apply equally to those components as well. Alternatively, the device 100 can have only one arm.

As shown in FIG. 7, the shoulder joints 114A, 116A have a shoulder yaw joint 101 and a shoulder pitch joint 102. In these implementations, an upper arm roll joint 104, an elbow joint 106, and a tool roll joint 108 are also provided which enable a substantial range of motion. In various implementations, a tool actuation joint (not shown) interfaces with the tool (not shown) to actuate open and close of the tool, as has been previously described.

In various implementations, these joints 101, 102, 104, 106 have practical defined ranges of motions that, together with the robot geometry, lead to the final workspace of the robot 100 similar to the workspace discussed above. For the examples given herein, the joint limits allow for a significant robot workspace, as is described above. This workspace allows the various implementations of the robot to use both arms and hands effectively in several locations within the body cavity of the patient.

In the implementation of FIG. 7, the body 110A and each link (meaning the upper arm 116B, and forearm 1160) contain Printed Circuit Boards (“PCBs”) 110, 112, 114 that have embedded sensor, amplification, and control electronics. One PCB is in each forearm and upper arm and two PCBs are in the body. Each PCB also has a full 6 axis accelerometer-based Inertial Measurement Unit and temperature sensors that can be used to monitor the temperature of the motors. Alternatively, any known processors can be used. Each joint can also have either an absolute position sensor or an incremental position sensor or both. In certain implementations, the some joints contain both absolute position sensors (magnetic encoders) and incremental sensors (hall effect). In other implementations, certain joints only have incremental sensors. These sensors are used for motor control. The joints could also contain many other types of sensors. A more detailed description of one possible method is included here.

In this implementation, a larger PCB 110 is mounted to the posterior side of the body 110A. This body PCB 110 controls the motors 116 in the base link, or body 110A (the shoulder yaw joint 101 and shoulder pitch joint 102 for left and right arms, respectively). Each upper arm has a PCB 112 to control the upper arm roll joint 104 and elbow joint 106. Each forearm has a PCB 114 to control the tool roll joint 108 and tool actuation joint (not shown). In the implementation of FIG. 14, each PCB 110, 112, 114 also has a full six axis accelerometer-based inertial measurement unit and several temperature sensors that can be used to monitor the temperature of the various motors described herein.

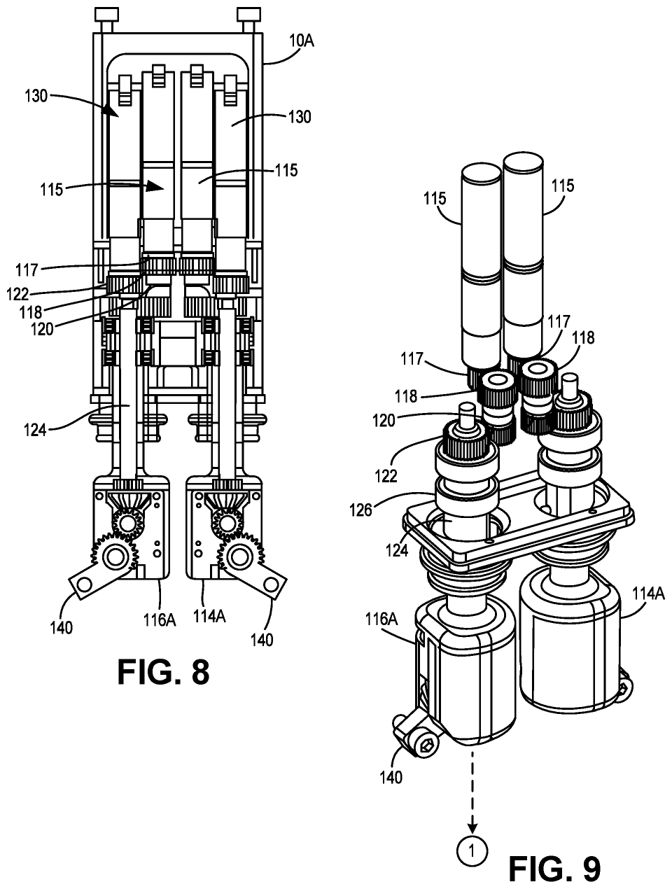

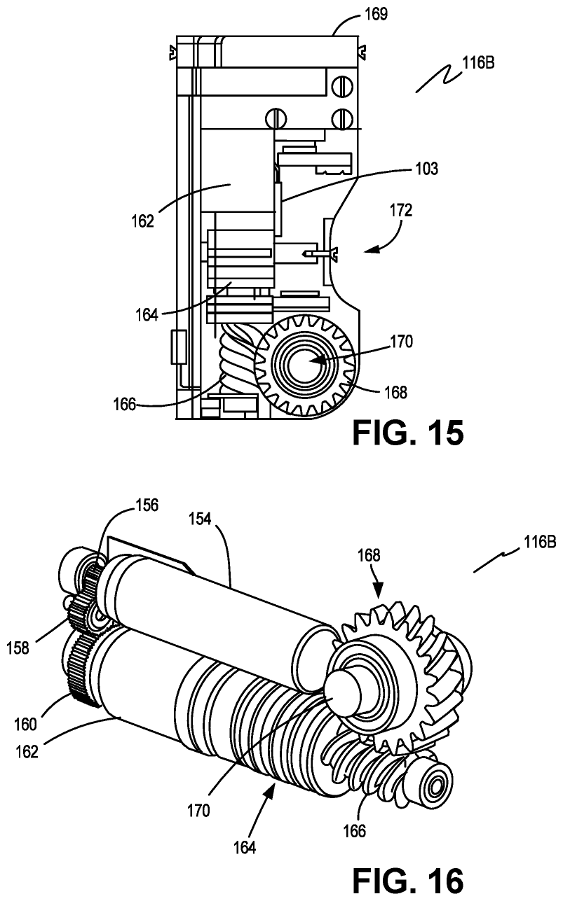

In these embodiments, each joint 101, 102, 104, 106, 108 can also have either an absolute position sensor or an incremental position sensor or both, as described and otherwise disclosed in U.S. application Ser. Nos. 17/368,023 and 16/814,223, which are incorporated by reference above, and any other such sensors as described in any other applications incorporated by reference above. Further, in certain implementations, and as shown in FIG. 8 and elsewhere, any of the various actuators or motors 115, 130, 154, 178 in any of the embodiments described herein can have at least one temperature sensor 103 disposed on the surface of the motor, for example by temperature-sensitive epoxy, such that the temperature sensor 103 (as shown in FIG. 15) can collect temperature information from each actuator for transmission to the control unit, as discussed below.

In this implementation, joints 1-4 have both absolute position sensors (magnetic encoders) and incremental sensors (hall effect). Joints 5 & 6 only have incremental sensors, according to one embodiment. These sensors are used for motor control. It is understood that the joints could also contain many other types of sensors, as have been described in detail in the incorporated applications and references. In a further alternative, any combination of these sensors can be used, or no sensors can be used.

According to one implementation, certain other internal components depicted in the implementation of FIGS. 8 and 9 are configured to actuate the rotation of the shoulder yaw joint 101 of the body 110A around axis 1, as shown in FIG. 7. As mentioned above, it is understood that two of each of the described components are used—one for each arm—but for ease of description, in certain depictions and descriptions, only one is used.

As best shown in FIG. 9, a shoulder yaw joint 101 motor 115 and gearhead combination drives a motor gear 117 first gear set 118, which is best shown in FIG. 16. The first gear set 118 drives a shaft supported by bearings 120 to drive a second gear set 122. In turn, this second gear set 122 drives an output shaft 124 that is also supported by bearings 126. This output shaft 124 then drives a turret 114A, 116A (representing the shoulder of the robot 100) such that the shoulder 116A rotates around axis 1, as best shown in FIG. 7. The various gears herein can be spur gears. Alternatively, any of the gears can be any known type of gear.

As will be discussed in further detail below, in certain alternative embodiments, the motors 115, 130 (and all the motors discussed elsewhere herein with respect to the various implementations), can be placed in more proximal locations if various other shafts, pulleys, cables, and/or gears are included.

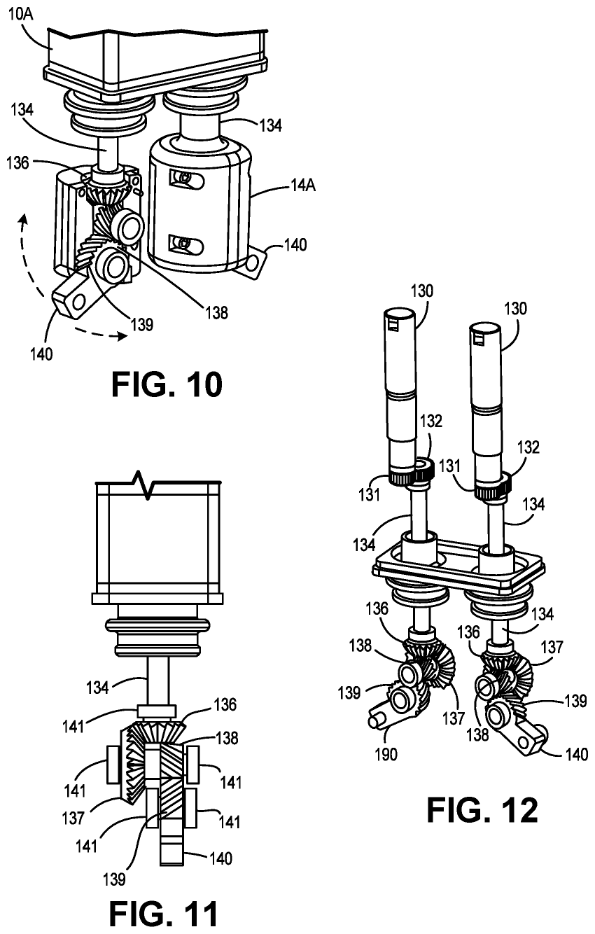

According to one implementation, certain internal components depicted in the implementation of FIGS. 10-12 are configured to actuate the shoulder pitch joint 102 of the body 110A and/or shoulder 114A, 116A around axis 2, as is shown in FIG. 7. In these implementations, the pitch joint 102 is constructed and arranged to pivot the output link 140 so as to move the upper arm (not shown) relative to the shoulder 114A, 116A.

In this specific implementation, as best shown in FIG. 12, a motor 130 and gearhead combination drives a drive gear 131 and driven gear 132 that in turn drives a first shaft 134. This shaft 134 then drives a shoulder gear pair 136, 137 inside the shoulder turret. The shoulder gear pair 136, 137 accordingly drives a driven shoulder gear set 138, 139 directly connected to the shoulder pitch joint 102 output link 140, such that the upper arm 116B rotates around axis 2, as best shown in FIG. 7. In this implementation, the shoulder yaw joint 101 and the shoulder pitch joint 102 therefore have coupled motion. In these implementations, a plurality of bearings 141 support the various gears and other components, as has been previously described. The various gears herein can be spur gears, miter gears, bevel gears, or any other known type of gear.

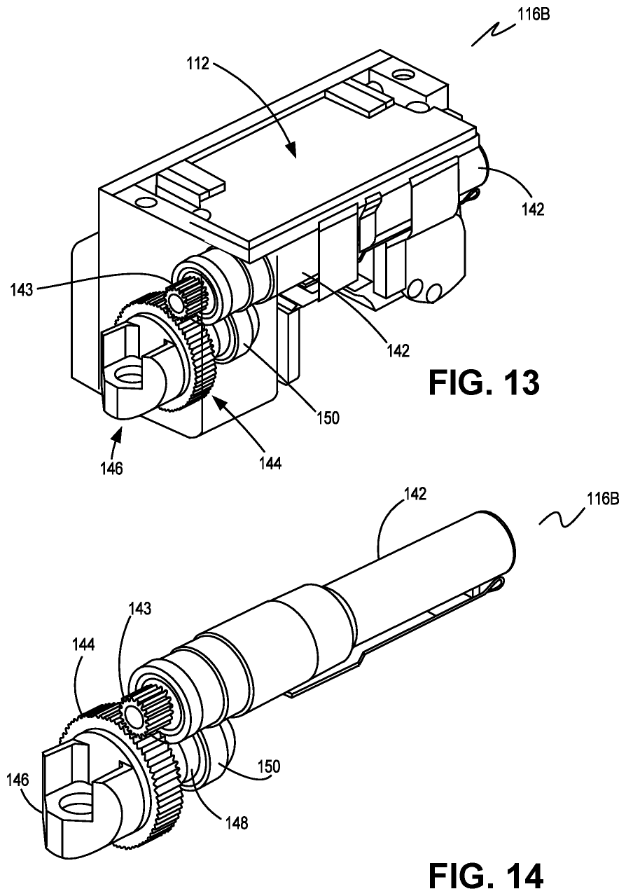

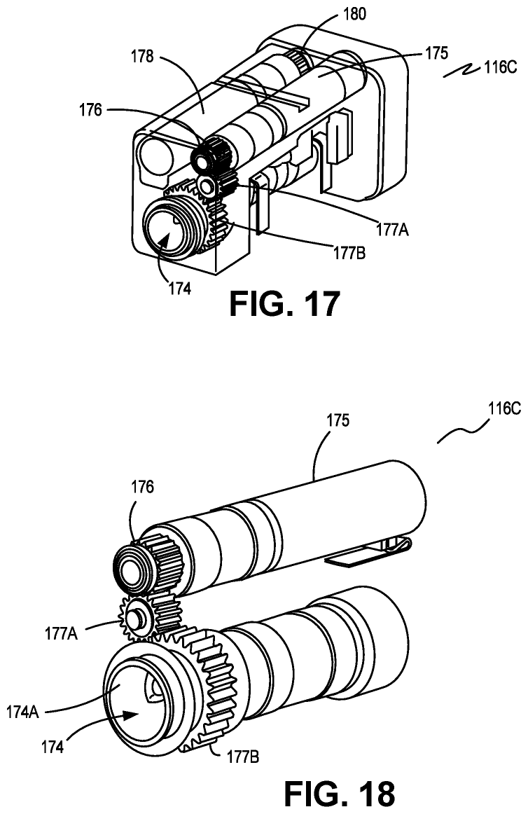

FIGS. 13-16 depict various internal components of the upper arm 116B constructed and arranged for the movement and operation of the arm 116. In various implementations, multiple actuators or motors 142, 154 are disposed within the housing (not shown) of the forearm 116C. FIGS. 17-20 depict various internal components of the forearm 116C constructed and arranged for the movement and operation of the end effectors. In various implementations, multiple actuators or motors 175, 178 are disposed within the housing (not shown) of the forearm 116C.

One implementation of the internal components of the upper arm 116B constructed and arranged to actuate the upper arm roll joint 104 is shown in FIGS. 13 and 14. In this implementation, a motor 142 and gearhead combination controlled by a PCB 112 drives a drive gear 143 and corresponding driven gear 144 where the output/driven gear 144 is supported by a shaft 148 and bearings 150. The output shaft 152 and output spur gear 144 can have a mating feature 146 that mates to the shoulder pitch joint 102 output link 140 (shown in FIG. 10).

One implementation of the internal components of the upper arm 116B configured to operate the elbow joint 106 is shown in FIGS. 15 and 16. In this implementation, a base motor 154 directly drives a gear set that includes three gears 156, 158, 160 (a drive gear 156, a driven gear 158, and a gearhead gear 160). This gear set 156, 158, 160 transfers the axis of rotation from the axis of the motor 154 to the axis of a worm gear 166. Alternatively, the worm gear 166 can be any other known type of gear.

As best shown in FIG. 16, the gearhead gear 160 from this set drives a motor gearhead 162 that drives a shaft 164 that has a worm gear 166 mounted on it. This worm gear 166 then drives a worm wheel 168 (or other form of wheel) that is connected to the Joint 4 output shaft 170. It should also be noted that the upper arm unit (as shown in FIG. 15) shows a curved concave region 172 on the right side. It is understood that this region 172 is configured to allow for a larger motion of Joint 4 so as to allow the forearm to pass through the region 172.

One implementation of the internal components of the forearm 116C configured or otherwise constructed and arranged to operate the tool roll joint 108 is shown in FIGS. 17 and 18. In these implementations, the tool roll joint 108 drives a tool lumen 174 that holds the tool (or end effector). The tool lumen 174 is designed to mesh with the roll features on the end effector to cause the end effector to rotate about its axis, as shown as axis 5 in FIG. 7. In this implementation, a tool roll motor 175 with a gearhead is used to drive a drive gear 176 and thus drive two driven gears 177A, 177B. The second driven gear of this chain 177B is rigidly mounted to the tool lumen 174, so as to rotate the inner surface 174A of the tool lumen, and correspondingly any inserted end effector.

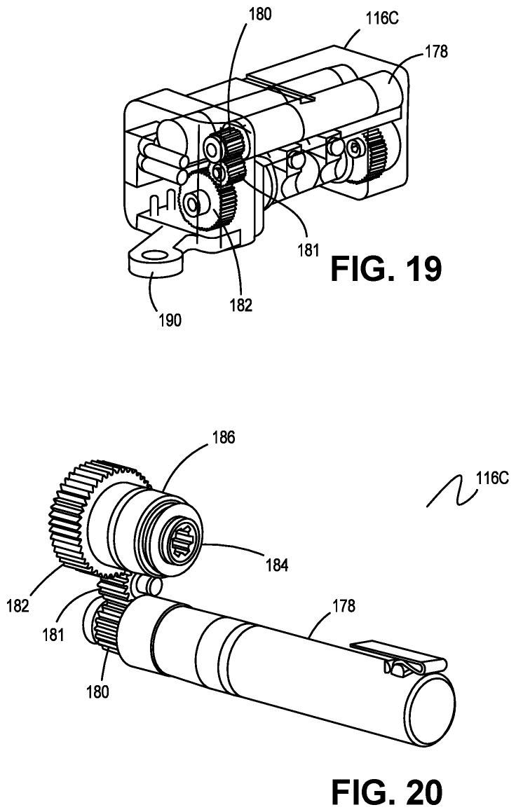

One implementation of a tool actuation joint 109 is shown in FIGS. 19 and 20. In this implementation, the Joint 6 motor 178 does not visibly move the robot. Instead, this tool actuation joint 109 drives a female spline 184 (as best shown in FIG. 20) that interfaces with the end effector and is configured to actuate the end effector to open and close (in those embodiments in which the end effector is a grasper or any other type of end effector that opens and closes). This rotation of the end effector arms such that the end effector opens and closes is also called “tool drive.” The actuation, in one aspect, is created as follows. An actuator 178 is provided that is, in this implementation, a motor assembly 178. The motor assembly 178 is operably coupled to the drive gear 180, which is a spur gear in this embodiment but can be any type of gear. The drive gear 180 is coupled to first 182 and second 183 driven gears such that rotation of the drive gear 180 causes rotation of the two driven gears 182, 183. The driven gears 182, 183 are fixedly coupled to a female tool spline 184, which is supported by bearing pair 186. The female tool spline 184 is configured to interface with a male tool spline feature on the end effector to open/close the tool as directed.

According to one implementation, the end effector can be quickly and easily coupled to and uncoupled from the forearm 116C in the following fashion. With both the roll and drive axes fixed or held in position, the end effector (such as either end effector 56A, 56B) can be rotated, thereby coupling or uncoupling the threads (not shown). That is, if the end effector is rotated in one direction, the end effector is coupled to the forearm 116B, and if it is rotated in the other direction, the end effector is uncoupled from the forearm 116B.

Various implementations of the system 10 are also designed to deliver energy to the end effectors so as to cut and coagulate tissue during surgery. This is sometimes called cautery and can come in many electrical forms as well as thermal energy, ultrasonic energy, and RF energy all of which are intended for the robot.

Alternatively, as mentioned above, certain device embodiments have one or more arms that are actuated by at least one motor coupled to a cable that translates the motive force from the motor to a moveable arm component. Such actuation configurations are also referred to herein as cable-driven actuation or cable actuation and intended to describe any actuation arrangements in which a cable is coupled to both a motor (or other type of actuator) and an actuable component of a robotic device.

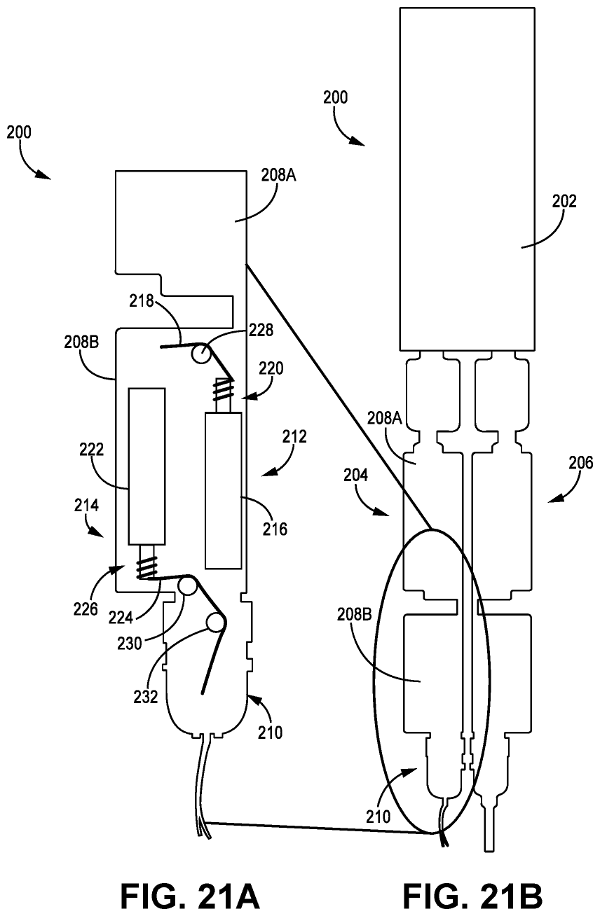

One exemplary implementation of a robotic device 200 with a forearm 208B having two cable-driven actuation assemblies 212, 214 disposed therein or associated therewith is depicted in FIGS. 21A and 21B. More specifically, FIG. 21B shows a robotic device 200 having an elongate body 202 with two robotic arms 204, 206. For purposes of this application, the discussion will focus on the right arm 204, but it is understood that the same or similar cable-driven assemblies can be incorporated into the left arm 206. The right arm 204 has an upper arm 208A, a forearm 208B, and an end effector 210.

FIG. 21B provides an expanded view of the right forearm 208B, which contains the two cable-driven actuation assemblies 212, 214. The first actuation assembly 212 is the tool roll actuation assembly 212 and has a first actuator 216 coupled to a first cable 218 via a first rotating drive mechanism (also referred to herein as a “spool”) 220. The cable 218 extends from the spool 220 to the first target actuable component of the forearm 208B. More specifically, in this example, the cable 218 extends to the tool roll mechanism (not shown) and is operably coupled thereto such that the actuation assembly 212 can be used to actuate the tool roll mechanism.

The second actuation assembly 214 is the tool open/close actuation assembly 214 and has a second actuator 222 coupled to a second cable 224 via a second rotating drive mechanism (also referred to herein as a “spool”) 226. The cable 224 extends from the second spool 226 to the second target actuable component of the forearm 208B. More specifically, in this example, the cable 224 extends to the tool open/close mechanism (not shown) and is operably coupled thereto such that the actuation assembly 214 can be used to actuate the tool open/close mechanism.

In some embodiments, the actuators 216, 222 are motors 216, 222. For example, the motors 216, 222 can be brushless direct current motors with gearheads. Alternatively, the actuators 216, 222 can be any motors as described elsewhere herein or any other known motors for use in such devices. Alternatively, the actuators 216, 222 can be any known actuators.

The cables 218, 224 in this specific implementation and any other embodiments may require one or more pulleys to properly position the cables and/or tensioning mechanisms to ensure proper tension of the cables. For example, a first pulley 228 is disposed within the forearm 208B to route or otherwise control the position of the first cable 218 as shown. Similarly, second and third pulleys 230, 232 are disposed within the forearm 208B to route or otherwise control the position of the second cable 224 as shown. It is understood that the specific number and positioning of any pulleys will depend on the specific arm component and actuation assembly.

According to other embodiments, any such cable-driven actuation assembly can be used to actuate any actuable component. More specifically, the cable of such an assembly is coupled to a component that requires motive force to operate/function. In one specific alternative example, the cables 218, 224 are rotary drive cables. In a further alternative, one of the two cables 218, 224 can be one type of drive cable (such as a push/pull cable, for example) while the other is another type of drive cable (such as a rotary drive cable, for example).

Any spool implementation herein can be sized to allow some length of cable to be wound around the spool. The size of the spool can be determined based on the dimensions of the actuation mechanism.

In this specific embodiment, both of the actuation mechanisms 212, 214 are locally actuated mechanisms 212, 214. That is, the actuators 216, 222 are positioned locally, which means that they are disposed in the same component as the actuable components that are actuated by the actuation mechanisms. In other words, the actuators 216, 222 and the actuable components are both disposed within the forearm 208B. In various alternative implementations as will be described in additional detail below, the actuators 216, 222 are not disposed in the same component as the actuable components. That is, either or both of the actuators 216, 222 can be disposed in the forearm 208B, the upper arm 208A, the elongate body 202, or at some location external to the device 200.

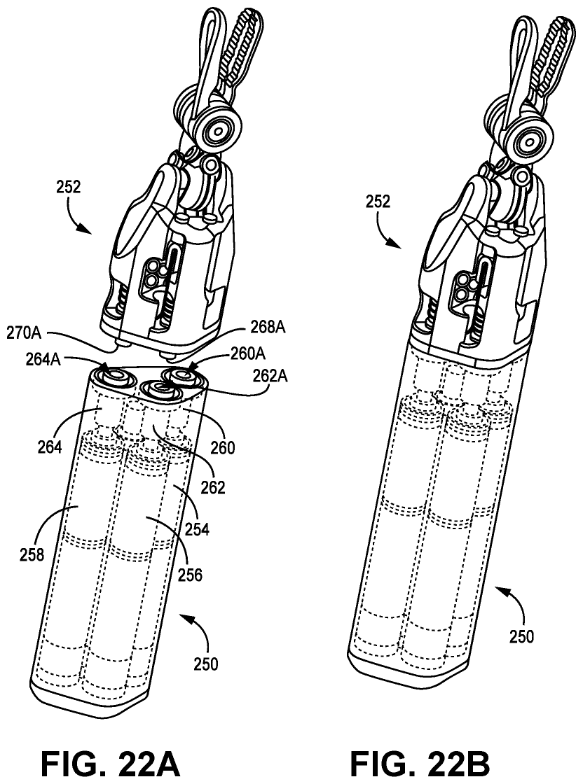

A further, more detailed exemplary embodiment of locally actuated cable-driven actuation assemblies is shown in FIGS. 22A-28B. More specifically, as best shown in FIGS. 22A-22B, a forearm 250 is provided to which a grasper end effector 252 is removably coupleable. The forearm 250 has three actuators 254, 256, 258 disposed therein, with each of the actuators 254, 256, 258 having a rotatable drive component 260, 262, 264, each of which has a mateable female structure 260A, 262A, 264A at its distal end. Each of the mateable female structures 260A, 262A, 264A is mateable with a corresponding mateable male structure such that the motive force can be transferred from each rotatable drive component 260, 262, 264 via the mateable structures, as will be described in additional detail below.

Continuing with FIGS. 22A and 22B, the detachable end effector 252 has three mateable male structures 266A, 268A, 270A (with 266A not being visible in the figures due to the perspective) extending from its proximal end as shown that are mateable with the mateable female structures 260A, 262A, 264A. The three male structures 266A, 268A, 270A are rotatable and configured to mate with the female structures 260A, 262A, 264A. Further, the male structures 266A, 268A, 270A are fixedly coupled to rotatable driven components (also referred to herein as “spools”) 296, 312, 330 disposed within the end effector 252, as discussed in additional detail below. As such, the female (260A, 262A, 264A) and male (266A, 268A, 270A) structures make it possible to transfer motive force from the actuators 254, 256, 258 to the driven spools 296, 312, 330 when the end effector 252 is coupled to the forearm 250.

In one specific implementation, the female (260A, 262A, 264A) and male (266A, 268A, 270A) coupling/motive transfer structures are mateable torque-transferring drive interfaces. Alternatively, any known mateable structures that allow for removable coupling and transfer of rotational motive force can be used.

As will be explained in further detail below, the three actuators 254, 256, 258 provide motive force for articulation of three actuable components motions of the end effector 252. In this embodiment, each of the actuation assemblies (with each assembly being an actuator and the cable coupled thereto) are local (or disposed locally) in that both the actuation assembly and the actuable component coupled thereto are disposed within the same device component (in this case, the forearm 250 with the end effector 252 coupled thereto).

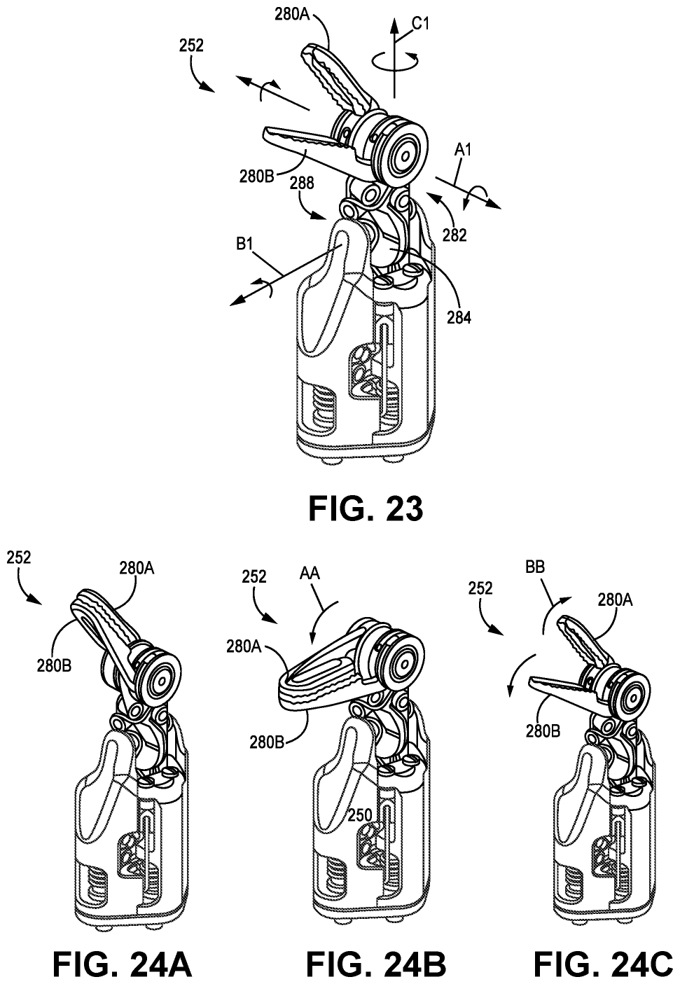

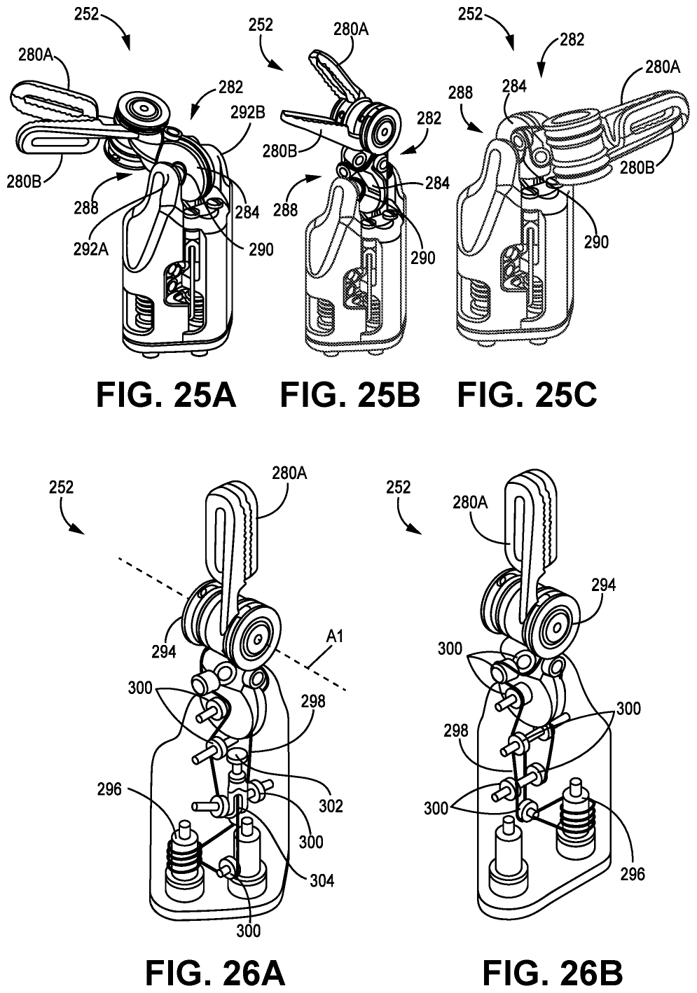

As best shown in FIGS. 23-24C, according to certain embodiments, the end effector 252 is a grasper 252 (as mentioned above), which has two independently-moving paddles 280A, 280B. More specifically, the first paddle 280A and the second paddle 280B both rotate around the same axis A1 to effectuate both an open/close motion as shown in FIG. 24C, and a wrist pitch motion as shown in FIGS. 24A-B. Further, both paddles 280A, 280B (and the entire grasper assembly 282, which is made up of the paddles 280A, 280B and the grasper body 284 as discussed in detail below) can also rotate together around axis B1 to effectuate a wrist yaw motion, as shown in FIGS. 25A-C. It should be noted that the third axis (Cl) depicts the axis around which the entire end effector 252 and forearm 250 can rotate as a result of the forearm being able to rotate around its own axis (“roll”), which is a motion that is not effectuated by the actuators 254, 256, 258 discussed herein in relation to FIGS. 22A-28B.

Focusing on the independent rotation of the paddles 280A, 280B around axis A1, the two different motions can be accomplished in the following fashion. The open/close motion as shown in FIG. 24C can be accomplished by rotating the two paddles 280A, 280B in different directions. More specifically, to open the two paddles 280A, 280B, they are urged to rotate away from each other as represented by the arrows BB in FIG. 24C. In contrast, to close the two paddles 280A, 280B, they are urged to rotate toward and into contact with each other. Further, the wrist-like motion as shown in FIGS. 24A-B can be accomplished by rotating the paddles 280A, 280B in the same direction. For example, to move the two paddles 280A, 280B from the position depicted in FIG. 24A to the position depicted in FIG. 24B, both paddles 280A, 280B are urged to rotate in the same direction as shown by arrow AA.

Turning now to the rotation of the grasper assembly 282, the wrist yaw motion can be accomplished in the following fashion. As shown in FIGS. 25A-C, the grasper assembly 282 includes a grasper body 284 and the two graspers 280A, 280B, which are rotatably coupled to the grasper body 284 around the axis A1 discussed above. The grasper body 284 is rotatably coupled to the end effector body 286 at the wrist joint 288. More specifically, in accordance with one embodiment, the wrist joint 288 includes a pin 290 that extends between the two end effector body protrusions 292A, 292B (as best shown in FIG. 25A) such that the grasper body 284 can be rotatably coupled to the pin 290. As such, the grasper body 284 can rotate around the pin 290 at the axis B1 (which is perpendicular to the axis A1), thereby allowing for the entire grasper assembly 282 to rotate around axis B1, resulting in the wrist yaw motion as depicted in FIGS. 25A-25C.

The combination of actuation assemblies produce the three motions that result in three degrees of freedom, which include the two different wrist motions and the open/close motion. The operation of the actuation assemblies to accomplish each of these three motions will now be explained in detail.

The actuation of paddle 280A rotating around axis A1 is depicted in FIGS. 26A-B according to one embodiment, with FIG. 26A depicting a first perspective view of the end effector 252 (the same perspective view provided in FIGS. 22A-24C) and FIG. 26B depicting a second perspective view that is 180° in relation to the first view (thereby providing a view of an opposite side of the end effector 252 in comparison to FIG. 26A). The paddle 280A is fixedly coupled to a first driven wheel 294 such that rotation of the wheel 294 causes rotation of the paddle 280A around axis A1. Further, FIGS. 26A-B depict the first driven spool (or “mandrel”) 296 coupled to the male mateable structure 270A as discussed above (such that actuator 258 is rotationally coupled to the driven spool 296 when the end effector 252 is coupled to the forearm 250). A first cable 298 forms a closed loop such that the cable 298 is coupled to the first driven mandrel 296 and further ultimately extends to and is coupled with the first driven wheel 294. More specifically, in this particular implementation, the cable 298 is routed through (or otherwise positioned within) the end effector 252 via a set of pulleys 300 that ultimately result in the cable 298 extending from the driven mandrel 296 to the driven wheel 294 and back to the mandrel 296 such that rotation of the mandrel 296 causes translation of the cable 298, which causes rotation of the wheel 294 (which is coupled to the first paddle 280A). It is understood that any number of pulleys 300 that are positioned in any configuration can be provided to ensure proper positioning of the cable 298 and eliminate any unwanted slack therein. Further, as best shown in FIG. 26A, a tensioning screw 302 coupled to a tensioning pulley 304 is provided to adjust the cable 298 to the desired tension. Thus, actuation of the actuator 258 can cause rotation of the first paddle 280A around the axis A1.

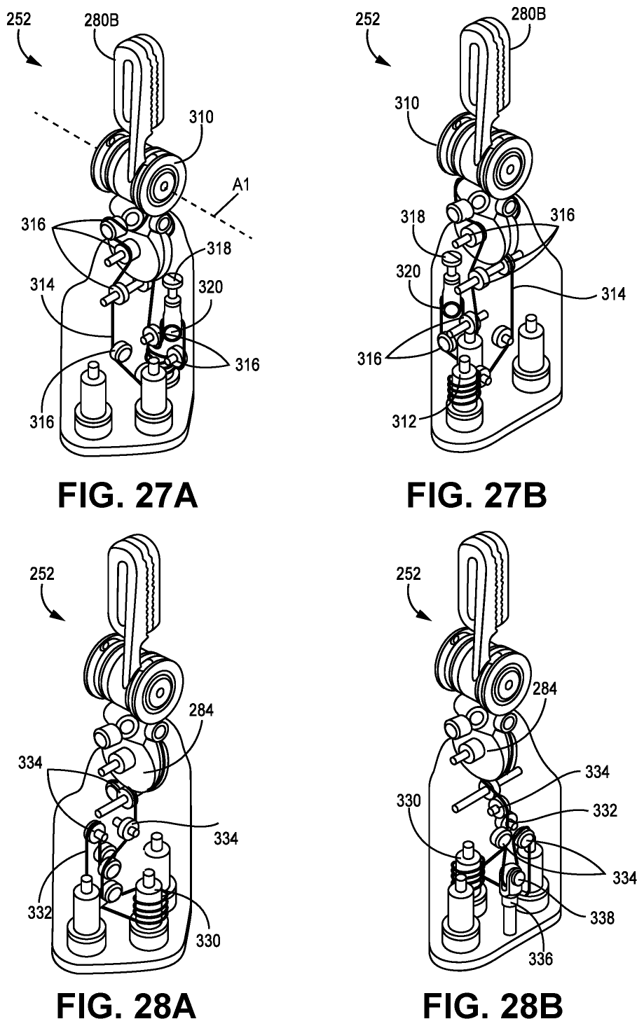

The actuation of paddle 280B rotating around axis A1 is depicted in FIGS. 27A-B according to one embodiment, with FIG. 27A depicting a first perspective view (the same perspective view provided in FIG. 26A) and FIG. 27B depicting a second perspective view (the same view provided in FIG. 26B). The paddle 280B is fixedly coupled to a second driven wheel 310 such that rotation of the wheel 310 causes rotation of the paddle 280B around axis A1. Further, FIG. 27B depicts the second driven spool (or “mandrel”) 312 coupled to the male mateable structure 266A as discussed above (such that actuator 254 is rotationally coupled to the driven spool 312 when the end effector 252 is coupled to the forearm 250). A second cable 314 forms a closed loop such that the cable 314 is coupled to the second driven mandrel 312 and further ultimately extends to and is coupled with the second driven wheel 310. More specifically, in this particular implementation, the second cable 314 is routed through (or otherwise positioned within) the end effector 252 via a set of pulleys 316 that ultimately result in the cable 314 extending from the driven mandrel 312 to the driven wheel 310 and back to the mandrel 312 such that rotation of the mandrel 312 causes translation of the cable 314, which causes rotation of the wheel 310 (which is coupled to the second paddle 280B). It is understood that any number of pulleys 316 that are positioned in any configuration can be provided to ensure proper positioning of the cable 314 and eliminate any unwanted slack therein. Further, a tensioning screw 318 coupled to a tensioning pulley 320 is provided to adjust the cable 314 to the desired tension. Thus, actuation of the actuator 254 can cause rotation of the second paddle 280B around the axis A1.

The actuation of the grasper assembly 282 rotating around axis B1 is depicted in FIGS. 28A-B according to one embodiment, with FIG. 28A depicting a first perspective view (the same perspective view provided in FIGS. 26A and 27A) and FIG. 28B depicting a second perspective view (the same view provided in FIGS. 26B and 27B). As discussed above, the grasper assembly 282 has a grasper body 284 with the paddles 280A, 280B such that rotation of the body 284 at wrist joint 288 causes rotation of the assembly 282 around axis B1. Further, FIGS. 28A-B depict the third driven spool (or “mandrel”) 330 coupled to the male mateable structure 268A as discussed above (such that actuator 256 is rotationally coupled to the driven spool 330 when the end effector 252 is coupled to the forearm 250). A third cable 332 forms a closed loop such that the cable 332 is coupled to the third driven mandrel 330 and further ultimately extends to and is coupled with the grasper body 284. More specifically, in this particular implementation, the third cable 332 is routed through (or otherwise positioned within) the end effector 252 via a set of pulleys 334 that ultimately result in the cable 332 extending from the driven mandrel 330 to the grasper body 284 and back to the mandrel 330 such that rotation of the mandrel 330 causes translation of the cable 332, which causes rotation of the grasper body 284 and thus the grasper assembly 282. It is understood that any number of pulleys 334 that are positioned in any configuration can be provided to ensure proper positioning of the cable 332 and eliminate any unwanted slack therein. Further, a tensioning screw 336 coupled to a tensioning pulley 338 is provided to adjust the cable 332 to the desired tension. Thus, actuation of the actuator 256 can cause rotation of the grasper assembly 282 around the axis B1, which results in wrist yaw motion.

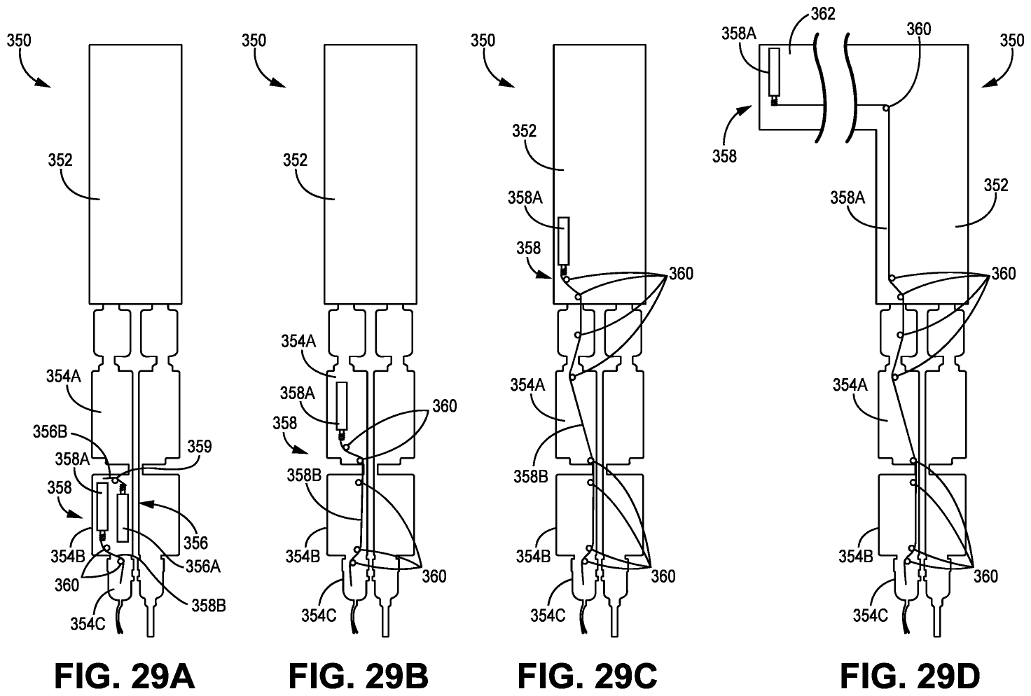

As mentioned above, the various actuator/cable actuation assemblies disclosed or contemplated according to any of the embodiments herein allow for the positioning of the actuator in any number of different locations within or external to the robotic device. In one specific example, FIGS. 29A-D depict various different implementations in which the actuator of an actuator/cable actuation assembly can be located in a variety of locations while providing actuation of the same actuable component (which in this case is the open/close action of the end effector). Alternatively, the actuable component can be any such actuable component within a robotic device embodiment.

In one specific exemplary embodiment as shown in FIG. 29A (which is similar to the implementation depicted in FIGS. 21A-B and discussed above), a robotic device 350 has a device body 352 and at least one arm (in this case, a right arm) 354 with an upper arm 354A, a forearm 354B, and an end effector 354C attached to the forearm 354B. Two actuation assemblies 356, 358 are disposed within or otherwise associated with the forearm 354B. More specifically, the first actuation assembly (the tool roll actuation assembly) 356 is an actuator/cable assembly 356 having an actuator 356A and an attached cable 356B that is coupled to the actuable tool roll mechanism (not shown) and positioned between the actuator 356A and tool roll mechanism via one or more pulleys 359. As such, actuation of the assembly 356 causes actuation of the tool roll mechanism, thereby causing the end effector 354C to rotate around its axis. In addition, the second actuation assembly (the open/close actuation assembly) 358 is an actuator/cable assembly 358 having an actuator 358A and an attached cable 358B that is coupled to the actuable end effector 354C and positioned between the actuator 358A and the end effector 354C via one or more pulleys 360. As such, actuation of the assembly 358 causes actuation of the end effector 354C to open and close. In this specific implementation, the actuators 356A, 358A of both assemblies are disposed within or associated with the forearm 354B such that the actuators 356A, 358A are disposed “locally” in relation to the actuable components to which they are coupled. The specific number and positioning of the pulleys (such as pulleys 359, 360) in this embodiment and the additional embodiments disclosed or contemplated below and elsewhere in this application can vary as needed depending on the various parameters relating to the arm, the actuation assembly, and other known variables within any known device in which such an actuation assembly may be incorporated. Further, it is understood that other mechanisms and/or structures can be used in addition to or in place of the pulleys to position the cable (such as cables 356B, 358B) as desired/needed.

Alternatively, as shown in FIG. 29B, either or both of the actuation assemblies discussed above can be configured such that the actuator is disposed within or otherwise associated with the upper arm 354A (instead of the forearm 354B). More specifically, in this exemplary embodiment, the open/close actuation assembly 358 is configured such that the actuator 258A is disposed within or otherwise associated with the upper arm 354A, with the cable 358B extending from the actuator 258A in the upper arm 354A through the forearm 354B to the end effector 354C attached to the forearm 354B. More specifically, the cable 358B is positioned within the upper arm 354A and the forearm 354B as desired via appropriately positioned pulleys 360. The specific number and positioning of the pulleys 360 and/or other mechanisms can vary as mentioned above. In this embodiment, while not shown in FIG. 29B, the tool roll actuation assembly 356 is disposed within the forearm 354B as described above with respect to FIG. 29A.

Alternatively, as shown in FIG. 29C, either or both of the actuation assemblies discussed above can be configured such that the actuator is disposed within or otherwise associated with the device body 352 (instead of the upper arm 354A or the forearm 354B). More specifically, in this exemplary embodiment, the open/close actuation assembly 358 is configured such that the actuator 358A is disposed within or otherwise associated with the device body 352 as shown, with the cable 358B extending from the actuator 358A in the device body 352 through the upper arm 354A and the forearm 354B to the end effector 354C attached to the forearm 354B. More specifically, the cable 358B is positioned within the device body, upper arm 354A, and forearm 354B as desired via appropriately positioned pulleys 360. The specific number and positioning of the pulleys 360 and/or other mechanisms can vary as mentioned above. In this embodiment, while not shown in FIG. 29C, the tool roll actuation assembly 356 is disposed within the forearm 354B as described above with respect to FIG. 29A.

In a further alternative as shown in FIG. 29D, either or both of the actuation assemblies discussed above can be configured such that the actuator is disposed at a location external to the device body 352. More specifically, the actuator 358A can be located in an external controller, a separate actuation component (including, for example, a detachable actuation component that can be removably attached to the device body 352), or any other location from which the cable 358B coupled thereto can extend into the device 350. In this exemplary embodiment as shown, the open/close actuation assembly 358 is configured such that the actuator 358A is disposed in an external controller 362, with the cable 358B extending from the actuator 358A to the device body 352 and through the device body 352, the upper arm 354A, and the forearm 354B to the end effector 354C attached to the forearm 354B. More specifically, the cable 358B is positioned external to the device 350 and through the device body 352, upper arm 354A, and forearm 354B as desired via appropriately positioned pulleys 360. The specific number and positioning of the pulleys 360 and/or other mechanisms can vary as mentioned above. In this embodiment, while not shown in FIG. 29D, the tool roll actuation assembly 356 is disposed within the forearm 354B as described above with respect to FIG. 29A.

In some embodiments, the actuators 356A, 358A in FIGS. 29A-29D are motors. For example, the motors can be brushless direct current motors with gearheads. Alternatively, the actuators 356A, 358A can be any motors as described elsewhere herein or any other known motors for use in such devices. Alternatively, the actuators 356A, 358A can be any known actuators.

While each of the implementations in FIGS. 29A-29D as discussed above are described as having known push/pull cables or known pull and opposing cables, according to other embodiments, the cables 356B, 358B can be rotary drive cables. In a further alternative, one of the two cables 356B, 358B can be one type of drive cable (such as a push/pull cable or opposing pull cables, for example) while the other is another type of drive cable (such as a rotary drive cable, for example). Thus, in the various embodiments utilizing at least one rotary drive cable, no pulleys are required for that cable and thus do not need to be included in the device.

The specific embodiments discussed above and depicted in FIGS. 29A-D are provided as non-limiting examples. Any actuable component can be actuated by an actuator disposed in or associated with any component of a device (or external to such device) through the routing of cables through various lumens and/or pulleys. That is, according to other embodiments, any such cable-driven actuation assembly with an actuator disposed at any of the locations described above can be used to actuate any actuable component. More specifically, the cable of such an assembly is coupled to a component that requires motive force to operate/function. In one specific implementation, all of the actuators of all the actuation assemblies can be disposed at an external location in a fashion similar to that shown in FIG. 29D.

As discussed above, the various actuator/cable actuation assemblies disclosed or contemplated according to any of the embodiments herein allow for combinations of different actuator assemblies within the same device and/or in the same component of the same device. That is, a robotic arm or one segment of such an arm (such as the upper arm or forearm) can contain both an actuator/gear actuation assembly and an actuator/cable assembly. Further, any device can contain any combination of such assemblies.

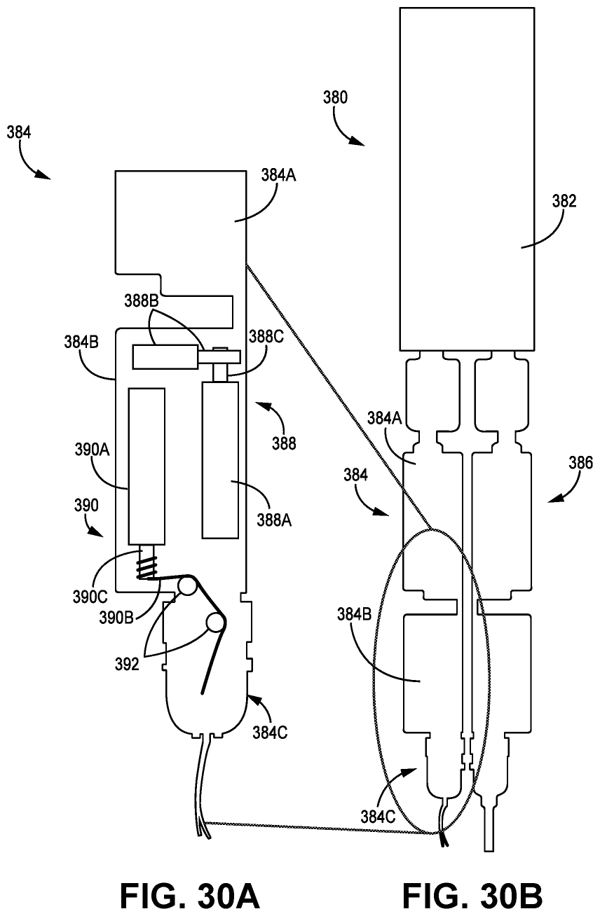

One exemplary implementation of a robotic device 380 with a forearm 384B having two different actuation assemblies 388, 390 disposed therein or associated therewith is depicted in FIGS. 30A and 30B. More specifically, FIG. 30B shows a robotic device 380 having an elongate body 382 with two robotic arms 384, 386. For purposes of this application, the discussion will focus on the right arm 384, but it is understood that the same or similar actuation assemblies can be incorporated into the left arm 386. The right arm 384 has an upper arm 384A, a forearm 384B, and an end effector 384C.

FIG. 30A provides an expanded view of the right forearm 384B, which contains the two actuation assemblies 388, 390. The first actuation assembly 388 is the tool roll actuation assembly 388 and has a first actuator 388A coupled to a driven gear set 388B via a first rotating drive mechanism 388C. The driven gear set 388B is coupled to the first target actuable component of the forearm 384B. More specifically, in this example, the drive gear set 388B is coupled to the tool roll mechanism (not shown). Alternatively, the tool roll actuation assembly 388 can be any known gear driven actuation assembly having any configuration of an actuator and at least one gear.

The second actuation assembly 390 is the tool open/close actuation assembly 390 and has a second actuator 390A coupled to a cable 390B via a second rotating drive mechanism 390C which is, in some embodiments, a spool 390C. The cable 390B extends from the spool 390C to the second target actuable component of the forearm 384B via two appropriately positioned pulleys 392. More specifically, in this example, the cable 390B extends to and is coupled to the tool open/close mechanism (not shown). The specific number and positioning of the pulleys 392 and/or other mechanisms can vary as mentioned above with respect to other embodiments. Alternatively, instead of the cable 290B being a lateral movement cable (such as a push/pull cable or known pull and opposing cable), the cable 290B can be a rotary drive cable. In such embodiments, no pulleys are required for the cable 290B and thus are not included in the device.

Thus, the forearm 384B in this specific implementation has a first actuation assembly 388 that is an actuator/gear assembly 388 and a second actuation assembly 390 that is an actuator/cable assembly 390. Each actuable component in a robotic device has specific torque and speed requirements, and the appropriate actuation assembly that satisfies those requirements can be used for each.

In some embodiments, the actuators 388A, 390A are motors. For example, the motors 388A, 390A can be brushless direct current motors with gearheads. Alternatively, the actuators 388A, 390A can be any motors as described elsewhere herein or any other known motors for use in such devices. Alternatively, the actuators 388A, 390A can be any known actuators.

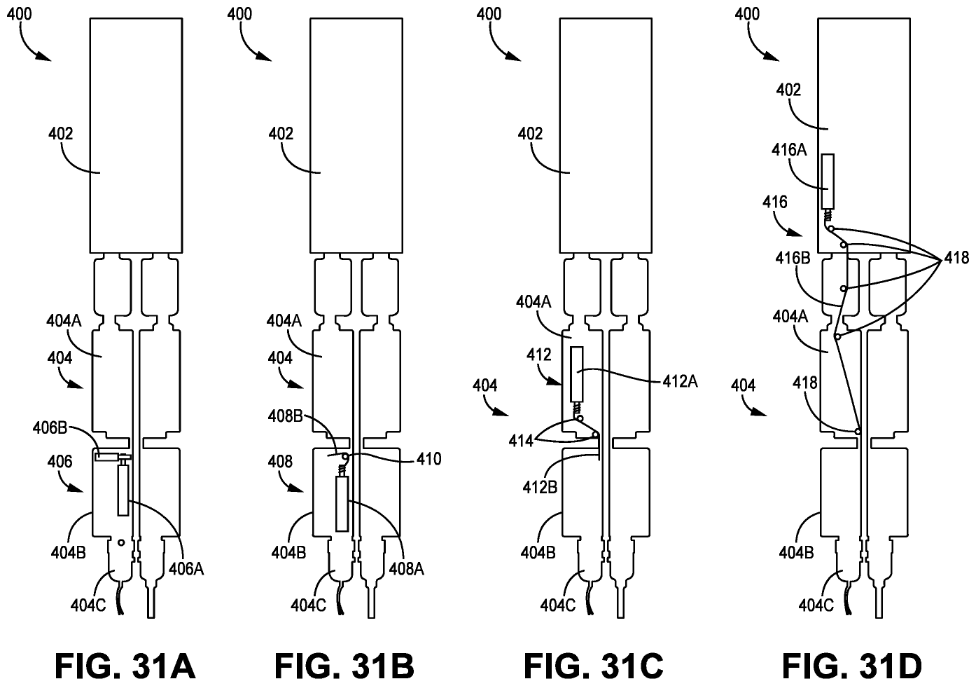

According to other embodiments, any such cable-driven actuation assembly can be used to actuate any actuable component in any configuration disclosed or contemplated in the various embodiments herein, including in combination with one or more actuator/gear assembly. For example, various alternative configurations of assembly combinations are shown in FIGS. 31A-D, in which both the types of actuation assemblies can be combined in the same device and the location of the actuators can vary as well. For purposes of this application, the various device embodiments having at least one cable-driven actuation assembly and at least one gear-driven actuation assembly can be referred to as hybrid devices.

In one specific exemplary embodiment as shown in FIG. 31A, a robotic device 400 has a device body 402 and at least one arm (in this case, a right arm) 404 with an upper arm 404A, a forearm 404B, and an end effector 404C attached to the forearm 404B. In this implementation of a hybrid device 400, one of the actuation assemblies 406 disposed within or otherwise associated with the forearm 404B is an actuator/gear assembly for actuation of the tool roll mechanism, while at least one of the other actuation assemblies (not shown) within the device 400 is a actuator/cable assembly (not shown) according to any of the various embodiments herein. More specifically, the actuation assembly (the tool roll actuation assembly) 406 is an actuator/gear assembly 406 having an actuator 406A and an attached gear set 406B that is coupled to the actuable tool roll mechanism (not shown). As such, actuation of the assembly 406 causes actuation of the tool roll mechanism, thereby causing the end effector 404C to rotate around its axis. Further, in this embodiment, the tool open/close actuation assembly (not shown) is an actuator/cable assembly in a fashion similar to the assembly 390 depicted in FIG. 30A and described above or any other actuator/cable assembly as described elsewhere herein. In addition, according to various embodiments, any other actuation assemblies within this device can be any combination of actuator/cable assemblies and/or actuator/gear assemblies.

Alternatively, as shown in FIG. 31B, the tool roll actuation assembly 408 within the forearm 404B is an actuator/cable assembly 408. More specifically, the actuation assembly 408 has an actuator 408A and an attached cable 408B that is coupled to the actuable tool roll mechanism (not shown) such that the cable 408B extends from the actuator 408A to the mechanism and is positioned via a pulley 410. Alternatively, instead of the cable 408B being a lateral movement cable (such as a push/pull cable or known pull and opposing cable), the cable 408B can be a rotary drive cable. In such embodiments, no pulleys are required for the cable 408B and thus are not included in the device. Further, in this embodiment, the tool open/close actuation assembly (not shown) can be an actuator/gear assembly, or, alternatively, an actuator/cable assembly in a fashion similar to the assembly 390 depicted in FIG. 30A and described above. According to various embodiments, any other actuation assemblies within this device can be any combination of actuator/cable assemblies and/or actuator/gear assemblies.

Further, the various embodiments in which a device has a combination of both at least one actuator/gear assembly and at least one actuator/cable assembly (a hybrid device) can also include at least one actuator that is not disposed in the same component as the actuable component. For example, in FIG. 310 according to one exemplary implementation, the tool roll actuation assembly 412 is configured such that the actuator 412A is disposed within or otherwise associated with the upper arm 404A, with the cable 412B extending from the actuator 412A in the upper arm 404A into the forearm 404B to the tool roll mechanism (not shown) within the forearm 404B. More specifically, the cable 412B is positioned within the upper arm 404A and the forearm 404B as desired via appropriately positioned pulleys 414. The specific number and positioning of the pulleys 414 and/or other mechanisms can vary as mentioned above. Alternatively, instead of the cable 412B being a lateral movement cable (such as a push/pull cable or known pull and opposing cable), the cable 412B can be a rotary drive cable. In such embodiments, no pulleys are required for the cable 412B and thus are not included in the device. Further, in this embodiment, the tool open/close actuation assembly (not shown) can be an actuator/gear assembly, or, alternatively, an actuator/cable assembly in a fashion similar to the assembly 390 depicted in FIG. 30A and described above. Like the tool roll actuation assembly, the actuator in the tool open/close actuation assembly can have an actuator that is disposed within the forearm 404B, the upper arm 404A, or elsewhere in the device 400. According to various embodiments, any other actuation assemblies within this device can be any combination of actuator/cable assemblies and/or actuator/gear assemblies, with the actuators disposed locally or at least one component away from the actuable component (or external to the device) as described in various embodiments herein.

In another exemplary embodiment as shown in FIG. 31D, the tool roll actuation assembly 416 is configured such that the actuator 416A is disposed within or otherwise associated with the device body 402, with the cable 416B extending from the actuator 416A in the device body 402 through the upper arm 404A and into the forearm 404B to the tool roll mechanism (not shown) within the forearm 404B. More specifically, the cable 416B is positioned within the device body 402, the upper arm 404A, and the forearm 404B as desired via appropriately positioned pulleys 418. The specific number and positioning of the pulleys 418 and/or other mechanisms can vary as mentioned above. Alternatively, instead of the cable 416B being a lateral movement cable (such as a push/pull cable or known pull and opposing cable), the cable 416B can be a rotary drive cable. In such embodiments, no pulleys are required for the cable 416B and thus are not included in the device. Further, in this embodiment, the tool open/close actuation assembly (not shown) can be an actuator/gear assembly, or, alternatively, an actuator/cable assembly in a fashion similar to the assembly 390 depicted in FIG. 30A and described above. Like the tool roll actuation assembly, the actuator in the tool open/close actuation assembly can have an actuator that is disposed within the forearm 404B, the upper arm 404A, the device body 402, or elsewhere in the device 400. In accordance with other alternative embodiments, the actuators for either or both of the tool roll actuation assembly and/or the tool open/close actuation assembly can be disposed at some external location as described elsewhere herein. According to various embodiments, any other actuation assemblies within this device can be any combination of actuator/cable assemblies and/or actuator/gear assemblies, with the actuators disposed locally or at least one component away from the actuable component (or external to the device) as described in various embodiments herein.

According to some exemplary implementations having a high power tool open/close mechanism (which may require more force than other such mechanisms), a local actuator/gear assembly is coupled to the open/close mechanism while an actuator/cable assembly is provided for tool roll with the actuator being disposed more “remotely” (disposed within the upper arm, the device body, or external to the device). Alternatively, the opposite can be true, such that a local actuator/cable assembly is coupled to the open/close mechanism while the tool roll actuation assembly can be either cable-driven or gear-driven and can have an actuator that is local or remote. In accordance with another embodiment, both the tool roll and tool open/close mechanisms in the forearm can be coupled to actuation assemblies with actuators disposed within the device body and the remaining actuation assemblies are disposed locally. All combinations of actuation assemblies are contemplated herein.

In accordance with other embodiments, any of the actuable components within any robotic device disclosed or contemplated herein can be actuated with an assembly having an actuator that is disposed at least one component away from the actuable component. More specifically, any of the rotatable joints within any such device can be actuated by an actuator disposed anywhere in the device or external to the device.

Further, any type of actuable component is contemplated for any embodiment herein as well. That is, the various actuable components that are contemplated herein—in addition to the specific grasper end effector and other end effectors and actuable components described herein—include any end effector or other actuable component that can be incorporated into a robotic device. For example, in some other exemplary embodiments, another actuable component that can be actuated by any of the assemblies disclosed or contemplated herein is an end effector with a linear drive for a cutting blade. Any of the actuation assemblies in any of the embodiments herein can be used to actuate such a linear drive, including a remote actuator with a linear push/pull cable.

As noted elsewhere, any of the actuators disclosed or contemplated herein can be any known type of actuator, including, but not limited to, motors, muscle wire, hydraulics, pneumatics, etc.

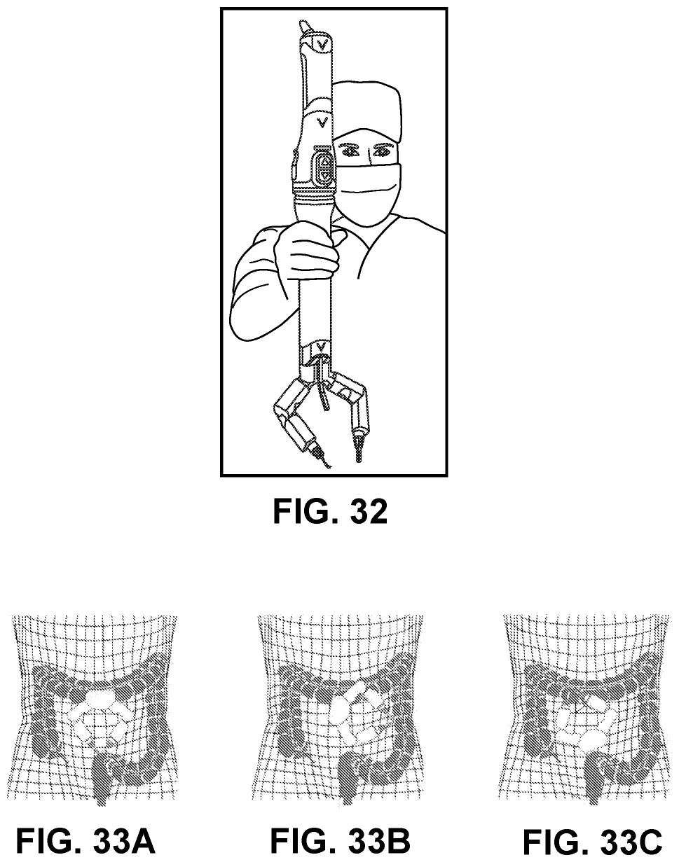

In accordance with various device embodiments herein, the various actuation assembly configurations herein make it possible for the devices to be handheld (as shown in FIG. 32) or of similar size, which provides various advantages during surgical procedures. That is, the ability to position one or more actuators at a location that is at least one component away from the actuable component allows for the components to have smaller dimensions because such components do not have to contain actuators. For example, if all of the actuators for the entire device are disposed within the device body, the arms can have smaller dimensions in comparison to arms containing actuators. In another example, if all of the actuators for the entire device are disposed external to the device, the arms and the device body can have smaller dimensions in comparison to similar arms and a device body containing actuators. More specifically, the various devices herein (including the device body, camera, and any arms attached thereto) can have a weight ranging from about 2 pounds to about 25 pounds. Alternatively, the weight can be any weight that is about 25 pounds or less, about 10 pounds or less, about 5 pounds or less, or about 2 pounds. Thus, the various device implementations herein are small enough that they can be easily stored, transported, and set up for use, along with being easily deployed and repositioned during use. A further advantage of the device size is that the device saves space in the operating room, including, for example, the space above the patient and next to the operating table.

Yet another advantage of the device implementations herein and specifically the size thereof is that the various devices herein are not so large that they are required to be attached to or resting on the ground or to the wall or ceiling (which is a common requirement of other such devices/systems as a result of their size). Instead, the device embodiments herein are sized such that each can be attached to and supported by the standard rail on the side of the operating table (such as the setup of FIG. 1 above with the support arm attached to the operating table) or a similar attachment mechanism or method.

In addition, the size of the device embodiments herein allow for such a device to be easily repositioned to different locations/positions within the target cavity of the patient, such as the peritoneal cavity. For example, as shown in FIGS. 33A-C, the distal end of the device (the portion of the device—including the distal end of the body and the arms—disposed within the patient cavity) can be easily positioned near the rectum (FIG. 33A), near the colon (FIG. 33B), and near the transverse colon (FIG. 33C). The repositioning of the device to move it into any of these three positions is simple and easy as a result of the size of the device. For example, in certain embodiments, the mounting structure (such as a support arm) need not be moved from one side of the operating table to the other as a result of the small size of the device implementations herein, and instead the device can simply be repositioned in relation to the support arm.

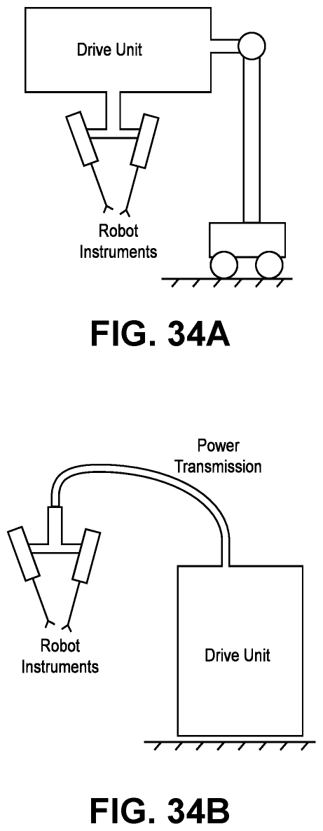

In contrast, various prior art systems require larger components that are cumbersome and restrict the use of such systems in comparison to the various embodiments herein. For example, various known systems require that the actuators be disposed within an external proximal component such as a drive unit that is connected to the device via a direct connection or a power transmission mechanism or the like. Due to the size of the systems, as shown in FIG. 34A, such drive units typically need to be supported with an external attachment such as an extended arm attached to a cart, a wall or ceiling attachment, or the like. Other such systems have drive units that need to be supported with a floor base unit as shown in FIG. 34B. In contrast, the various device and system embodiments herein require no such carts, wall/ceiling attachments, or floor units to support such drive units.

In accordance with one exemplary embodiment, a robotic device 500 having an external actuation unit 512 with multiple external actuators is depicted in FIGS. 35A-35E. That is, as will be discussed in further detail below, the device 500 has multiple separate actuation assemblies, each having an external actuator (in the external actuation unit 512) with a force transmission cable attached thereto that extends into the device 500 and is coupled to the intended actuable component of the device 500. As such, force generated by each actuator in the actuator unit 512 is transmitted to the intended actuable component via the force transmission cable coupled thereto, as will be described in additional detail below. The device 500 can be incorporated into the exemplary system 10 discussed above or any other system disclosed or contemplated herein.

The device 500 has an elongate body 502 having a distal section 502A and proximal section 502B, two anthropometric robotic arms 504, 506 (a right arm 504 and a left arm 506) operably coupled to a distal end of the distal section 502A of the body 502, and an imaging device (or “camera”) 508 removably disposed through the elongate body 502 such that the distal end of the camera 508 is disposed between the two arms 504, 506 as shown. The handle 510 at the proximal end of the camera 508 is coupled to a proximal end of the proximal section 502B, as will be discussed in further detail below. Except as expressly discussed below, the various components and features of the device 500 can be substantially similar to any of the embodiments disclosed or contemplated herein, and further can be substantially similar to any of the components and/or features of the devices disclosed in U.S. application Ser. Nos. 16/736,329, 16/926,025, 17/075,122, and 17/367,915, all of which are incorporated herein by reference in their entireties.

Further, an attachment or stabilization device 516 is also provided that can be removably coupled to the elongate device body 502 to maintain the desired position of the device 500 in relation to the surgical space and the patient (not shown). Any known attachment device can be used.

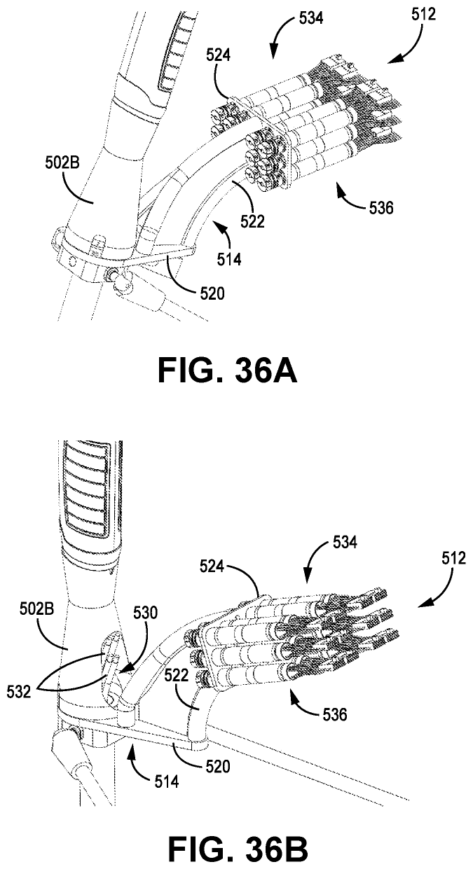

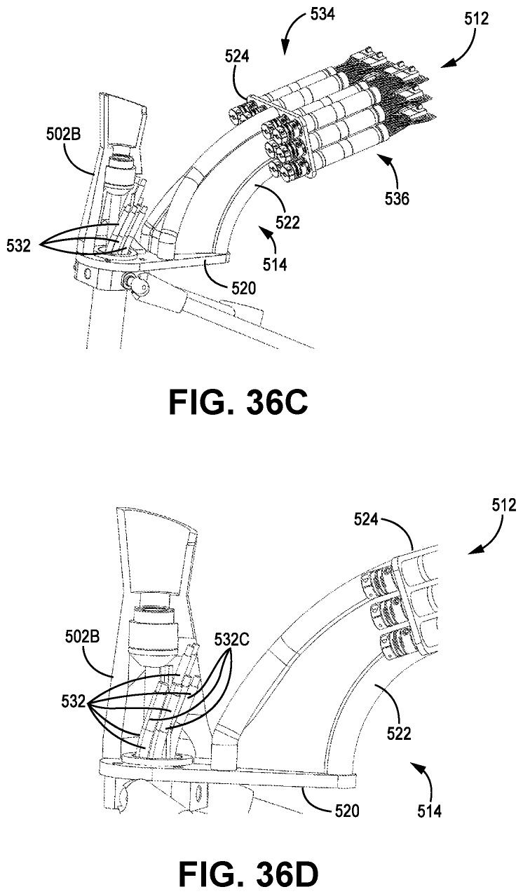

In this exemplary implementation as shown in FIGS. 36A-36D, the actuation unit 512 is coupled to the elongate body 502 via an arm 514 that extends from the elongate body 502 to the actuation unit 512 as shown. The arm 514 in this embodiment has a radial link 520 attached to and extending radially from the body 502, and an extension link 522 attached to the radial link 520, with the actuation unit 512 attached to the extension link 522. More specifically, the extension link 522 in this exemplary embodiment is a curved extension link 522 that extends both radially and axially (in relation to the elongate body 502) from the radial link 520 as shown. Alternatively, the arm 514 can be any structure that can couple the unit 512 to the body 502 and position the unit 512 in relation to the body 502 as desired.

In one embodiment, the arm 514 (and more specifically, the extension link 522) is coupled to the actuation unit 512 via a support plate 524. That is, the actuation unit 512 has a support plate 524 that couples to the arm 514 and has all of the actuators 534, 536 coupled thereto as shown. More specifically, each of the actuators 534, 536 is attached to the support plate 524 such that the actuators 534, 536 are disposed on one side of the plate 524 and the actuator gears extend through openings (not shown) in the plate 524 to the other side of the plate 524.

In some embodiments, the actuators 534, 536 are motors 534, 536. For example, the motors 534, 536 can be brushless direct current motors with gearheads from Maxon. Alternatively, the actuators 534, 536 can be any motors as described elsewhere herein or any other known motors for use in such devices. Alternatively, the actuators 534, 536 can be any known actuators.

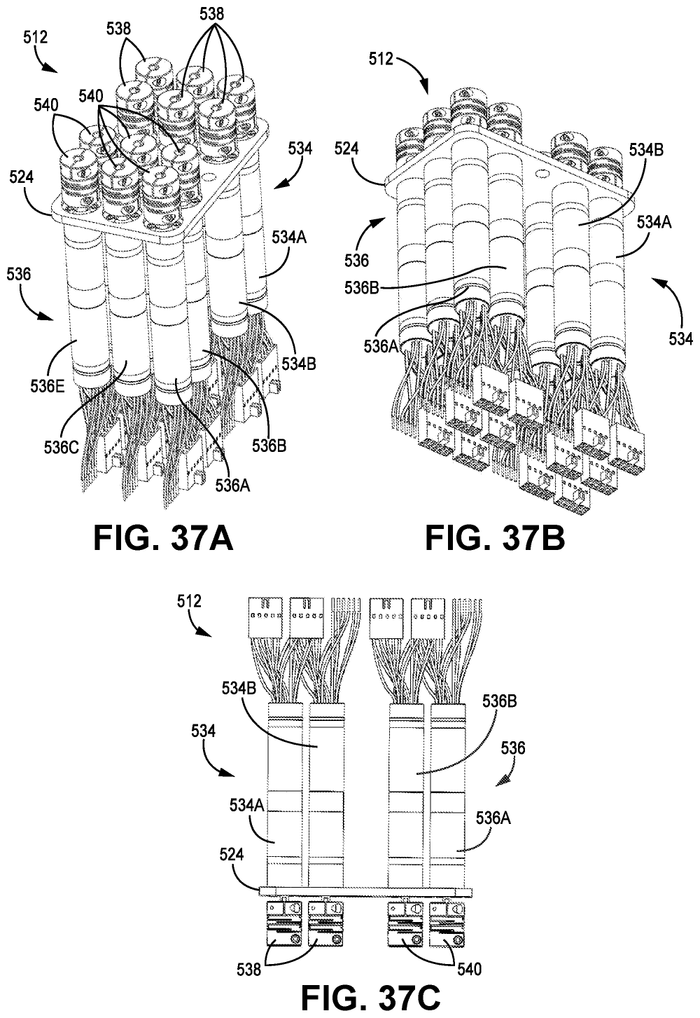

As best shown in FIGS. 37A-37C, the actuation unit 512 has twelve motors 534A-F, 536A-F as shown. More specifically, the unit 512 has six right arm actuators 534 and six left arm actuators 536. Thus, the unit 512 has the same number of right arm actuators 534 as the number of degrees of freedom in the right arm 504 (which is six in this case), and similarly has the same number of left arm actuators 536 as the number of degrees of freedom in the left arm 506 (which is also six in this case). Alternatively, the actuation unit 512 has any number of actuators to match the number of degrees of freedom in the device 500.

According to one implementation, the actuation unit 512 is easily detachable from the elongate body 502 and/or from the arm 514. For example, in those embodiments in which the device 500 is disposable, the unit 512 can be easily attachable and removable such that the unit 512 can be attached to the device 500 prior to use and further can be easily detached after use such that the unit 512 can be retained while the device 500 is disposed of.

Returning to FIGS. 36B-D, the motors 534, 536 are coupled to the actuable components of the device 500 via the motive force transfer cables 532. More specifically, each actuation assembly (described in further detail below) is made up of one of the motors 534, 536 and the cable 532 coupled thereto. While not shown in the figures, each cable 532 is operably coupled at its proximal end to a separate one of the motors 534, 536 and extends from the actuation unit 512 through an opening 530 in the proximal section 502B and into the elongate body 502 as shown.

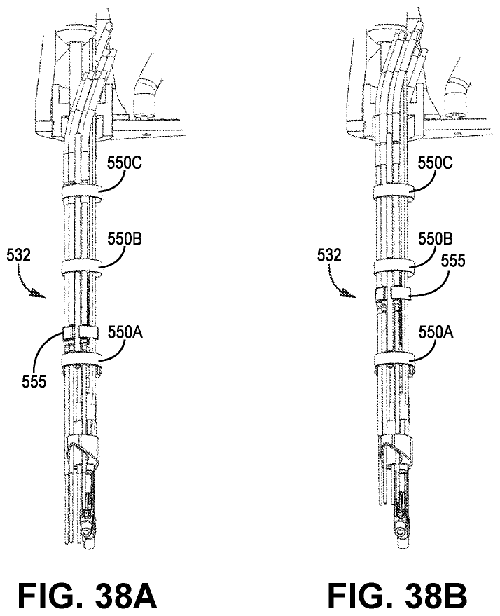

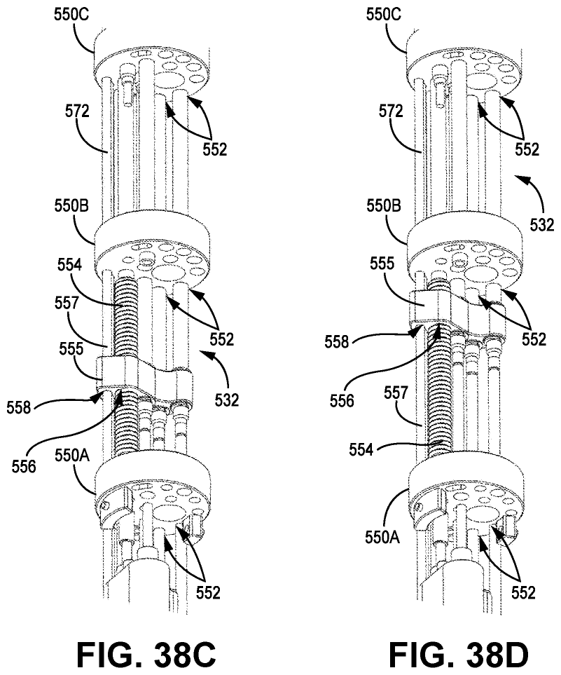

As shown in FIGS. 38A-D, according to one embodiment, each cable 532 extends distally along the interior of the elongate body 502. More specifically, the elongate body 502 has three interior body supports 550A, 550B, 550C, each of which is disposed in the interior of the elongate body 502 at a different location along the length thereof as shown. Further, each body support 550A, 550B, 550C has openings 552 defined therein to allow each of the cables 532 to pass therethrough such that each separate cable 532 passes through a separate opening 552 as shown. As such, the supports 550A, 550B, 550C provide structural support to the elongate body 502 and the cables 532 disposed therethrough.

According to certain implementations, each of the force transmission cables 532 is a flexible rotary torque transmission cable 532. More specifically, any known flexible rotary torque transmission cable 532 can be used. As such, each cable 532 has an outer sheath or casing with a rotatable shaft disposed within the outer sheath such that the rotatable shaft transmits the motive force from the specific actuator to which the cable 532 is coupled at its proximal end to the intended actuable component to which the cable 532 is coupled at its distal end. In one specific implementation, the rotary torque transmission cable 532 and any other rotary transmission cable disclosed or contemplated herein is a flexible rotary shaft, which is commercially available from Suhner Manufacturing Co. In some embodiments, certain of the flexible rotary shafts are custom-made flexible cables with an internal rotary shaft that is rotatable within an exterior sheath. Certain specific implementations include flexible rotary cables with an internal rotary shaft made up of multiple layers of small diameter counter wound wires (thereby allowing for bi-directional rotation without unwinding the rotary shaft). Alternatively, any force transmission cable can be used.

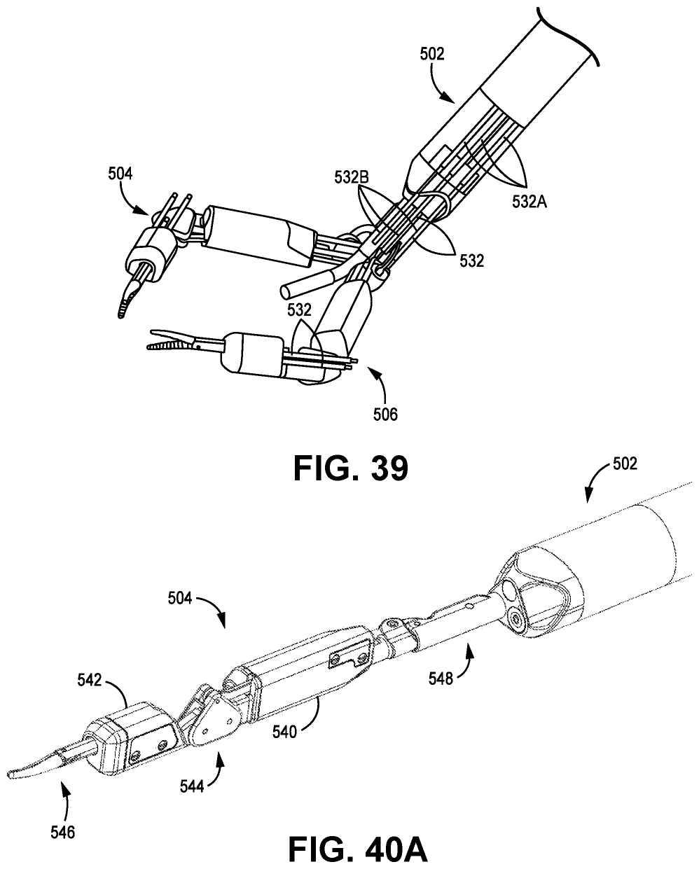

As discussed above, each of the cables 532 extend through the interior of the elongate body 502 and extend out of the distal end of the body 502, as shown in FIG. 39 according to one embodiment. More specifically, some exemplary cables 532 are visible extending through and out of the distal end of the body 502 in the figure. These particular cables 532 are coupled to the intended actuable components in the forearm of the left arm 506. However, the lengths of the cables 532 extending along an exterior of the upper arm of the left arm 506 are not shown. Thus, a length of each of the cables 532 is visible extending distally out of the body 502 as discussed above, and a corresponding length of each of the cables 532 is depicted extending proximally out of the forearm.

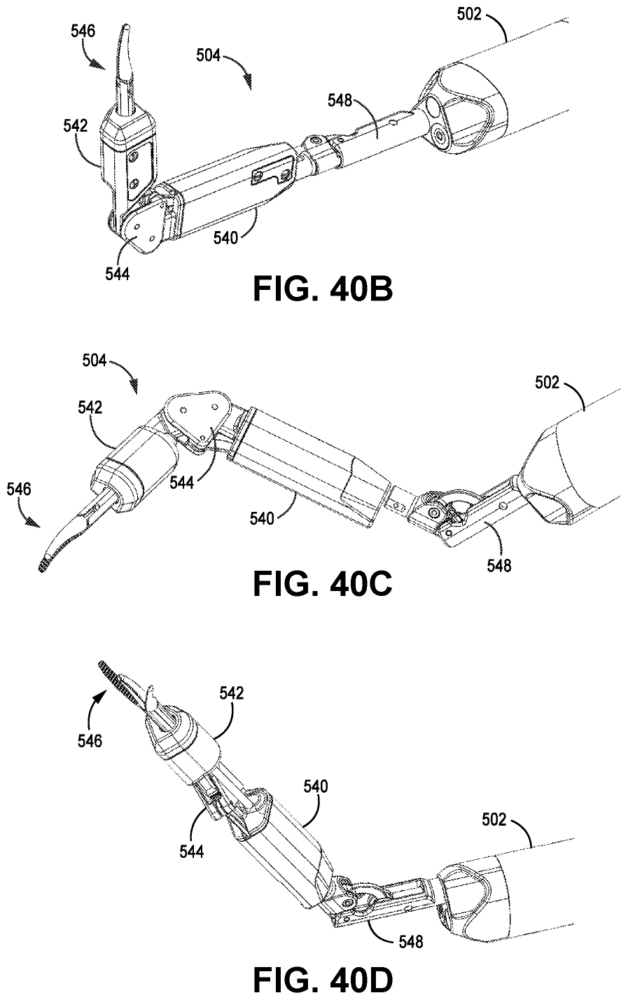

The various actuation assemblies herein (with each actuation assembly made up an actuator, a cable, and an actuable component) will be described in additional detail below. Both arms 504, 506 have six degrees of freedom, which means that each arm 504, 506 has six actuation assemblies operably coupled thereto (with six actuators disposed in the actuation unit 512, as discussed above). As best shown in FIGS. 40A-40D, the right arm 504 in this specific implementation has a first link (or “upper arm”) 540, a second link (or “forearm”) 542 coupled to the upper arm 540 at an elbow joint (or “elbow”) 544, and a removable end effector 546 operably coupled to the forearm 542. The right arm 504 is operably coupled to the elongate body 502 via the shoulder (or “shoulder joint” or “shoulder housing”) 548. While the right arm 504 and the various actuation assemblies related thereto will be discussed in detail here, it is understood that the left arm 506 is substantially similar and has the same components, actuation assemblies, and features therein.

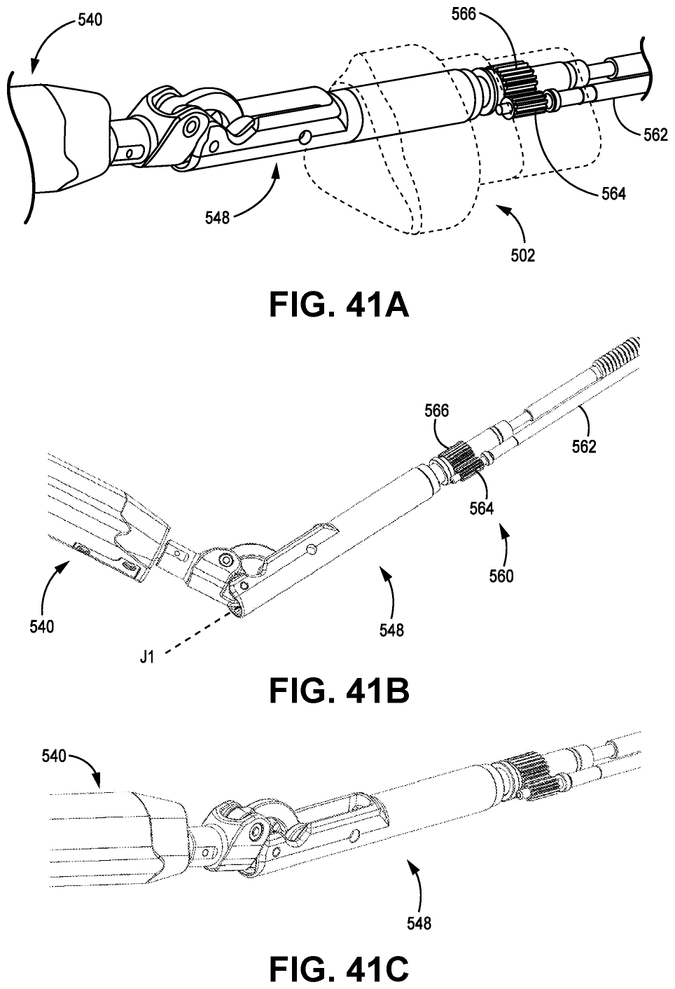

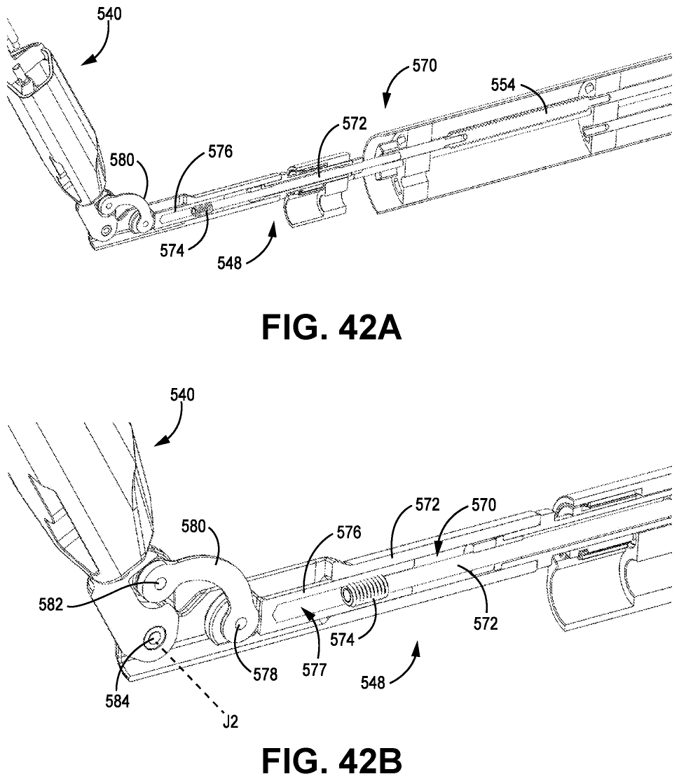

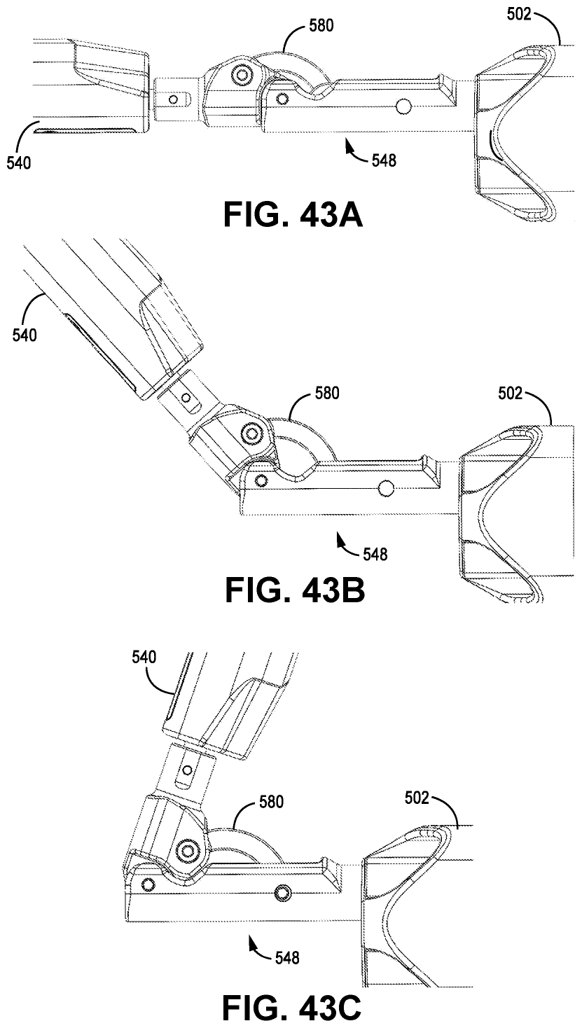

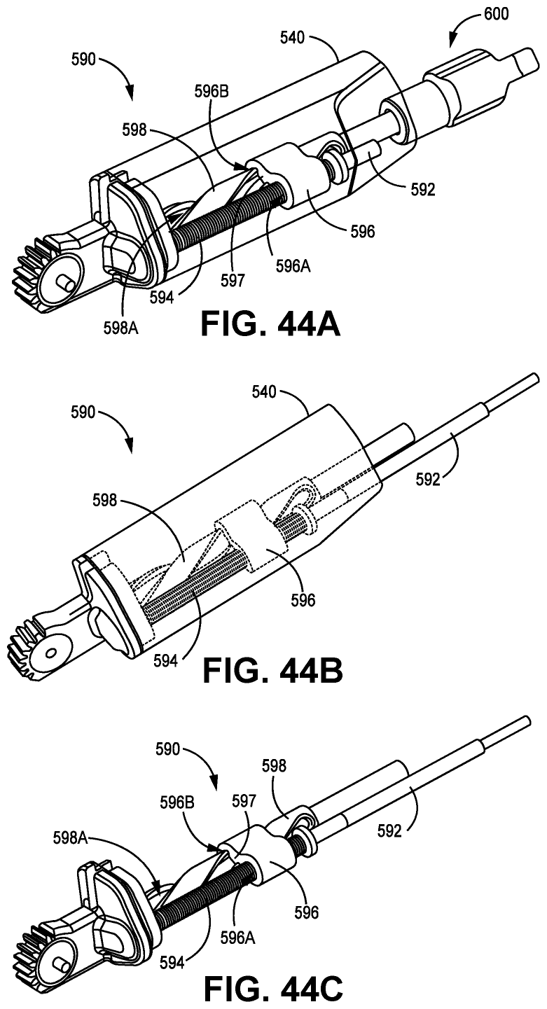

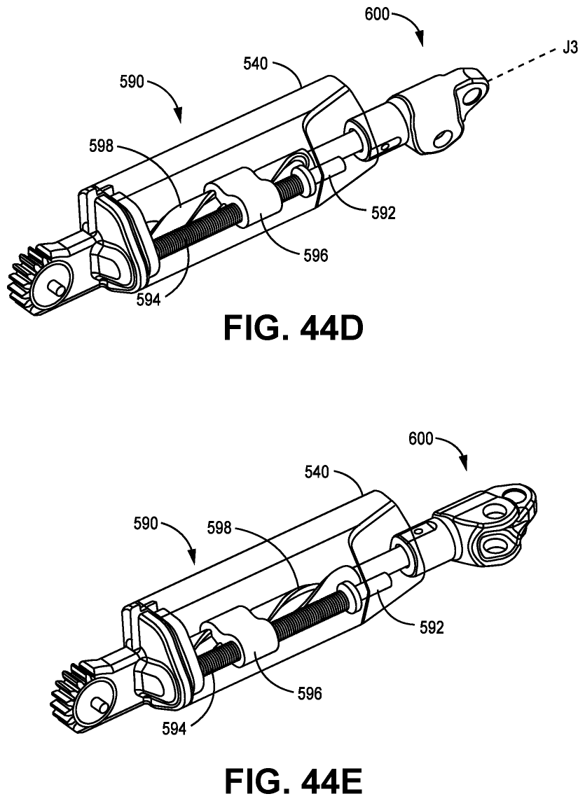

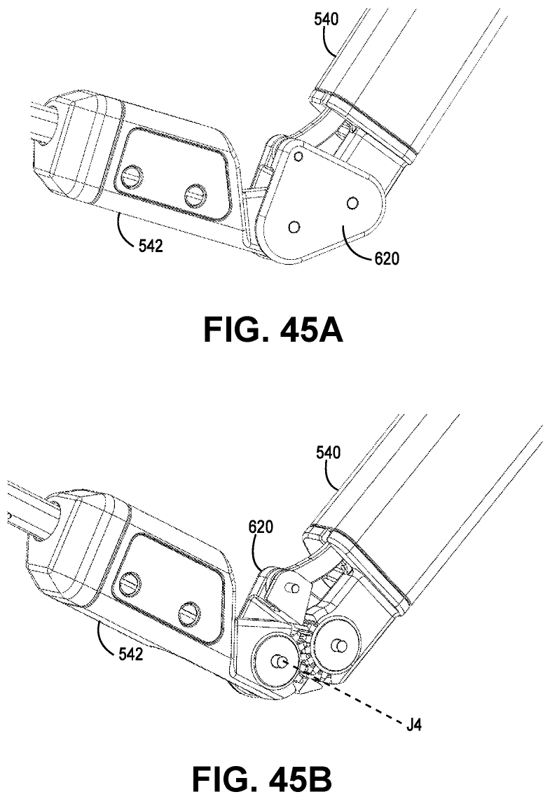

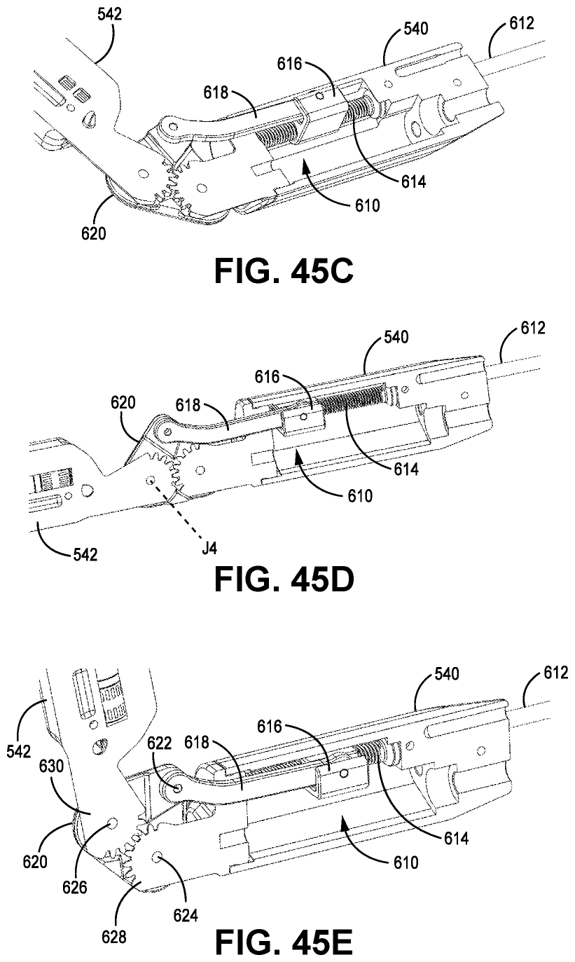

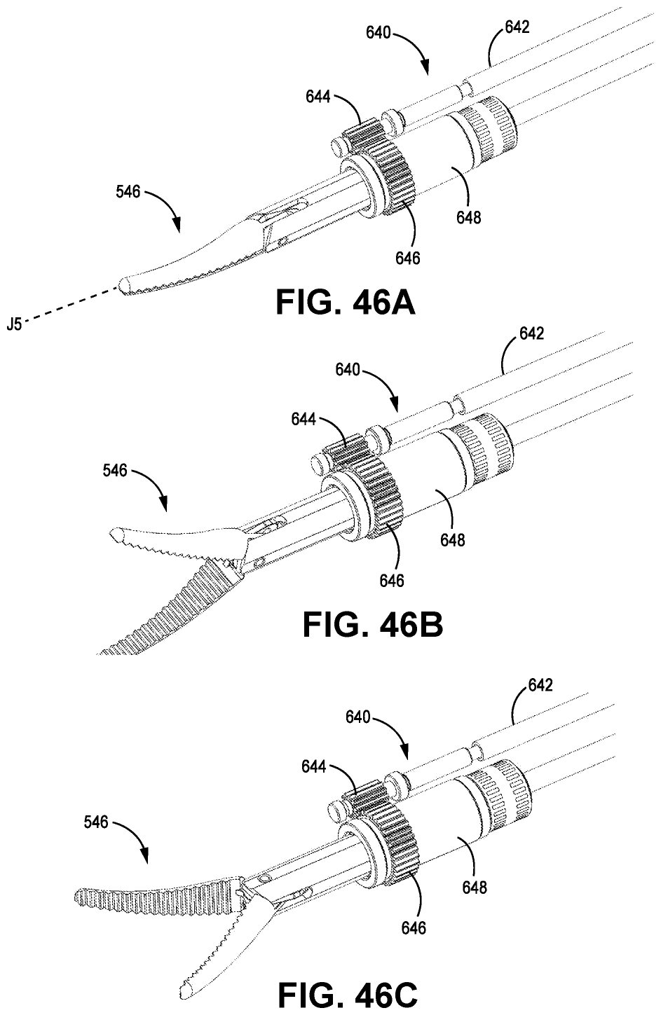

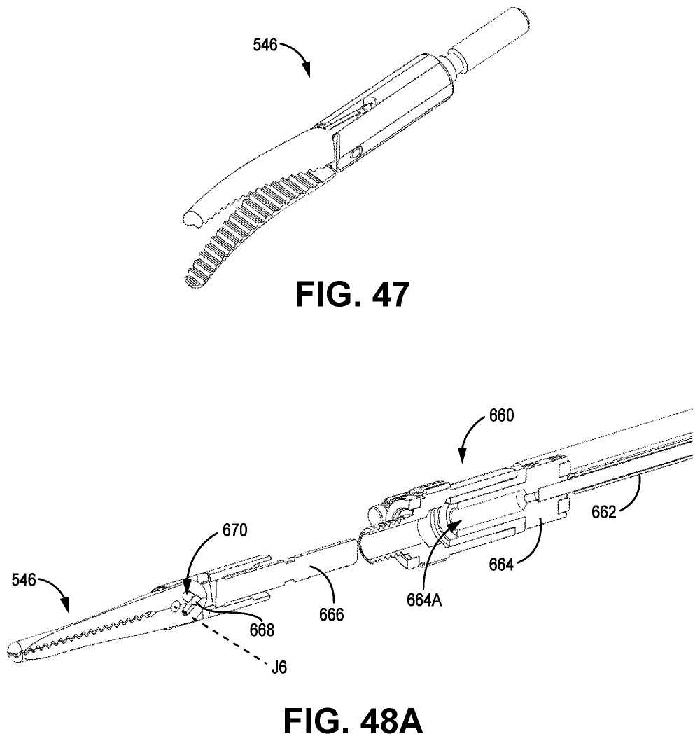

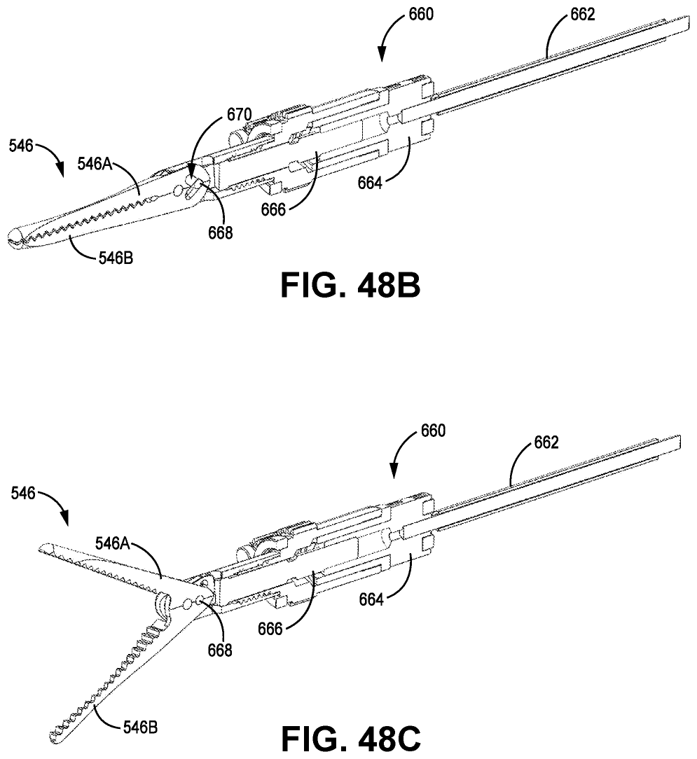

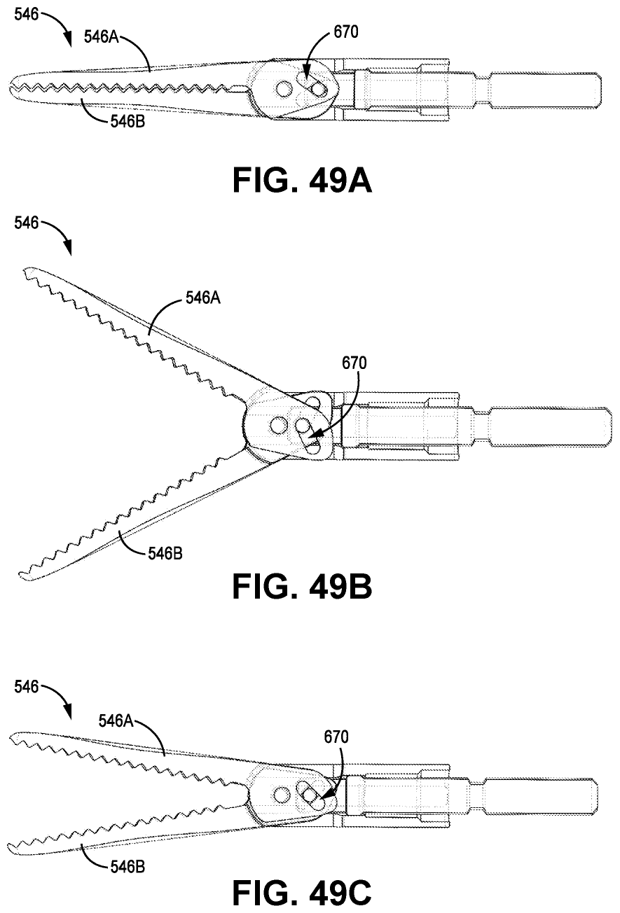

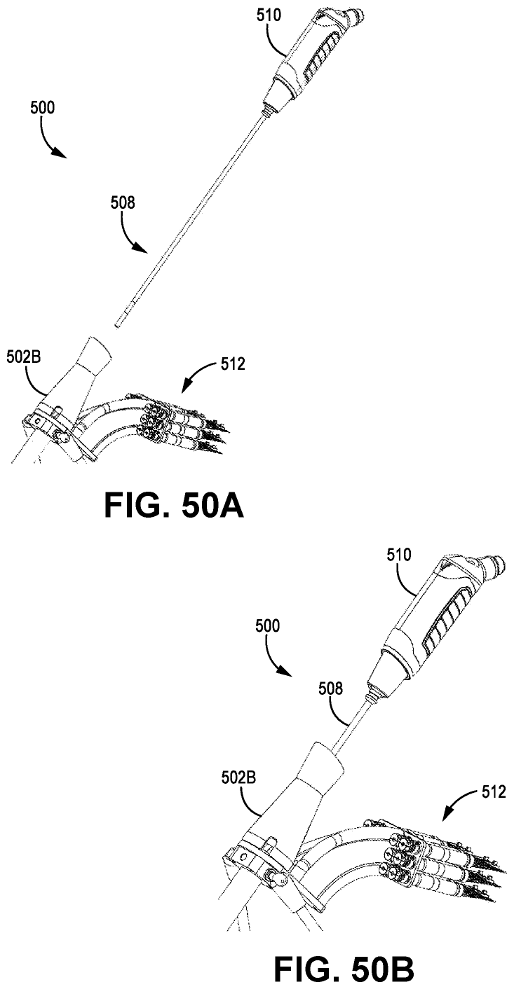

According to one embodiment, the first degree of freedom or axis of rotation of the right arm 504 is the shoulder roll or “yaw.” More specifically, as shown in FIGS. 41A-410 according to one embodiment, the shoulder roll actuation assembly 560 includes an actuator (not shown), a rotary transmission cable 562, a cable gear 564, a driven gear 566, and the actuable component coupled thereto, which in this case is the shoulder housing 548. As discussed above, the cable 562 is coupled at its proximal end to an actuator (not shown) in the actuation unit 512 and extends from the actuation unit 512 through the opening 530 in the elongate body 502 and along the length of the elongate body 502 toward the distal end of the body 502 as best shown in FIG. 41A. The cable gear 564 is fixedly attached (or rotationally constrained) to the distal end of the cable 562 such that rotation of the cable 562 causes rotation of the gear 564. The cable gear 564 is rotatably coupled to the driven gear 566 such that rotation of the cable gear 564 causes rotation of the driven gear 566. Further, the driven gear 566 is fixed attached (or rotationally constrained) to the shoulder housing 548 such that rotation of the driven gear 566 results in rotation of the shoulder housing 548. In certain embodiments (including the exemplary embodiment as shown), both gears 564, 566 have external teeth that mesh together such that the rotation of the cable gear 564 causes rotation of the driven gear 566. Alternatively, any known mechanism(s) can be used to rotatably couple the cable 562 to the shoulder housing 548.

According to one embodiment, the right arm yaw actuation assembly 560 operates in the following fashion. The right arm yaw actuator (now shown) in the actuation unit 512 is actuated to generate motive force, which is transmitted via a physical coupling to the rotary drive cable 562. The rotation of the drive cable 562 causes rotation of the cable gear 564, which causes rotation of the driven gear 566. Further, the rotation of the driven gear 566 causes rotation of the shoulder housing 548 around the rotational axis J1 as best shown in FIG. 41B. This rotation of the shoulder housing 548 results in the shoulder roll or yaw rotation of the right arm 504 as best shown in FIGS. 41B and 41C.

In accordance with a further embodiment, the second degree of freedom or axis of rotation of the right arm 504 is the shoulder pitch. More specifically, as shown in FIGS. 42A-42B according to one embodiment, the shoulder pitch actuation assembly 570 includes an actuator (not shown), a rotary transmission cable 572, cable threaded end 574, a translation rod 576, and a coupling link (or arm) 578 coupled to the actuable component, which in this case is the upper arm 540. As discussed above, the cable 572 is coupled at its proximal end to an actuator (not shown) in the actuation unit 512 and extends from the actuation unit 512 through the opening 530 in the elongate body 502 and along the length of the elongate body 502 toward the distal end of the body 502 as shown in FIGS. 42A-B. The threaded screw (or “end”) 574 is fixedly attached (or rotationally constrained) to the distal end of the cable 572 such that rotation of the cable 572 causes rotation of the screw 574. Alternatively, the threaded screw 574 can be any known rotational component (such as a gear or other such mechanism or component) for coupling to the translation rod 576. As best shown in FIG. 42B, the threaded screw 574 is rotatable disposed within the lumen 577 of the slidable translation rod 576. The inner surface of the lumen 577 is also threaded such that the threaded screw 574 threadably couples with the translation rod 576. The translation rod 576 can be any structure, such as a tube, a block, or any other structure or shape having a threaded lumen that allows for conversion of the rotational motion of the cable 572 to translation or axial motion of the rod 576. Thus, rotation of the threaded screw 574 within the lumen 577 causes the translation rod 576 to move axially within the shoulder housing 548. Further, the translation rod 576 is rotatably attached to a coupling link (or “arm”) 580 at a first rotatable joint 578 at one end of the link 580 such that axial movement of the rod 576 causes movement of the link 580. Further, the coupling link 580 is rotatably coupled to the upper arm 540 of the right arm 504 at a second rotatable joint 582 at the other end of the link 580 such that the movement of the link 580 causes movement of the upper arm 540. Alternatively, any known rotation-to-translation mechanism can be used to moveably couple the cable 572 to the upper arm 540.

According to one embodiment, the right arm shoulder pitch actuation assembly 570 operates in the following fashion. The right arm shoulder pitch actuator (not shown) in the actuation unit 512 is actuated to generate motive force, which is transmitted via a physical coupling to the rotary drive cable 572. The rotation of the drive cable 572 causes rotation of the threaded screw 574, which causes translation (axial movement) of the translation rod 576 as described above. The axial movement of the rod 576 causes movement of the coupling arm 580, which causes rotation of the upper arm 540 around the rotational axis J2 as best shown in FIG. 42B. This rotation of the upper arm 540 results in the shoulder pitch rotation of the right arm 504 as best shown in FIGS. 43A-43C.

The third degree of freedom or axis of rotation of the right arm 504, in certain implementations, is the upper arm roll. More specifically, as shown in FIGS. 44A-44C according to one embodiment, the upper arm roll actuation assembly 590 includes an actuator (not shown), a rotary transmission cable 592, cable threaded end 594, a translation nut 596, and a driven shaft 598 coupled to the actuable component, which in this case is the shoulder coupling component 600. As discussed above, the cable 592 is coupled at its proximal end to an actuator (not shown) in the actuation unit 512 and extends from the actuation unit 512 through the opening 530 in the elongate body 502, along the length of the elongate body 502 and out of the distal end of the body 502 (as shown for example in FIG. 39) and then extends into the upper arm 540 as best shown in FIG. 44B. The threaded screw (or “end”) 594 is fixedly attached (or rotationally constrained) to the distal end of the cable 592 such that rotation of the cable 592 causes rotation of the screw 594. Alternatively, the threaded screw 594 can be any known rotational component (such as a gear or other such mechanism or component) for coupling to the translation nut 596. As shown in FIGS. 44A-44C, according to one embodiment, the translation nut 596 has two lumens defined therethrough: a screw lumen 596A and a driven shaft lumen 596B. The screw lumen 596A receives the cable screw 594 and has a threaded inner surface (not shown) that threadably couples to the threads of the threaded screw 594. The driven shaft lumen 596B receives the driven shaft 598 and has a protrusion 597 therein such that the protrusion 597 matches with and is disposed within the groove 598A of the driven shaft 598 as best shown in FIG. 44C. Thus, the threaded screw 594 is rotatably disposed within the screw lumen 596A and the driven shaft 598 is rotatably disposed within the shaft lumen 596B. The translation nut 596 can be any structure, such as a barrel, a block, or any other structure or shape having two lumens defined therein that allow for conversion of the rotational motion of the cable 592 to translation or axial motion of the nut 596 and then conversion of that translation back to rotational motion of the drive shaft 598. Thus, rotation of the threaded screw 594 within the lumen 596A causes the translation nut 596 to move axially within the upper arm 540. Further, the axial movement of the nut 596 causes the driven shaft 598 to rotate due to the protrusion 597 being disposed within the groove 598A that winds around the shaft 598. The driven shaft 598 is fixedly attached (or rotationally constrained) to the shoulder coupling component 600 such that rotation of the shaft 598 causes rotation of the coupling component 600. Alternatively, any known rotation-to-translation-to-rotation (or just rotation-to-rotation) mechanism can be used to moveably couple the cable 592 to the shoulder coupling component 600.

According to one embodiment, the right upper arm roll actuation assembly 590 operates in the following fashion. The right upper arm roll actuator (not shown) in the actuation unit 512 is actuated to generate motive force, which is transmitted via a physical coupling to the rotary drive cable 592. The rotation of the drive cable 592 causes rotation of the threaded screw 594, which causes translation (axial movement) of the translation nut 596 as described above. This translation causes rotation of the driven shaft 598 as also described above. The rotation of the driven shaft 598 causes rotation of the shoulder coupling component 600, which causes rotation of the upper arm 540 around the rotational axis J3 as best shown in FIG. 44D. This rotation of the upper arm 540 results in the roll of the right upper arm 540 around that axis as a result of the rotation of the coupling component 600 as best shown in FIGS. 44D and 44E.

In another aspect, the fourth degree of freedom or axis of rotation of the right arm 504 is the elbow pivot. More specifically, as shown in FIGS. 45A-45E according to one embodiment, the elbow pivot actuation assembly 610 includes an actuator (not shown), a rotary transmission cable 612, cable threaded end 614, a translation block 616, and a coupling link (or arm) 618 coupled to the actuable component, which in this case is the elbow housing 620. As discussed above, the cable 612 is coupled at its proximal end to an actuator (not shown) in the actuation unit 512 and extends from the actuation unit 512 through the opening 530 in the elongate body 502, along the length of the elongate body 502, out of the distal end of the body 502 (as shown for example in FIG. 39) and then extends into the upper arm 540 as best shown in FIGS. 45C-E. The threaded screw (or “end”) 614 is fixedly attached (or rotationally constrained) to the distal end of the cable 612 such that rotation of the cable 612 causes rotation of the screw 614. Alternatively, the threaded screw 614 can be any known rotational component (such as a gear or other such mechanism or component) for coupling to the translation block 616. As best shown in FIGS. 44C and 44E, the translation block 616 has both a screw lumen 616A to receive (and couple to) the screw 614 and an arm slot 616B to receive (and couple to) the coupling arm 618. The screw lumen 616A receives the cable screw 614 and has a threaded inner surface (not shown) that threadably couples to the threads of the threaded screw 614. The arm slot 616B receives one end of the coupling arm 618 and has a rod 617 extending through the slot 616B such that the rod 617 is rotatably coupled with the arm 618 such that the arm can rotate in relation to the translation block 616 around the rod 617. Thus, the threaded screw 614 is rotatably disposed within the screw lumen 616A and the end of the coupling arm 618 is rotatably disposed within the slot 616B as shown. Alternatively, the translation block 616 can be any structure, such as a barrel, a nut, or any other structure or shape having two coupling features defined therein that allow for conversion of the rotational motion of the cable 612 to translation or axial motion of the block 616 and the arm 618 (with the arm being pivotable in relation to the block 616 as disclosed herein).

Further, as best shown in FIG. 45E according to one embodiment, the coupling arm 618 is rotatably attached to the elbow housing 620 at a first rotatable joint 622 at the end of the arm 618 opposite the coupling to the rod 617. Further, the elbow housing 620 is rotatably coupled to a distal extension 628 of the upper arm 540 at a second rotatable joint 624 such that the elbow housing 620 is rotatable in relation to the upper arm 540. As such, axial movement of the arm 618 causes the elbow housing 620 to rotate around the second rotatable joint 624. In addition, the forearm 542 has a proximal extension 630 that is rotatably coupled to the elbow housing 620 at a third rotatable joint 626 such that the forearm 542 is rotatable in relation to the elbow housing 620. According to the exemplary implementation as shown, the distal extension 628 of the upper arm 540 is rotatably coupled to the proximal extension 630 of the forearm 542 such that the outer edge of the proximal extension 630 rotates around and in contact with (and in relation) to the outer edge of the distal extension 628. In certain implementations, both extensions 628, 630 have teeth that mate with each other as shown such that the teeth will cause the forearm 542 to rotate around the third rotatable joint 626 as the proximal extension 630 rotates around the distal extension 628. As such, axial movement of the arm 618 causes the elbow housing 620 to rotate around the second joint 624, which causes the forearm 542 to rotate around the third joint 626. Alternatively, any known rotation-to-translation-to-rotation mechanism can be used to moveably couple the cable 612 to the elbow housing 620 and the forearm 542.

According to one embodiment, the right arm elbow pivot actuation assembly 610 operates in the following fashion. The right arm elbow pivot actuator (not shown) in the actuation unit 512 is actuated to generate motive force, which is transmitted via a physical coupling to the rotary drive cable 612. The rotation of the drive cable 612 causes rotation of the threaded screw 614, which causes translation (axial movement) of the translation block 616 as described above. The axial movement of the block 616 causes axial movement of the coupling arm 618, which causes rotation of the elbow housing 620 around the rotatable joint 624, which cause rotation of forearm 542 around the rotatable joint 626, which is the rotational axis J4 as best shown in FIGS. 45B and 45D. This rotation of the forearm 542 results in the elbow pivot rotation of the right arm 504 as best shown in FIGS. 45C-45E.

According to some implementations, the fifth degree of freedom or axis of rotation of the right arm 504 is the end effector roll. More specifically, as shown in FIGS. 46A-46C according to one embodiment, the end effector roll actuation assembly 640 includes an actuator (not shown), a rotary transmission cable 642, a cable gear 644, a driven gear 646, and the actuable component coupled thereto, which in this case is the end effector housing 648. As discussed above, the cable 642 is coupled at its proximal end to an actuator (not shown) in the actuation unit 512 and extends from the actuation unit 512 through the opening 530 in the elongate body 502, along the length of the elongate body 502, out of the distal end of the body 502 (as shown for example in FIG. 39) and then extends into the forearm 542 as best shown in FIGS. 46A-46C (wherein the forearm 542 itself is not shown). The cable gear 644 is fixedly attached (or rotationally constrained) to the distal end of the cable 642 such that rotation of the cable 642 causes rotation of the gear 644. The cable gear 644 is rotatably coupled to the driven gear 646 such that rotation of the cable gear 644 causes rotation of the driven gear 646. Further, the driven gear 646 is fixed attached (or rotationally constrained) to the end effector housing 648 such that rotation of the driven gear 646 results in rotation of the end effector housing 648. In certain embodiments (including the exemplary embodiment as shown), both gears 644, 646 have external teeth that mesh together such that the rotation of the cable gear 644 causes rotation of the driven gear 646. Alternatively, any known mechanism(s) can be used to rotatably couple the cable 642 to the end effector housing 648.

According to one embodiment, the right arm end effector roll actuation assembly 640 operates in the following fashion. The right arm end effector roll actuator (not shown) in the actuation unit 512 is actuated to generate motive force, which is transmitted via a physical coupling to the rotary drive cable 642. The rotation of the drive cable 642 causes rotation of the cable gear 644, which causes rotation of the driven gear 646. Further, the rotation of the driven gear 646 causes rotation of the end effector housing 648 (and thus the end effector 546) around the rotational axis J5 as best shown in FIG. 46A. This rotation of the end effector housing 648 results in the end effector roll of the right arm 504 as best shown in FIGS. 46B and 46C.

In accordance with a further embodiment, the sixth degree of freedom or axis of rotation of the right arm 504 is the end effector open/close actuation. More specifically, as shown in FIGS. 47-49C according to one embodiment, the end effector actuation assembly 660 includes an actuator (not shown), a rotary transmission cable 662, cable female drive barrel 664, a translation rod 666, and a protrusion 668 coupled to the actuable component, which in this case is the end effector 546. As discussed above, the cable 662 is coupled at its proximal end to an actuator (not shown) in the actuation unit 512 and extends from the actuation unit 512 through the opening 530 in the elongate body 502, along the length of the elongate body 502, out of the distal end of the body 502 (as shown for example in FIG. 39), and then extends into and through the forearm 542 as best shown in FIGS. 48A-48C (wherein the forearm 542 itself is not shown). The female drive barrel 664 is fixedly attached (or rotationally constrained) to the distal end of the cable 662 such that rotation of the cable 662 causes rotation of the barrel 664. Alternatively, the drive barrel 664 can be any known rotational component with a female opening (such as a tube or other such mechanism or component) for coupling to the translation rod 666, or any other rotation-to-translation component or mechanism. As best shown in FIG. 48A, the drive barrel 664 has a lumen 664A defined therein with an opening at the distal end thereof to receive the translation rod 666. In this exemplary implementation, the lumen 664A has threads on the inner surface of the lumen 664A. The translation rod 666 is rotatably disposed within the lumen 664A of the barrel 664. Further, the outer surface of the rod 666 is threaded such that the threads of the rod 666 mate with the threads of the inner surface of the lumen 664A. As such, rotation of the drive barrel 664 causes translation or axial motion of the rod 666. Further, the translation rod 666 has a radial protrusion 668 at the distal end of the rod 666 (or alternatively, the rod 666 is fixedly attached to a radial protrusion 668). In addition, the two grasper arms 546A, 546B of the grasper end effector 546 have slots 670 defined within the proximal ends of the arms 546A, 546B that are sized and configured to receive the protrusion 668 therein such that axial movement of the protrusion 668 causes the grasper arms 546A, 546B to move between their open and closed positions. Alternatively, any known rotation-to-translation-to-transverse-rotation (or rotation-to-transverse-rotation) mechanism can be used to moveably couple the cable 662 to the end effector 546.

According to one embodiment, the right arm end effector open/close actuation assembly 660 operates in the following fashion. The right arm shoulder pitch actuator (not shown) in the actuation unit 512 is actuated to generate motive force, which is transmitted via a physical coupling to the rotary drive cable 662. The rotation of the drive cable 662 causes rotation of the drive barrel 664, which causes translation (axial movement) of the translation rod 666 as described above. The axial movement of the rod 666 causes axial movement of the protrusion 668, which causes rotation of the grasper arms 546A, 546B around the rotational axis J6 as best shown in FIG. 48A. More specifically, the axial movement of the rod 666 (and protrusion 668) causes rotation of the grasper arms 546A, 546B between a closed configuration as shown in FIG. 49A, an open configuration as shown in FIG. 49B, and any position of the two arms 546A, 546B therebetween, such as the position as shown in FIG. 49C.

FIGS. 50A and 50B depict one implementation of the device 500 with the removable camera 508. More specifically, in contrast to FIGS. 35A-36B in which the removable camera 508 is disposed within and fully attached to the elongate body 502, FIG. 50A depicts the camera 508 in position to be inserted into the device 500. Further, FIG. 50B depicts the camera 508 being urged into the fully attached or docked position such that the elongate tube 508A of the camera 508 is extending out of the proximal section 502B of the elongate body 502. In one embodiment, the camera 508 and the interface with the device 500 can be substantially similar to the device 40 embodiment or any other embodiments described above, or alternatively can be substantially similar to any of the camera and interface components and/or features of the devices disclosed in U.S. application Ser. Nos. 16/736,329, 16/926,025, 17/075,122, and 17/367,915, all of which are incorporated herein by reference in their entireties.

Returning to FIGS. 38A-38D, certain embodiments of the device 500 can also include a mechanism to control and/or manage the movement of the drive cables 532 in relation to the elongate device body 502 to avoid excessive slack in the cables 532. More specifically, as discussed in further detail above and depicted in FIGS. 38A-B and 39, several of the cables 532 extend from the distal end of the elongate body 502 to the forearm 542 such that the lengths of those cables 532 between the elongate body 502 and the forearm 542 are disposed outside of the body 502 and arm 504. Thus, when the arms 504, 506 extend into their fully extended positions (as shown with respect to arm 504 in FIG. 40A, for example), it is necessary for the cables 532 have sufficient length to extend the full distance between the distal end of the elongate body 502 and the forearm 542. In contrast, when the arms 504, 506 are disposed such that the forearms 542 are closer to the distal end of the elongate body 502, the external cables 532 have excess length. As such, these externally disposed cables 532 could cause problems if they have too much slack in them (including possible snagging on certain movable components of the device 500 during use) based on the position of the arms 504.

In this exemplary embodiment, the cable positioning block (or “insert”) 555 controls the amount of the cable length of the cables 532 extending out of the distal end of the elongate body 502 depending on the position and movement of the arms 504, 506, thereby eliminating the excess cable length that could create problems. As best shown in FIGS. 38C, 38D, and 42A, the cable block actuation assembly is coupled to the shoulder pitch actuation assembly 570 such that both assemblies utilize the same rotary transmission cable 572. The cable 572 has a cable gear 554 disposed at a midpoint along the length of the cable 572 as best shown in FIG. 42A such that actuation of the shoulder pitch actuation assembly 570 also causes actuation of the cable gear 554 and the actuable component coupled thereto, which in this case is the positioning block 555. That is, the cable gear 554 is fixedly attached (or rotationally constrained) or incorporated into a midpoint of the length of the cable 553 such that rotation of the cable 553 causes rotation of the gear 554. The cable gear 554 is rotatably disposed within a first lumen 556 of the cable block 555, wherein the inner surface of the lumen 556 is threaded (as is the cable gear 554) such that rotation of the cable gear 564 causes translation or axial movement of the cable block 555. Further, an elongate support 557 is disposed within and extends along the length of the elongate body 502 and is disposed through a second lumen 558 in the block 555 such that the block 555 is slidable along the support 557 via the second lumen 558. As such, the support 557 helps to ensure that the block 555 maintains its radial disposition as it is urged axially. Alternatively, any known rotation-to-translation mechanism(s) can be used to rotatably couple the cable 553 to the block 555 and thereby actuate the block 555 as described herein.

According to one embodiment, the cable positioning block actuation assembly operates in the following fashion. The shoulder pitch actuator (not shown) in the actuation unit 512 (or elsewhere) is actuated to generate motive force and cause the upper arm 540 to rotate in relation to the elongate body 502 into any of the positions as shown in FIGS. 43A-43C. As discussed above, it is this movement of the upper arm 540 that has the greatest effect on the external cables. Thus, when the shoulder pitch actuator is actuated, it also causes the cable gear 554 to rotate, which causes axial movement of the cable positioning block 555. More specifically, when the upper arm 540 is urged into an acute angle as best shown in FIG. 43C, the cable positioning block 555 is urged into its proximal position as best shown in FIGS. 38B and 38D, which urges the cables 532 into their retracted position at the distal end of the elongate body 502 as best shown in FIG. 38B. Similarly, when the upper arm 540 is urged into its extended or straight position as shown in FIG. 43A, the cable positioning block 555 is urged into its distal position as best shown in FIGS. 38A and 38C, which urges the cables 532 into their extended position at the distal end of the elongate body 502 as best shown in FIG. 38A. As such, the cable positioning block 555 operates to control the length of the external cables.

Continuing with FIGS. 38A-38D, along with FIG. 39, certain embodiments of the device 500 can also include certain improved cable features or characteristics to control and/or manage the radial and rotational flexibility of the drive cables 532. As described elsewhere herein, one of the benefits of the cables 532 is that they are radially flexible (or bendable) such that some can extend distally from the elongate body 502 to the forearm 542 and can flex as needed when the arm 504 moves into different configurations (as best shown in FIG. 39). Further, proximal lengths of the cables 532 (as best shown in FIGS. 36C-36D) must also be radially flexible such that the cables 532 can bend as they extend through the opening 530 and are coupled to the actuators 534, 536 of the actuation unit 512. However, the more radially flexible each cable is, the greater the rotational flexibility of that cable, such that there is risk of the cable bending when actuation of the cable is attempted. Thus, as best shown in FIG. 39, in certain embodiments, each of the cables 532 can have a rigid length 532A disposed within the elongate body 502 such that the cables 532 are not radially flexible or are prevented from any radial movement. In contrast, the cables 532 can have a flexible length 532B along the length that is disposed between the elongate body 502 and the forearm 542. Further, in certain implementations, the cables 532 can have a thick flexible length 532C that is thicker than the flexible length 532B but still flexible along the length that is disposed between the opening 530 and the actuation unit 512 as best shown in FIGS. 36B-36D. Thus, the rigid length 532A of each cable 532 helps to reduce the risk of bending of the flexible length 532B as a result of actuation of the cable 532. Similarly, the thickness of the thick flexible length 532C allows for radial bending of the cables 532 along the length 532C while also reducing the risk of bending of the thick flexible length 532C as a result of actuation of the cable 532.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A robotic device comprising:
(a) an elongate body;
(b) a robotic arm operably coupled to the elongate body, the robotic arm comprising:
(i) an upper arm segment;
(ii) a forearm segment operably coupled to the upper arm segment; and
(iii) at least two actuable components associated with the robotic arm;
(c) a first cable-driven actuation assembly comprising:
(i) a first actuator disposed within one of the elongate body, the upper arm segment, and the forearm segment or external to the device; and
(ii) a motive force transfer cable operably coupled to the first actuator and a first of the at least two actuable components; and
(d) a first gear-driven actuation assembly comprising:
(i) a second actuator disposed within one of the elongate body, the upper arm segment, and the forearm segment or external to the device; and
(ii) at least one gear operably coupled to the second actuator and a second of the at least two actuable components.

2. The device of claim 1, wherein the motive force transfer cable is a rotary force transfer cable.

3. The device of claim 1, wherein the motive force transfer cable is a lateral force transfer cable.

4. The device of claim 3, wherein the lateral force transfer cable comprises a single lateral push/pull cable or two lateral pull cables.

5. The device of claim 1, wherein the first of the at least two actuable components comprises actuable end effector grasper arms, and wherein the second of the at least two actuable components comprises a rotatable end effector grasper body.

6. The device of claim 1, wherein the first and second actuators are disposed within the forearm segment.

7. The device of claim 1, wherein the first actuator is disposed within the upper arm segment and the second actuator is disposed within the forearm segment.

8. The device of claim 1, wherein the first actuator is disposed within the elongate body and the second actuator is disposed within the forearm segment.

9. The device of claim 1, wherein the first actuator is disposed external to the device and the second actuator is disposed within the forearm segment.

10. A robotic device comprising:
(a) an elongate body;
(b) a robotic arm operably coupled to the elongate body, the robotic arm comprising:
(i) an upper arm segment;
(ii) a forearm segment operably coupled to the upper arm segment; and
(iii) at least two actuable components associated with the robotic arm;
(c) a first cable-driven actuation assembly comprising:
(i) a first actuator disposed within one of the elongate body, the upper arm segment, and the forearm segment or external to the device; and
(ii) a first motive force transfer cable operably coupled to the first actuator and a first of the at least two actuable components; and
(d) a second cable-driven actuation assembly comprising:
(i) a second actuator disposed within one of the elongate body, the upper arm segment, and the forearm segment or external to the device; and
(ii) a second motive force transfer cable operably coupled to the second actuator and a second of the at least two actuable components.

11. The robotic device of claim 10, further comprising a first gear-driven actuation assembly comprising:
(a) a third actuator disposed within one of the elongate body, the upper arm segment, and the forearm segment or external to the device; and
(b) at least one gear operably coupled to the third actuator and a third of the at least two actuable components.

12. The device of claim 10, wherein at least one of the first and second motive force transfer cables is a rotary force transfer cable.

13. The device of claim 10, wherein at least one of the first and second motive force transfer cables is a lateral force transfer cable.

14. The device of claim 10, wherein the first of the at least two actuable components comprises actuable end effector grasper arms, and wherein the second of the at least two actuable components comprises a rotatable end effector grasper body.

15. The device of claim 10, wherein the first and second actuators are disposed within the forearm segment.

16. The device of claim 10, wherein the first and second actuators are disposed external to the device.

17. A robotic device comprising:
(a) an elongate body;
(b) an actuation unit coupled to the elongate body, the actuation unit comprising at least two actuators;
(c) a robotic arm operably coupled to the elongate body, the robotic arm comprising:
(i) an upper arm segment;
(ii) a forearm segment operably coupled to the upper arm segment; and
(iii) at least two actuable components associated with the robotic arm;
(d) a first cable-driven actuation assembly comprising:
(i) a first actuator disposed within the actuation unit; and
(ii) a first rotary force transfer cable operably coupled to the first actuator and a first of the at least two actuable components; and
(e) a second cable-driven actuation assembly comprising:
(i) a second actuator disposed within the actuation unit; and
(ii) a second rotary force transfer cable operably coupled to the second actuator and a second of the at least two actuable components.

18. The device of claim 17, wherein the first and second rotary force transfer cables are disposed through the elongate body.

19. The device of claim 18, further comprising a cable positioning block movably disposed within the elongate body, wherein the cable positioning block is operably coupled to the first rotary force transfer cable, and wherein the second rotary force transfer cable is attached to the cable positioning block.

20. The device of claim 17, wherein the first rotary force transfer cable is disposed through an opening in the cable positioning block such that the first rotary force transfer cable is rotatably coupled to the cable positioning block such that rotation of the first rotary force transfer cable results in axial movement of the cable positioning block with in the elongate body.
